(19) United States
(12) Patent Application Publication
(10) Pub. No.: US 2003/0124091 A1
(43) Pub. Date: Jul. 3, 2003

(54) Endothelial cell derived hematopoietic growth factor

(75) Inventors: Daniel Tuse, Vacaville, CA (US); Thomas A. Davis, Oak Hill, VA (US); Alison A. McCormick, Vacaville, CA (US); Sharon L. Wannberg, Germantown, MD (US)

Correspondence Address: SUGHRUE MION, PLLC, 1010 El Camino Real, Menlo Park, CA, 94025-4345, US

(73) Assignee: Large Scale Biology Corporation

(21) Appl. No.: 10/281,423

(22) Filed: Oct. 25, 2002

Related U.S. Application Data
(60) Provisional application No. 60/348,903, filed on Oct. 26, 2001.
(60) Provisional application No. 60/344,680, filed on Oct. 31, 2001.
(60) Provisional application No. 60/338,309, filed on Dec. 6, 2001.
(60) Provisional application No. 60/364,799, filed on Mar. 15, 2002.
(60) Provisional application No. 60/372,498, filed on Apr. 11, 2002.

(51) Int. Cl. A61K 38/20; A61K 38/19; A61K 38/18; A61K 39/00

(52) U.S. Cl. 424/85.1; 424/85.2; 424/185.1; 514/12

(57) ABSTRACT

The present invention relates to human and porcine endothelial cell derived growth factors (EDHF) that contain one or a mixture of more than one endothelial cell proteins having a molecular weight greater than about 30 kDa. The EDHF is added to culture medium to expand tri-lineage pre-dendritic myleomonocytic progenitor cells and culture endothelial cells. The present invention also relates to a method of amplifying myeloid dendritic cell precursors both in vitro and in vivo. The EDHF is also used therapeutically to increase myeloid dendritic cell production in vivo to enhance the activity of vaccines.

This application claims benefit of Provisional Application No. 60/348,903 filed Oct. 26, 2001, Provisional Application No. 60/344,680 filed Oct. 31, 2001, Provisional Application No. 60/338,309 filed Dec. 6, 2001, Provisional Application No. 60/364,799 filed Mar. 15, 2002, and Provisional Application No. 60/372,498 filed Apr. 11, 2002; the disclosures of which are incorporated herein by reference.

### BACKGROUND OF THE INVENTION

1. Field of the Invention

The present invention relates to porcine endothelial cell derived hematopoietic growth factor (EDHF) that is used in vitro to generate tri-lineage pre-dendritic myleomonocytic progenitor cells from hematopoietic stem and progenitor cells. In vivo EDHF serves as a therapeutic agent to stimulate hematopoiesis and enhance the effectiveness of vaccines. In particular, the present invention relates to the generation and robust amplification/expansion of tri-lineage pre-dendritic myleomonocytic progenitor cells from hematopoietic stem and progenitor cells by culturing these cells with EDHF, one or more human or porcine endothelial cell derived hematopoietic growth factor proteins having a molecular weight (MW) greater than about 30 kDa. The endothelial cell proteins having a molecular weight (MW) greater than about 30 kDa can also be administered to a mammal to stimulate hematopoiesis, hematopoietic progenitor cell expansion, stem cell mobilization, and immune responses.

2. Description of the Prior Art

Hematopoiesis is the process by which blood cells develop and differentiate from pluripotent stem cells in the bone marrow. The pluripotent stem cell is able to renew itself as well as to give rise to committed progenitor cells such as the erythroid, myeloid, and lymphoid progenitors. The progenitor cells, in turn, give rise to differentiated cells which are morphologically recognizable as belonging to a certain lineage such as the erythroid, megakaryocytic, myeloid, lymphoid, and dendritic cell (DC) lineages, and which have a limited or no capacity to proliferate. In humans, stem cells and progenitor cells express the CD34 antigen; while more differentiated hematopoietic precursor cells do not. This process involves a complex interplay of polypeptide growth factors (cytokines) acting via membrane-bound receptors on the target cells. Cytokine action results in cellular proliferation and differentiation, with response to a particular cytokine often being lineage-specific and/or stage-specific. Development of a single cell type, such as a neutrophil or dendritic cell, from a hematopoietic stem cell may require the coordinated action of a plurality of cytokines acting in the proper sequence.

The known cytokines include the interleukins, such as IL-1, IL-2, IL-3, IL-6, IL-8, etc.; and the colony stimulating factors, such as G-CSF, M-CSF, GM-CSF, erythropoietin (EPO), stem cell factor (SCF), flt3 ligand (FLT3L), etc. In general, the interleukins act as mediators of immune and inflammatory responses. The colony stimulating factors stimulate the proliferation of marrow-derived cells, activate mature leukocytes, and otherwise form an integral part of the host's response to inflammatory, infectious, and immunologic challenges.

Hematopoietic stem and progenitor cells, isolated from bone marrow, peripheral blood, cord blood, or fetal liver, when stimulated by SCF or FLT3L alone show little growth response, but both cytokines in combination with other early and late acting cytokines (such as IL-1, IL-3, G-CSF, GM-CSF, and TPO) synergistically enhance the growth in a direct manner. SCF and FLT3L have been shown to be useful for peripheral stem cell mobilization applications, when co-administered with a second cytokine such as GM-CSF or G-CSF, and in expanding bone marrow stem and progenitor cells numbers in vivo. Although both SCF and FLT3L stimulate the production of DC from CD34⁺ hematopoietic progenitor cells in vitro, to date only FLT3L has reported to stimulate DC generation in vivo.

Immunization requires the coupled introduction of antigen with adjuvant to attain an optimal inflammatory reaction. Among the professional antigen presenting cells (APC), dendritic cells (DC) are thought to play the pivotal role in antigen presentation to, and activation of, naive T-cells and B-cells. When loaded with antigens that are accessible to class I or class II major histocompatibility complex (MHC) molecules, dendritic cells can prime resting or naive T cells and generate memory T-cell responses in vitro and in vivo without additional exogenous adjuvant. Antigen uptake, processing and presentation by professional APC are requisite steps in the activation of naive CD4⁺ T cells and initiation of the primary immune response. In vitro data emphasize that DC drive strong CD4⁺, predominantly TH1 responses. DC are found in all lymphoid and non-lymphoid tissues, and are also referred to as Langerhan cells (skin), interdigitating cells (lymphoid tissues) and veil cells (lymph and blood). In humans, dendritic cells arise from the CD34 multipotential hematopoietic progenitor cell population that contains the pluripotent stem cell subset. DC can be generated in vitro from bone marrow, cord blood and mobilized CD34 progenitor cells using combinations of cytokines [granulocyte-macrophage colony-stimulating factor (GM-CSF), tumor necrosis factor alpha (TNF-α)], and interleukin-4 (IL-4). Phenotypically, DCs lack myeloid lineage-specific markers, and express high levels of CD1a and MHC class II, costimulatory molecules CD80 and CD86, and the dendritic cell surface marker CD83.

Caux et al described the culture of CD34⁺ cord blood cells in GM-CSF and TNF-α to produce a population of CD1a⁺ cells (5% to 15% of the progeny) with marked allostimulatory activity. Yields were approximately 10⁷ CD1a cells from 10⁶ CD34⁺ cells at 2 weeks. Young et al have suggested that SCF, GM-CSF, and TNF-α provided optimal conditions to allow at least a significant percentage of DC to grow in liquid culture. Assessment of the cells produced suggests approximately 10% to 15% are differentiated human DC. Overall yields are of the order of 10⁶ DC cells from 10⁴ CD34⁺ BM cells after 3 to 4 weeks. Mobilized CD34⁺ cells have been used as another source of progenitors for attempts to grow DC. Bernhard et al. used GM-CSF to culture CD34⁺ progenitors producing 30% to 60% CD1a⁺ cells within a 20- to 40-fold expanded total cell population at day 15. Mackensen et al added GM-CSF and IL-4 to a cocktail of SCF, EPO, IL-ILβ, IL-3, and IL-6, thereby generating a high proportion (45%) of CD1a⁺ cells. Siena et al used SCF and flt-3 ligand to supplement GM-CSF and TNF-a culture of CD34⁺ mobilized cells. Yields were of a similar order (4×10⁷ cells from 10⁶ CD34⁺ cells) with 33% to 55% CD1a⁺ cells in the progeny.

The addition of either SCF and/or FLT3L to the stimulation mixture of cytokines increases the production of DC from CD34⁺ progenitor cells in combination with GM-CSF plus TNF-α plus IL-4. As with SCF, FLT3L does not appear to affect the differentiation, but rather the production, of DC. Production of DC from mobilized CD34⁺ peripheral blood progenitor cells (PBPC) by GM-CSF and TNF-α is enhanced by SCF and FLT3L individually; combining them results in an additive response. It has been proposed that these cytokines act on a CD34⁺ progenitor, generating a CD1a⁺CD14⁻ DC precursor and a CD1a⁻ CD14⁺bipotential (DC/monocyte) precursor, which then undergo terminal differentiation. Similarly, granulocytes, macrophages and DC have been observed in GM-CSF-induced CFU-GM colonies, suggesting these cells arise from a common hematopoietic progenitor. Myeloid-derived DC can be generated in vitro from CD34⁺ progenitor cells isolated from BM, mobilized peripheral blood, or cord blood. Therefore, DC are considered to arise from either myeloid-committed or lymphoid-committed progenitors; however, the specific stages of development within these lineages are poorly defined, largely owing to a lack of understanding of which growth factors regulate this process.

Monocytes are known to differentiate into macrophages with GM-CSF or M-CSF, whereas with GM-CSF and interleukin 4 (IL-4) they differentiate into CD1a⁻ CD14⁺ immature dendritic cells and, with the addition of tumor necrosis factor-α (TNF-α), they differentiate into CD83⁺ mature dendritic cells.

Immature DC have a low expression of co-stimulatory molecules, but are very efficient in antigen uptake and processing. With further maturation, they lose their endocytic capacity and acquire a full repertoire of co-stimulatory antigens and allostimulatory activity. The shift from the immature to the mature stage of differentiation seems to be regulated by TNF-α and the proteins of the TNF superfamily. The phenotypic and functional characterization of DCs has generally been concentrated on mature cells with a high capacity for antigen presentation, while relatively sparse information is available on immature CD34⁺ derived DCs.

The induction of antigen-specific T-cell responses by antigen pulsed dendritic cells and the ability of these cells when injected in vivo to migrate and function as DC is most encouraging. Because of their potent immunostimulatory properties, clinical use of antigen-pulsed DC is being actively pursued and shows initial promise in the treatment of malignancies.

A method for modulating the host's immune response to tumor, microbial, viral, and allergen antigens would provide a key advance in immunotherapy. Immunotherapy for tumors depends on the existence of tumor-specific target antigens. A majority of malignant diseases are not responsive and/or cured using standard therapies and warrant alternative methods of treatment. For example, the idiotype (Id) of the Ig expressed on the surface of non-Hodgkin's lymphoma (NHL) cells is a unique tumor marker. Since these malignancies are monoclonal, all the cells of each tumor produce the same Ig protein. Therefore, these tumor-specific idiotypes can distinguish neoplastic cells from normal cells. Animal studies have shown that active immunization with tumor-derived Id vaccines can induce host immunity. Vaccination with the tumor Ig protein leads to polyclonal antibody and T-cell responses. Such immune responses are capable of recognizing multiple antigenic determinants and, therefore, may prevent the escape of tumor cells with mutations in their idiotypes. These anti-Id responses can protect animals against tumor challenge and can even cure animals with established lymphomas. Therefore, an immunostimulatory molecule and a vaccination strategy that supports strong antigen specific cellular immune responses would be particularly attractive for immunotherapy.

U.S. Pat. No. 5,599,703 discloses a method for the in vitro amplification/expansion of CD34⁺ stem and progenitor cells by culturing those cells on a monolayer of porcine microvascular brain endothelial cells in the presence of cytokines.

Davis et al., CYTOKINE, Vol. 9, No. 4 (1997) pp. 263-275 demonstrated that serum-free medium, conditioned with a mixture of proteins >30 kDa derived from porcine microvascular endothelial cell culture supernatant (PMVEC CM), contained hematopoietic growth factor activity that enhanced the in vitro proliferation, hematopoietic cell production and colony cell formation of primitive human hematopoietic progenitor cells. PMVEC CM can augment the effects in vitro of stem cell factor, interleukin-3, granulocyte-macrophage colony-stimulating factor (GM-CSF), erythropoetin, and granulocyte colony-stimulating factor, all of which are involved in hematopoiesis.

Neutropenia can be the result of disease, genetic disorders, drugs, toxins, and radiation as well as many therapeutic treatments, such as high dose chemotherapy and conventional oncology therapy. Patients suffering from neutropenia are at substantial risk from infection and disease, as the diminished number of neutrophils circulating in the blood substantially impairs the ability of the patient to fight any infection or disease. Treatment of various cancers increasingly involves cytoreductive therapy, including high dose chemotherapy or radiation therapy. These therapies decrease a patient's white blood cell counts, suppress bone marrow hematopoietic activity, and increase their risk of infection and/or hemorrhage. As a result, patients who undergo cytoreductive therapy must also receive therapy to reconstitute bone marrow function (hematopoiesis). Several methods are directed towards restoring the patient's immune system after therapy. Hematopoietic growth factors are administered after therapy to stimulate remaining stem cells to proliferate and differentiate into mature infection fighting cells. Although hematopoietic growth factors can shorten the total period of neutropenia, there remains a critical 10-15 day period immediately following therapy when the patient is severely neutropenic and thus infection prone. Another treatment to manage the problems that result from prolonged bone marrow suppression includes the reinfusion of a patient's own previously harvested peripheral blood precursor cells (PBPC). In such procedures, patients undergo successive treatments with cell mobilization agents to cause mobilization of hematopoietic progenitor cells from the bone marrow to the peripheral circulation for harvesting. Growth factors used for mobilization include interleukin-3 (IL-3), granulocyte colony stimulating factor (G-CSF), granulocyte-macrophage colony stimulating factor (GM-CSF), stem-cell factor (SCF) and a recombinant fusion protein having the active moieties of both IL-3 and GM-CSF (Brandt, S J, et al., N Eng J Med 318:169, 1988; Crawford, J, et al., N Eng J Med 325:164, 1991; Neidhart, J, et al., J Clin Oncol 7:1685, 1989). After harvesting, the patient is given high dose chemotherapy or radiotherapy and the bone marrow function is reconstituted by infusion of the cells harvested earlier.

There is a particular need for agents that stimulate both the in vitro and in vivo development, proliferation and expansion of hematopoietic stem and progenitor cells, committed progenitor cells of the neutrophil and dendritic cell lineages, including neutrophils and dendritic cells. There is a further need in the art for agents that can be used in the simultaneous treatment of cytopenias and anemias such as those caused by destruction of hematopoietic cells in bone marrow such as in the treatment of cancer with chemotherapy and radiation, and pathological conditions such as myelodysplasia, AIDS, aplastic anemia, autoimmune disease or inflammatory conditions. Likewise, there's a need for immunopotentiating cytokines, which can activate/enhance antigen specific immune responses using ex vivo or in vivo techniques. The present invention fulfills these needs and provides other, related advantages.

### SUMMARY OF THE INVENTION

The invention is based upon a variety of surprising and unexpected findings. It has been unexpectedly found that EDHF alone can support the robust and large-scale generation and expansion of tri-lineage pre-dendritic myleomonocytic progenitor cells from hematopoietic stem and progenitor cells. The culture methods disclosed herein show that EDHF (human or porcine proteins) is the only required stimulus and highly purified populations of pre-dendritic myelomonocytic progenitor cells are preferentially generated after 14-21 days of culture in the absence of other cytokines and growth factors. It further has been discovered, unexpectedly that EDHF can be used as a therapeutic agent in vivo to mobilize hematopoietic progenitor cells, expand hematopoietic progenitor cells in various hematopoietic tissues, increase dendritic cell production, and augment host immune responses to vaccine administration and tumor cell challenge. These unexpected results have important utility and therapeutic applications.

Accordingly, objects of this invention are:

1. It's an object of the invention to provide a method for rapidly producing a desired population of pre-dendritic myelomonocytic progenitor cells. Using this method, large populations of pre-dendritic myelomonocytic progenitor cells can be easily, inexpensively and rapidly produced from a very small sample of cells or hematopoictic tissue. Using these methods pre-dendritic myelomonocytic progenitor cell be expanded, stored for later use, stimulated to differentiate or activated or directed to a specific function or target as needed. The procedures described herein are short, straightforward and applicable to hematopoietic progenitor cells from any source. Pre-dendritic myclomonocytic progenitor cells produced by the current invention are useful in immunotherapy applications and provide a number of advantages over current procedures to produce such cells. Further, these methods enable the production of large numbers of pre-dendritic cells, mature dendritic cells, neutrophils and monocytes. The invention further pertains to kits useful in the methods.

2. It's a further object of the invention is to generate ex vivo a substantially pure (>95%) population of genetically modified dendritic cells from CD34⁺ hematopoietic progenitor cells (HPC) that have been modified by artificial introduction of genetic constructs (gene therapy).

3. It's a further object of the invention to describe a methodology that identifies and recovers proteins and genes that are specifically involved in the differentiation and function of dendritic, neutrophil and monocyte cells.

4. It's a further object of the invention to describe a process for the generation of pure populations of dendritic cells that can be used as vaccines in the treatment of infectious diseases and malignancies.

5. It's a further object of the invention, to describe a method for the generation of highly pure populations of dendritic cells that can be used as regulators of unwanted immune responses, such as in autoimmune disease (such as systemic lupus erythematosis), allergic responses and rejection of transplanted organs.

6. Another object of the invention is to describe a method of transplantation therapy wherein primary hematopoietic progenitor cells (e.g. from peripheral blood, bone marrow, cord blood, fetal liver or other tissues) are cultured in vitro with EDHF. The resulting expanded pre-dendritic myelomonocytic progenitor cells are maintained or cryopreserved for later use or can be immediately introduced into a patient for transplantation therapy or other therapeutic or prophylactic uses. Autologous DC cells generated from pre-dendritic myelomonocytic progenitor cells can be used to supplement defective immune systems, repair damaged immune systems or suppress overactive immune systems as a means of treating the associated diseases. Alternatively, DC target cells can be employed to produce useful cell products such as cytokines, lymphokines and chemokines as well as other stimulatory or inhibitory cellular factors.

7. It is a further object of the invention, to describe methods of gene therapy. Pre-dendritic myelomonocytic progenitor cells made by the method of the invention are directly transfected with a genetic sequence or infected with recombinant viral vectors containing a genetic sequence. Cells, which properly express the genetic sequence of interest, are selected, isolated and/or expanded in vitro. Cells expressing the gene of interest are then administered into the patient. Useful genes for gene therapy include genes whose expression products are absent or defective in the patient, and genes and other genetic sequences whose expression provide a beneficial effect to the patient.

8. It is a further object of the invention, to describe a method for stimulating the in vivo immune responses of a mammal, and particular of a human, by isolation, separation, propagation of precursors of DC in high yield from the blood and bone marrow of patients afflicted with various disease states.

9. Another object of the invention is directed to the EDHF composition comprising a mixture of porcine cell proteins having a balance of stimulatory and inhibitory effects favoring the proliferation of hematopoietic stem and progenitor cell populations. The EDHF composition is derived from endothelial cell cultures by isolating soluble proteins having a molecular weight greater than 30 kDa secreted from endothelial cells under serum-free culture conditions. Porcine brain microvascular endothelial cells are the preferred source of endothelial cells.

10. A further object of the invention is to provide a method for stimulating antigen specific immune responses in living cells of a mammal against tumor, microbial, viral and allergen antigens and markers.

11. It is a further object of the invention, to provide a method for inducing mobilization of peripheral blood stem cells (PBSC) from the hematopoietic organs of the body, such as bone marrow, liver, or spleen. The methods involve the administration of an EDHF to a mammal in an amount sufficient to mobilize PBSC.

12. An additional object of the invention is to provide a method for enhancing or facilitating hematopoietic reconstitution or engraftment, by the administration of EDHF. The invention also relates to methods for enhancing progenitor cell mobilization, by administering a growth factor, such as granulocyte colony stimulating factor (G-CSF), in combination with EDHF. The invention further pertains to kits useful in the methods.

13. Another object of the invention is to provide a method for treating a patient suffering from neutropenia, which may result from chemotherapy, conventional oncology therapy, drugs, diseases, genetic disorders, toxins, and radiation, as well as a method of treating a patient who, although not suffering from severe neutropenia, has a reduced population of neutrophils. The method comprises the administration of EDHF and/or pre-dendritic myelomonocytic progenitor cells as described herein.

14. Another object of the invention is to provide a method for treating a subject in order to stimulate hematopoiesis in the subject. The invention involves administering to a subject in need of such treatment an amount of an agent effective to increase the number of hematopoietic cells or mature blood cells in the subject. The present invention relates to methods and compositions for the ex vivo generation of trilineage pre-dendritic myelomonocytic progenitor cells using EDHF.

Briefly, in accordance with the present invention, generation of highly purified tri-lineage pre-dendritic myleomonocytic progenitor cells is achieved by isolating hematopoietic stem and progenitor cells and culturing these cells in the presence of EDHF preferably for between about 14 and 35 days or more whereby tri-lineage pre-dendritic myleomonocytic progenitor cells develop and expand. Preferably, the number of hematopoietic cells are increased at least approximately 10-fold, 20-fold or, most preferably, at least 100-fold relative to the number of hematopoietic cells that are present when the hematopoietic cells initially are contacted with EDHF. Once expanded, lineage specific growth factors are employed to direct differentiation of the tri-lineage pre-dendritic myleomonocytic progenitor cells to the desired mature myeloid cells such as dendritic cells, macrophages and neutrophils. Advantageously, the methods of the invention do not require the presence of additional cytokines to support the stimulation of hematopoietic cells in vitro. Accordingly, the methods and compositions of the invention are useful for increasing the number of pre-dendritic myelomonocytic hematopoietic cells on a large-scale basis with significant cell purity (>95% DC) followed by lineage specific differentiation culture steps. Large-scale generation of such rare cells under ex vivo culture conditions from hematopoietic and progenitor cells in culture permits the characterization of such cells in culture under a variety of conditions, as well as the use of such cultured cells and material made from said cells for the isolation/identification of naturally occurring molecules therefrom in vitro. A major goal is to expand and terminally differentiate pre-dendritic myelomonocytic hematopoietic cells (unmodified or genetically modified) ex vivo into mature functional dendritic cells using DC specific differentiation agents (i.e., GM-CSF+IL-4+TNF-α, CD40L, LSP, etc), charge these purified DCs with antigens, and reinfuse them to enhance host resistance. A major goal is to use this methodology to discover and identify key genes and proteins involved in DC, neutrophil and monocytes differentiation and function.

The present invention employs methods and compositions (EDHF) used as a therapeutic agent to stimulate hematopoiesis, hematopoietic progenitor cell expansion, and dendritic cell development in vivo. Moreover, pretreatment of a mammal with EDHF can be used in vivo as an immunopotentiating agent to enhance the effectiveness of vaccines. Of particular usefulness in a clinical setting is the expansion and activation of mature myeloid dendritic cells to increase the effectiveness or potency of vaccines by increasing antigen uptake and antigen presentation to other immune cells. This is an important treatment strategy for increasing the effectiveness of vaccines because the antigen(s) do not have to be modified and the dendritic cells do not have to be expanded and purified in vitro and then engrafted into the patient. Therefore, other applications of this invention include therapies aimed at mobilizing hematopoietic progenitor cells, accelerating hematopoietic engraftment and in boosting anti-tumor and other desirable immune responses.

### BRIEF DESCRIPTION OF THE DRAWINGS

FIG. 1A is a dose titration curve comparing EDHF with other cytokines. FIG. 1B shows the growth of cord blood CD34⁺ cells (2×10⁵) using EDHF as the only source of growth factors. Immunomagnetic selected cord blood CD34⁺ cells were cultured for 35 days in medium supplemented with an optimal concentration of EDHF. At day 7 and at weekly intervals thereafter, the cultures were subjected 1 to 5 in splits and refed with fresh medium containing EDHF. Data points represent average cell expansion (fold increase above input cell number), which was calculated from the number of cells produced at each time interval and the dilution factor. The results of 8 separate experiments are expressed as fold increase in nucleated cell counts (mean+standard deviation of the mean [SD]) performed using cells from 8 different donors.

FIG. 2 shows representative photographs of Wright-Giemsa-stained cytospin preparations of purified CD34⁺ cord blood cells and cells cultured with EDHF after 7-35 days (original magnification×500). Arrows indicate cells that display mitotic figures.

FIG. 3 shows cells generated in culture from cord blood CD34⁺ cells treated with EDHF for 21 days have the capacity to differentiate into DC-like cells. Cord blood CD34⁺ cells were expanded for 21 days in the presence of EDHF alone. (FIG. 3A) Cells were then replated in identical medium containing GM-CSF+IL-4+TNF-α and photographed in situ (×200 magnification using phase contrast lens) 3-4 days later. (FIGS. 3B & 3C) DC from culture were harvested, re-suspended in fresh medium and transferred onto glass microscope slides, coverslipped, immediately examined microscopically and photographed (×500 and ×200 magnification). FIG. 3D is a photograph of a Wright-Giemsa stained cytocentrifuge cell preparation of generated DC (×500 magnification). FIG. 3E shows the total nucleated cell yield per culture condition (note y-axis is log scale). FIG. 3F shows the total number of cells at day 28 of culture expressing immature and mature DC phenotypes (see FIG. 4 for frequency results). Results are representative of 8 different experiments.

FIG. 4 shows the flow cytometeric analysis of immature DC precursors derived from CD34⁺ cord blood CD34⁺ cells cultured for 21 days in EDHF alone followed by 3-4 days of culture in identical medium containing GM-CSF+IL-4+TNF-α double stained with a panel of PE-conjugated and FITC-conjugated mAbs. Stained cells were analyzed on a FACScan (Becton Dickinson) and the percentage of total gated events is indicated in each quadrant. Quadrants were set to include 99% of cells stained with isotype control antibodies in lower left quadrants. Data shown are representative of three independent experiments with 3 different cord blood samples. FIG. 4B shows fluorescent images of EDHF derived mature dendritic cells.

FIG. 5 is a flow diagram that shows that EDHF supports the expansion of hematopoietic progenitor cells capable of multilineage differentiation. Cord blood cells were cultured with EDHF for 14 days, harvested, washed and then re-cultured in fresh culture medium supplemented with either M-CSF (20 ng/mL), G-CSF (20 ng/mL), GM-CSF (20 ng/mL), GM-CSF+IL-3, or GM-CSF+IL-4 (20 ng/mL)+TNF-α (20 ng/mL) for an additional 7 days. Representative photographs of cells generated under each culture condition are depicted (×500 magnification).

FIG. 6 shows photographs of multiple stages of DC differentiation from EDHF derived DC precursor cells. Cells generated in culture from cord blood CD34⁺ cells treated with EDHF for 21 days have the capacity to differentiate into cells that exhibit the Langerhan cell (LC) or DC morphology in culture. Cord blood CD34⁺ cells were expanded for 14 days in the presence of EDHF alone. Cells were then re-plated in identical medium containing GM-CSF+IL-4+TNF-α cultured for an additional 3-10 days. (FIGS. 6A & 6B) After 3-4 days of culture, plastic adherent cells (panel A &B) were stained with Wright-Giemsa photographed in situ (×100 and ×200 magnification, respectively). (FIG. 6C) Cytocentrifuge cell preparations of the nonadherent cell population from the same cultures were made, fixed, stained with Wrights-Giemsa and then photographed (×500 magnification). (FIG. 6D) After 7-10 days of DC induction using the combined treatment of GM-CSF+IL-4+TNF-α, all cells are nonadherent and exhibit the typical DC morphology with a corona of numerous dendritic processes evident on each cell.

FIG. 7 is a comparative analysis of EDHF versus the combination of SCF+FLT3+GM-CSF treatment on the ex vivo expansion of DC precursors using purified cord blood CD34⁺ cells. CD34⁺ cord blood cells were cultured for 7 days with optimal concentrations of EDHF alone or SCF+FLT3L+GM-CSF followed by 7 days of culture in identical medium in the presence or absence of GM-CSF+IL-4+TNF-α. (FIG. 7A) Cultured cells at day 7 and 14 of culture were harvested from culture, washed, and manual hematocytometer cell counts performed using trypan blue dye exclusion. (FIG. 7B) Cells collected at day 14 of culture, were double stained with a FITC-conjugated HLA-DR⁺ mAb and PE-conjugated CD83 mAb (see FIG. 10). The percent HLA-DR⁺CD83⁺ cells in each culture is illustrated in FIG. 7B. FIG. 7C shows the total number of HLA-DR⁺CD83⁺ cell generated under each culture condition. The data represents average cell expansion (fold increase above input cell number), which was calculated from the number of cells produced at each time interval and the HLA-DR⁺CD83⁺ cell frequency. FIG. 7D is a photograph from a Wright-Giemsa stained cytocentrifuge cell preparation of cells expressing DC characteristics (×500 magnification). Note: all induced DC cells had similar morphological appearance independent of the initial culture condition.

FIG. 8 contains two photographs that confirm that EDHF supports the expansion of hematopoietic progenitor cells that exhibit no morphological DC-like appearance. Representative phase contrast microscopic appearance of day 21 cultures in situ (FIG. 8A, phase contrast lens, ×200 magnification) and Wright-Giemsa stained cytocentrifuge cell preparation of harvested cells (FIG. 8B, ×500 magnification).

FIGS. 9 and 9A shows the results of flow cytometeric analysis of immature DC precursors derived from CD34⁺ cord blood cells cultured for 7 days in optimal concentrations of EDHF alone or SCF+FLT3L+GM-CSF followed by 7 days of culture in identical medium in the presence or absence of GM-CSF+IL-4+TNF-α. Cells were harvested from culture, washed, counted and double stained with a FITC-conjugated HLA-DR mAb and PE-conjugated CD83 mAb. Stained cells were analyzed on a FACScan (Becton Dickinson) and the percentage of total gated events is indicated in each quadrant. Quadrants were set to include 99% of cells stained with isotype control antibodies in lower left quadrants.

FIG. 10 shows that ex vivo generated DC derived from cord blood CD34⁺ cells treated with EDHF are strong stimulators of primary MLR T cell responses. CD34⁺ cord blood cells were cultured for 7 days in optimal concentrations of EDHF alone or SCF+FLT3+GM-CSF followed by 7 days of culture in identical medium in the presence or absence of GM-CSF+IL-4+TNF-α. After 21 days of culture, 5×10⁴ allogeneic CD4⁺ T cells were incubated with graded doses of mytomycin-C cultured APC. CD34⁺ cells were also incubated with EDHF treated cells that were additionally cultured with GM-CSF (20 ng/mL) for the last 7 days. MLR cultures were incubated for 7 days at 37° C. in a 5% CO2 in air humidified atmosphere. Proliferation of CD4⁺ T cells induced by allogeneic mytomycin-C treated APC was measured using the Alamar blue assay and results are expressed as absorbance 570 nm. FIG. 10A shows MLR response at all graded doses of APC whereas results in FIG. 10B depict the results at half maximal levels (T:DC ratio of 320:1) for the most potent APC. One experiment representative of two is shown.

FIG. 11 shows that EDHF is far superior to the previously reported combination of SCF+FLT3L+GM-CSF in supporting the ex vivo expansion of DC precursors from purified cord blood CD34⁺ cells. Highly purified CD34⁺ cord blood cells were cultured for 14 days with optimal concentrations of EDHF alone, SCF+FLT3L+TPO or EDHF+SCF+FLT3L+TPO followed by an additional 7 days of culture in identical medium in the presence or absence of GM-CSF+IL-4+TNF-α. (FIG. 11A) Generated cells at day 21 of culture were harvested, washed, and manual hematocytometer cell counts performed using trypan blue dye exclusion. (FIG. 11B) Cells collected at day 21 of culture were double stained with either FITC-conjugated CD14 mAb and PE-conjugated CD1a mAb or FITC-conjugated HLA-DR⁺ mAb and PE-conjugated CD83 mAb (see FIG. 12 for phenotype frequency). Total number of CD14-CD1a⁺ and HLA-DR⁺CD83⁺ cells generated under each culture condition as shown in FIG. 11B. The data represents the average cell expansion (fold increase above input cell number), which was calculated from the number of cells produced at each time interval and the HLA-DR⁺CD83⁺ cell frequency.

FIGS. 12A-C show the results of flow cytometeric analysis of immature DC precursors derived from CD34⁺ cord blood cells cultured for 14 days in optimal concentrations of EDHF alone or SCF+FLT3L+TPO followed by 7 days of culture in identical medium in the presence or absence of GM-CSF+IL-4+TNF-α. Cells were harvested from culture, washed, counted and double stained with a panel of PE-conjugated and FITC-conjugated mAbs. Stained cells were analyzed on a FACScan (Becton Dickinson) and the percentage of total gated events is indicated in each quadrant. Quadrants were set to include 99% of cells stained with isotype control antibodies in lower left quadrants.

FIG. 13 shows the effects of EDHF on HPC mobilization in mice. Mice were administered EDHF or control vehicle for 7 days. On day 8, mice were killed and WBC counts (FIG. 13A), and CFC numbers in the blood (FIG. 13B) measured as described in Example 2. The data represent the mean±1SD.

FIG. 14 shows the changes in bone marrow cellularity (FIG. 14A) and bone marrow CFC numbers (FIG. 14B) in mice treated with EDHF or control vehicle for 7 days. CFC measurements were performed as described in Example 2. A total of 10 mice were analyzed for each data point. The data represent the mean±1SD.

FIG. 15 shows the changes in spleen weight (FIG. 15A), spleen cellularity (FIG. 15B) and spleen CFC (FIG. 15C) numbers in mice treated with EDHF or control vehicle for 7 days. CFC measurements were performed as described in Example 2. A total of 5 mice were analyzed for each data point. The data represent the mean±1SD.

FIG. 16 shows that EDHF administration increases the frequency of DC cells in the spleen. Splenocytes isolated from control and EDHF treated mice were analyzed by FACS for CD11 c⁺IA/I-E⁺ CD86⁺ cells following 7 days of treatment. Contour plots represent IA-e versus CD11c profiles and CD11c versus CD86 profiles. The lower left-hand quadrant represents the fluorescence obtained with isotype control matched antibodies.

FIG. 17 shows the increase in absolute number of splenic DC following EDHF administration. The absolute number of splenic CD11c⁺ IA/I-E⁺ CD86⁺ in control and EDHF treated mice was calculated by multiplying the total cell count by the percentage of CD11c⁺ IA/I-E+CD86⁺ cells. A total of 5 mice were analyzed for each data point. The data represent the mean±1SD.

FIG. 18 shows anti-38C13 titers in vaccinated animals. Ten days after the first vaccination (panel A) and ten days after the second vaccination (panel B), serum from each group of 10 C3H/HEN mice vaccinated with 38C13 scFv in the absence or presence of the adjuvant cpG DNA was pooled and analyzed with the native 38C13 IgM as the target. Anti-38C13 IgG1 and IgG2a serum levels were quantitated by comparison with purified isotype-specific mouse anti-38C13 standards. Concentrations of IgG1 and IgG2a are shown for all groups. No detectable anti-38C13 was measured in the control vaccine groups.

FIG. 19 shows mice pretreated with EDHF and immunized with plant-derived scFv protein plus cpG DNA are protected from tumor challenge. Tumor protection was measured from the time of tumor implantation (day 0) and is plotted as percent survival. These results are representative of two experiments. While all vaccinated groups statistically differed from the susceptible control (P<0.00001), there was no statistical segregation among vaccinated groups.

FIG. 20: illustrates the experimental design to assay for expansion human SCID/NOD repopulation cells (SRC).

FIG. 21: shows the morphology of cells derived from CD34⁺ cord blood cells treated for 7-days with EDHF. Cells were transplanted into SCID/NOD mice.

FIG. 22: shows the effect of EDHF treatment on human cell engraftment in the bone marrow of NOD/SCID mice.

FIG. 23: shows the effect of EDHF treatment on the distribution and frequency of human hematopoietic progenitor cells in the bone marrow of SCID/NOD mice.

FIG. 24: shows the level of total human progenitor cell engraftment in a single SCID/NOD mouse femur.

FIG. 25 shows that the day 14 colony-cell morphology of human progenitor cell cultured from chimeric SCID/NOD bone marrow.

FIG. 26 shows that day-14 colony-forming cells cultured from chimeric mouse SCID/NOD mouse bone marrow were derived from expanded and transplanted human CD45⁺ hematopoietic stem and progenitor cells.

FIG. 27 shows the effect of EDHF on PMVEC in combination with complete endothelial cell culture medium containing 10% heat-inactivated fetal bovine serum.

FIG. 28 shows the effect of various concentrations of EDHF on the growth of PMVEC, BPEC-3736 clone-1, and HUVEC clone 082901) in combination with complete endothelial cell culture medium containing 10% heat-inactivated fetal bovine serum.

FIG. 29 shows the effects of EDHF (100 μg/mL) on the growth of PMVEC under both serum-free and serum containing culture conditions.

FIG. 30: shows the effect of various concentrations of EDHF on the growth of PMVEC (panel A) and HUVEC clone 082901(panel B) in combination with serum-free human endothelial cell culture medium.

### DESCRIPTION OF THE PREFERRED EMBODIMENTS

The following terms used herein are defined below.

Definitions

Activation is a process by which genetic and phenotypic adaptation of a cell takes place resulting in new cellular functionalities. A cell that is “activated” from a quiescent or non-proliferative state in response to a signal, such as a cytokine, a growth factor or an antigen, enters a more functional or “active” state relative to other cells of the same lineage. Functional states resulting from activation include proliferation, antigen presentation and processing, intercellular signaling, inflammatory responses, etc.

Allogeneic Stem Cell Transplantation is the transfer of stem cells from one person, the donor, to another, the recipient who is not an identical twin. In practice, one makes an effort to find a donor who is very similar in tissue type to the recipient by matching their HLA types. The closer the similarity the higher the probability that the transplant will be a success and that harmful immune reactions will be minimized. Siblings are the most likely to be closely matched, but other family members and unrelated matched donors can be similar enough to achieve a successful transplant if the optimal match is not available and the severity of the illness justifies the risk. In the treatment of leukemia, lymphoma, and myeloma, the cells to be transplanted are pluripotential stem cells, but they might be admixed with other marrow or blood cells when infused.

Autologous Stem Cell Infusion is a technique, often referred to as transplantation, which involves 1) harvesting the patient's stem cells from blood or marrow, 2) freezing them for later use, and 3) thawing and infusing them via an indwelling catheter after the patient has been given intensive chemotherapy or radiation therapy. The blood or marrow may be obtained from a patient with a disease of the marrow (for example, acute myelogenous leukemia) when in remission or when the marrow and blood is not overtly abnormal (for example, lymphoma). Technically, this procedure is not transplantation, which implies taking tissue from one individual (donor) and giving it to another person (recipient). The purpose of this procedure is to restore blood cell production from the preserved and reinfused stem cells after intensive therapy has severely damaged the patient's remaining marrow. This procedure can be performed using marrow or blood stem cells. The latter can be harvested by hemapheresis

CD14⁺ peripheral blood monocytes: Monocytes/macrophages found in the peripheral blood which express CD14, the receptor for endotoxin (lipopolysaccharide [LPS]). When LPS binds to CD14 expressed by monocytes or macrophages, the cells become activated and release cytokines such as tumor necrosis factor α (TNF-α) and up-regulate cell surface molecules, including adhesion molecules.

CD34 molecule is a monomeric type I integral trans-membrane glycoprotein of apparent molecular weight 105-120 kDa. The 373 amino-acid protein backbone (40 kDa) is 105 heavily glycosylated with a maximum of 9 complex-type N-glycans and numerous highly sialylated O-linked glycans. This glycosylation pattern is characteristic of the sialo-mucin family, which comprises leucosialin and CD43. A key issue in CD34 biochemistry is the polymorphism of glycosylation, as demonstrated by epitope variability in immunological analysis. The gene coding for the CD34 antigen is located on chromosome region 1q32, in a region containing a cluster of genes encoding adhesion molecules. However, the CD34 amino acid sequence shows no identified homology with any known protein. The function of the CD34 antigen in early hematopoiesis still remains elusive. The mucin-like structure of CD34 suggests a role in cellular adhesion, possibly the stromal cells.

CD34⁺ hematopoietic progenitor cells (HPC) constitute only a small fraction of the hematopoietic tissue detected in bone marrow and peripheral blood as well as in fetal hematopoietic tissue and umbilical cord blood. This cellular compartment is in fact heterogeneous, comprising extremely primitive stem cells (quiescent stem cells with self-renewal and repopulation capabilities) and multilineage progenitor cells at various stages of differentiation and maturation.

CD34⁺lin⁻(CD34⁺, CD38⁻, HLA-DR⁻, CD90 (Thy-1)⁺ cell) phenotype defines a subset of CD34⁺ progenitor cells found in the blood, cord blood, bone marrow and fetal hematopoietic tissue which contains the most primitive cells. Acquisition of CD38 expression and loss of CD90 expression occurs with lineage-commitment and differentiation of progenitor cells. Differentiation can be further assessed with the expression of lineage-specific antigens such as CD33, CD13, CD7, CD10, CD19, CD56, CD41a, and Glycophorin A.

CD80 is the new designation of the natural ligands of CD28, a 44-54 kDa glycoprotein, called B7, BB1. CD80 is a membrane glycoprotein of 262 amino acids, which is expressed primarily on activated B-cells and other antigen-presenting cells. It is expressed by macrophages, keratinocytes, T-cells, B-cells, peripheral blood dendritic and Langerhans cells.

CD83 is currently one of the best cell surface markers for human mature dendritic cells. The cell surface antigen is found expressed on non-follicular dendritic cells, circulating dendritic cells, interdigitating dendritic cells within lymphoid tissues, Langerhan's cells, in vitro generated dendritic cells, dermal cells, and thymic dendritic cells.

CD86 also designated B7-2 (306 amino acids) is another ligand for CD28 and is found on blood dendritic and Langerhans cells, B-cells, macrophages, Kupffer cells, activated monocytes and various natural killer cell clones. Binding of the B7s to CD28 on T-cells delivers a costimulatory signal that triggers T-cell proliferation by stimulating a transcription factor that, in turn, induces the synthesis and secretion of IL2 and other cytokines.

Dendritic cells are a system of professional antigen-presenting cells that initiate the immune responses. Dendritic cells are widely distributed in the body, both in non-lymphoid tissues, lymphoid tissues and fluids of the body. Dendritic cells arise from CD34⁺ bone marrow progenitor cells and can be classified into interstitial dendritic cells in non-lymphoid tissues, interdigitating dendritic cells in secondary lymphoid tissue, dendritic cells in blood and veiled cells in lymphatics. They can exhibit differences in each of these compartments that relate to maturation state and microenvironment. Dendritic cells process and present antigens efficiently in situ and stimulate responses from naive and memory T cells in the paracortical area of secondary lymphoid organs. Properties contributing to the dendritic cells' specialized function are the efficiency in clustering T cells and giving the right signals needed to activate naive and resting T cells.

Differentiation is a term intended to have its ordinary meaning in the art of cell development from a primitive to specialized state.

Expanded means elevated in number. The cells are expanded by cultivating them in an appropriate growth medium with a growth factor such as EDHF.

EDHF means Endothelial Cell Derived Hematopoietic Growth Factor comprising one or more animal endothelial cell derived hematopoietic growth factor proteins having a molecular weight (MW) greater than about 30 kDa and having the associated biological activity.

Hematopoiesis is the bodily process of producing both red and white blood cells from their progenitor stem cells in the marrow. The most undeveloped cells in the marrow are stem cells. They start the process of blood cell development. The stem cells begin to develop into young or immature blood cells like red cells or white cells of various types. This process is called “differentiation.” The young or immature blood cells then further develop into fully functional blood cells. This process is called “maturation.” The cells then leave the marrow, enter the blood, and circulate throughout the body. Hematopoiesis is a continuous process that is active normally throughout life. The reason for this continuous activity is that most blood cells live for short periods and must be continuously replaced. After release from the marrow, red cells are removed in four months, platelets in 10 days and most neutrophils in one to three days. About five hundred billion blood cells are made each day. This requirement for very rapid replacement explains the severe deficiency in blood cell counts when the marrow is injured by replacement with leukemia, lymphoma or myeloma cells.

Langerhan cells are antigen-presenting cells of the skin, which emigrate to local lymph nodes to become dendritic cells; they are very active in presenting antigens to T cells.

Hematopoietic tissue include bone marrow, fetal liver, spleen, peripheral blood, cytokine mobilized stem cells or umbilical cord blood and the like.

Hematopoietic stem cell and progenitor cells. The pluripotent hematopoietic stem cell can be defined functionally as well as phenotypically. Functionally, stem cells are those hematopoietic cells having the capability for prolonged self-renewal (generate daughter cells identical to mother cells) as well as the ability to differentiate into all the lymphohematopoietic cell lineages. Thus pluripotent hematopoietic stem cells, when localized to the appropriate microenvironment, can completely and durably reconstitute the hematopoietic and lymphoid compartments. Multilineage stem and progenitor cells can also be identified phenotypically by cell surface markers. A number of phenotypic markers, singly and in combination, have been described to identify the pluripotent hematopoietic stem cell. Primitive human stem cells have been characterized as small cells that are CD34⁺CD38⁻, HLA-DR⁻, Thy1^{+/-}, CD15⁻, Lin⁻, c-kit⁺, 4-hydroperoxycyclophosphamide-resistant and rhodamine 123 dull. Equivalent primitive murine stem cells have been characterized as Lin⁻, Sca⁺, and Thy1.1⁺. Preferably, the human CD34⁺ stem cells used in the present culture system are a subset of the CD34⁺CD38⁻ cell population.

Hematopoictic progenitor cells. Committed and/or differentiated cells and cycling CD34⁺ cells that express the activation cell surface marker antigen CD38 in addition to CD34. Hematopoietic progenitor cells can also express other antigen markers specific for the myeloid lineage such as HLA-DR and Lin. Hematopoietic progenitor cells are derived from CD34⁺ CD38⁻ stem cells, are capable of limited self-renewal and differentiation, and provide relatively short-term hematopoietic reconstitution in vivo.

Tri-lineage pre-dendritic myleomonocytic progenitor (precursor) cells. Hematopoietic cells that are committed multi-lineage myeloid progenitor cells with limited proliferation potential that possess the ability to differentiate into mature populations of neutrophils, monocytes or and/or dendritic cells when stimulated with lineage specific growth factors.

The term “CD34⁺ stem and progenitor cells” when used herein with respect to human hematopoietic cells refers to a mixed population of CD34⁺ CD38⁻ and CD34⁺CD38⁺ hematopoietic progenitor cells as described herein. Also, any reference to embodiments relating to human hematopoietic cells such as CD34⁺ cells equally encompasses the same embodiment with a non-human mammal. It is well understood by one of skill in the art that various species have their own set of specific marker proteins.

Hematopoietic Stem and Progenitor Cells

The hematopoietic stem and progenitor cells used in the present culturing method can be isolated from various hematopoietic tissues such as adult bone marrow, fetal liver, spleen, peripheral blood, cytokine mobilized stem cells or umbilical cord blood using methods known in the art. The present culturing method is useful for amplifying/expanding mammalian stem and progenitor cells from various species. Preferred species include humans, non-human primates and mice. The stem and progenitor cells utilized in the present method are preferably substantially enriched, that is, depleted of mature lymphoid and myeloid cells. In the case of human hematopoietic cells, the CD34⁺ stem and progenitor cells are enriched at least 85%, more preferably at least 95%, and most preferably at least 99%. Several methods by which CD34⁺ stem and progenitor cells can be isolated and enriched to high degrees of purity using positive immunoselection have been described by Berenson et al (Journal of Immunological Methods, 91:11-19, 1986), Thomas et al (Prog Clin Biol Res 377:537-44, 1992), and Okarma et al (Prog Clin Biol Res 377:487-502, 1992). Immunomagnetic enrichment can also be employed according to known procedures as described in Example 1 below.

As used herein, human hematopoietic cells include hematopoietic stem cells, primordial stem cells, early progenitor cells, CD34⁺ cells, early lineage cells of the mesenchymal, myeloid, lymphoid and erythroid lineages, bone marrow cells, blood cells, umbilical cord blood cells, stromal cells, and other hematopoietic precursor cells that are known to those of ordinary skill in the art.

EDHF

The endothelial cell derived hematopoietic growth factors (EDHF) comprise one or a mixture of more than one animal cell proteins having biological activity inducing proliferation and/or differentiation of hematopoietic stem and progenitor cell populations. The EDHF composition is derived from endothelial cell cultures or culture medium supernatant alone or by fractionating or isolating one or more proteins. These proteins preferably have a molecular weight of greater than about 30 kDa and more preferably having a molecular weight of from about 30 kDa to about 100 kDa and more preferably from about 50 kDa to about 80 kDa. The mixture of proteins may be collected from these endothelial cells or their culture supernatant under serum-free culture conditions. Alternatively, the individual protein(s) may be independently synthesized, preferably by expression of the corresponding gene(s) in suitable recombinant host(s) by conventional means.

The EDHF may be isolated from animal, preferably human or porcine, endothelial cell cultures and the proteins can be soluble proteins or membrane bound proteins or active fragments of expressed endothelial cell proteins. Porcine brain microvascular endothelial cells are the preferred source of endothelial cells. The EDHF is preferably isolated from the supernatant obtained from human or porcine endothelial cells grown under serum-free culture conditions employing standard and routine isolation and separation techniques. For instance, the supernatant is placed on a separation membrane and pressure is applied to separate the lower fraction molecular weight compounds from the higher molecular weight fraction. The particular size of the fractions depends on the pore size of the separation membrane employed. One technique for obtaining EDHF from porcine brain microvascular endothelial cells is described in Davis, et. Al., CYTOKINE, Vol. 9, No. 4 (April), 1997: pp 263-275 which is incorporated herein by reference. Examples of other human or porcine endothelial cells suitable for use obtaining EDHF according to the present invention include, but are not limited to, microvascular endothelial cells, brain endothelial cells, and various types of immortalized endothelial cells.

A number of other fractionation procedures may be used to obtain fractions with the desired activity to use a more purified product and to avoid possible without unwanted side effects. The particular protein factor(s) responsible for each biological activity may be purified and used or identified and then artificially synthesized. Because different biological activities have been observed with EDHF, it is likely that different proteins or combination of proteins are responsible for some of the different activities.

In one embodiment of the present invention, human endothelial cells are used as the source of EDHF wherein a mixture of proteins >30 kDa are isolated from the supernatant of human endothelial cell cultures similar to the procedures described herein for preparing porcine EDHF. In another embodiment one or more Insulin-Like Growth Factor Binding Proteins (IGFBP), which are expressed in endothelial cells are employed as growth factors as described herein. A preferred IGFBP is IGFBP-3 having a molecular weight of about 53 kDa. When IGFBP are employed as the EDHF then the IGFBP proteins can be made by recombinant technology thereby eliminating the need to isolate the EDHF from the endothelial culture medium or supernatant.

EDHF may be of cellular nature with the actual endothelial cells being used in adjacent coculture with a common supernatant or even direct contact with the target cells (e.g. stem cells) one wishes to proliferate or differentiate. Cellular EDHF is generally less desirable than acellular EDHF derived from extracts and proteins as described above, because of the need for a cell-cell separation method rather than a simpler cell-liquid separation. Since cellular contact provides all of the interactions of acellular supernatant contact and additional interactions, one may reasonably conclude that biological activities observed in acellular EDHF will be similar for the corresponding cellular EDHF. However, the reverse cannot be concluded short of experimental data because cellular EDHF may have additional biological activities not shared with acellular EDHF due to membrane bound proteins, cellular matrix and other contact-dependant biological activities.

In Vitro Applications

The invention provides methods for the optimal ex vivo generation of tri-lineage pre-dendritic myelomonocytic progenitor cells. Hematopoietic progenitor cells originate either from bone marrow, from cord blood, or from peripheral blood. Bone marrow samples may be obtained either from normal donors or from patients. Umbilical cord blood is obtained after normal gestations. Peripheral blood is obtained either from normal donors or from patients. In some cases, patients are treated with EDHF or other described hematopoietic growth factors to “mobilize” or stimulate their stem cells to move from bone marrow to their peripheral blood stream, thus greatly increasing the number of stem/progenitor cells in their peripheral blood samples. Mononuclear white blood cells (leukocytes) are first separated from the samples of bone marrow or cord or peripheral blood by standard methods such as centrifugation through a gradient. Next, an enriched population of hematopoietic progenitor cells are collected utilizing separation procedures which may include magnetic separation, using antibody-coated magnetic beads, affinity chromatography, cytotoxic agents, either joined to a monoclonal antibody or used in conjunction with complement, and “panning”, which utilizes a monoclonal antibody attached to a solid matrix, or other convenient techniques. Preferably, stem/progenitor cells are separated from other cell types by the cell-surface expression of CD34. For example, CD34⁺ cells may be positively selected by magnetic bead separation, wherein magnetic beads are coated with CD34-reactive monoclonal antibody as described in Example 1 below. In the present culture system, the enriched CD34⁺ stem and progenitor cells are placed in direct contact with EDHF generally for between about 14 and 35 or more days and preferably between about 14 and about 21 days. Preferably, no additional early growth factors, such as TPO, Flt3 ligand and SCF, are employed during this culture period. During this culture period the hematopoietic stem and progenitor cells proliferate, expand, and give rise to a population of cells highly enriched for pre-dendritic myelomonocytic progenitor cells. Prior to 21 days of culture, few if any monocyte or granulocyte related cells are detected as indicated by a substantial absence of CD14⁺ and CD86⁺ expressing cells. Cells cultured according to the present invention possess myeloid colony forming potential. After the tri-lineage pre-dendritic myleomonocytic progenitor cells are generated, they can be further cultured with one or more late acting growth factors that promote differentiation into mature cells. Late acting growth factors include IL-3, IL-6, GM-CSF, M-CSF, G-CSF, IL-1, TNF-α, GM-CSF/IL-3 fusions, IL-4, TPO and others.

Positively selected stem and/or progenitor cells are placed in culture at densities ranging from 10,000 to 200,000 cells/mL, preferably at 50,000 cells/mL. Any standard tissue culture flasks, petri dishes, containers or bags may be used in either a static or a perfusion culture system (Koller, M R, et al., BIO/TECHNOLOGY 11:358-363; Emerson, S G, et al., PCT WO92/11355). When a static culture system is used, the cells are fed fresh complete hematopoietic culture medium, as detailed in Example 1 below, containing EDHF at intervals of 5 to 7 days to replenish nutrients and remove wastes. Cell densities at 5-7 day intervals are adjusted to 0.1-2.5×10⁶ cells/mL. A cell density of 0.5×10⁶ cells is preferred. The EDHF is added to the culture media in amounts effective to expand the predendritic myleomonocytic progenitor cells. A preferred range of EDHF in media is from about 1 to about 50 μg/L with a concentration of about 10 μg/L being particularly preferred.

Isolated stem and progenitor cells can be frozen in a controlled rate freezer (e.g., Cryo-Med, Mt. Clemens, Mich.), then stored in the vapor phase of liquid nitrogen using dimethylsulfoxide as a cryoprotectant. A variety of growth and culture media can be used for the growth and culture of dendritic cells (fresh or frozen), including serum-depleted or serum-based media. Useful growth media include RPMI, TC 199, Iscove's modified Dulbecco's medium (Iscove, et al., F. J. Exp. Med., 147: 923 (1978)), DMEM, Fischer's, alpha medium, NCTC, F-10, Leibovitz's L-15, MEM and McCoy's. A preferred culture media protocol is described in Example 1 below.

Commercially available serum-free media formulations may also be utilized provided they are supplemented with human albumin and the requisite growth factors. Particular nutrients present in the media include serum albumin, L-glutamine, transferrin, lipids, cholesterol, a reducing agent such as 2-mercaptoethanol or monothioglycerol, pyruvate, butyrate, and a glucocorticoid such as hydrocortisone 2-hemisuccinate. The standard media includes an energy source, vitamins or other cell-supporting organic compounds, a buffer such as HEPES, or Tris, that acts to stabilize the pH of the media, and various inorganic salts. A variety of serum-free cellular growth media are described in WO 95/00632, which is incorporated herein by reference.

In one embodiment the collected CD34⁺ cells are cultured with EDHF, as described herein, and allowed to differentiate and commit to cells of the dendritic lineage. These cells are then further purified by flow cytometry or similar means, using markers characteristic of dendritic cells, such as CD1a, HLA-DR, CD80 and/or CD86. The cultured dendritic cells can then be exposed to an antigen, for example, a tumor antigen or an antigen derived from a pathogenic or opportunistic organism, allowed to process the antigen, and then cultured with an amount of a CD40 binding protein to activate the dendritic cell. Alternatively, the dendritic cells can be transfected with a gene encoding an antigen, and then cultured with an amount of a CD40 binding protein to activate the antigen-presenting dendritic cells. The activated, antigen-carrying dendritic cells are then administered to an individual in order to stimulate an antigen-specific immune response. The dendritic cells can be administered prior to, concurrently with, or subsequent to, antigen administration. Alternatively, T cells may be collected from the individual and exposed to the activated, antigen-carrying dendritic cells in vivo to stimulate antigen-specific T cells, which can then be administered to the individual.

The present invention also includes the generation of mature hematopoietic cells by culturing CD34⁺ stem and progenitor cells with EDHF as described herein and preferably for about 21 days. At the 21 day time period other late acting growth factors are added to the culture media to direct differentiation into the desired mature hematopoietic cells. The pre-dendritic myelomonocytic progenitor cells also may be used in the research of the proliferation and differentiation of these cells into linage specific mature cells (i.e., neutrophils, monocytes and dendritic cells). For example, factors associated with proliferation and differentiation, such as hematopoietic growth factors, may be evaluated. In addition, cytokine combinations and extracellular conditions may be evaluated. Similarly, the cells may be used to discover, identify, isolate and recover proteins and genes that are specifically involved in the differentiation and function of dendritic, neutrophils and monocytes cells. The pre-dendritic myelomonocytic progenitor cells may be frozen in liquid nitrogen for long periods of storage. The cells then may be thawed and used as needed. Typically, the cells may be stored in 10% DMSO, 50% Serum, and 40% RPMI 1640 medium. Once thawed, the cells may be induced to proliferate and further differentiate by the introduction of the appropriate hematopoietic growth factors.

Given the disclosure herein, it will be apparent to one of skill in the art that growth factor combinations and concentrations may be manipulated to yield various results. For example, G-CSF and GM-CSF will enhance the production of neutrophils whereas M-CSF will enhance the production of monocytes, and GM-CSF+IL-4+TNF-α will enhance the production of dendritic cells.

Ex vivo generated pre-dendritic myelomonocytic progenitor cells may also find use in the treatment of neutropenia induced by a disease, drug, toxin or radiation, as well as genetic or congenital neutropenia. It is anticipated that the administration of compositions of the present invention comprising an equivalent or greater number of neutrophil and/or neutrophil precursor cells, either alone or in combination with stem/progenitor cells, should result in the successful reconstitution of a human hematopoietic system in even shorter time. The method of the invention requires, collecting hematopoietic stem and progenitor cells form the patient pre or post therapy, culturing these cells ex vivo with EDHF to generate a population of cells highly enriched in pre-dendritic myelomonocytic progenitor cells, and then administering to the patient a human cell composition enriched for human pre-dendritic myelomonocytic progenitor cells. The cell composition contains at least 50% pre-dendritic myelomonocytic progenitor cells, preferably at least 85% neutrophil precursors. EDHF alone or in combination with other cytokines such as G-CSF, GM-CSF or combinations of these cytokines can be administered to the patient to mobilize stem and progenitor cells prior to the patient treatment for disease. Collected hematopoietic stem and progenitor cells can be cryopreserved and stored for future use. Optionally, the patient may be administered the cytokine G-CSF after infusion of the predendritic myelomonocytic progenitor cell composition in order to promote rapid differentiation into mature neutrophils. The composition may be administered intravenously to a patient requiring a bone marrow transplant in an amount sufficient to reconstitute the patient's hematopoietic and immune systems. The composition may be supplemented with stem cells and other lineage-uncommitted cells.

Once the tri-lineage pre-dendritic myleomonocytic progenitor cells are expanded and/or specific mature myeloid cells are cultured they can be used for engraftment and myelosupportive therapy/support following mycloablation or cytoreductive therapy. Autologous engraftment is preferred. The cultured cells can also be employed in gene therapy and for cancer treatments.

The invention also pertains to kits useful in the methods of the invention. Such a kit contains an appropriate quantity EDHF, and other components useful for the methods. For example, a kit used to facilitate ex vivo expansion of pre-dendritic myelomonocytic progenitor cells contains an appropriate amount of EDHF and ex vivo culture medium.

In one aspect the present invention is a method of expanding, growing, maintaining and/or culturing eukaryotic cells in vitro which comprises culturing eukaryotic cells in the presence of an effective amount of EDHF. The eukaryotic cells include mammalian, insect, plant and invertebrate cells. The cells can be cultured under static or perfusion ex vivo culture conditions. In a preferred embodiment, the eukaryotic cells are human cells. The human cells are selected from the group consisting of skin cells, bone cells, cartilage cells, adipocytes, vessel cells, cells of the oral mucous membrane, urothelial cells, endothelial cells, keratinocytes, mesenchymal stem cells, muscle cells, cells of the nervous-system, hematopoietic cells, tendon cells, hair cells, eye cells, germinal cells, cells of the motility system, embryonic cells, stem cells, liver cells, pancreatic cells, kidney cells, heart muscle cells, epithelial cells, mucous membrane cells, hormone-producing cells and transmitter-producing cells. The cells can be natural cells in culture or genetically modified cells. The cells can be cultured as an autologous transplant or for the preparation of an autologous, allogeneic or xenogeneic transplant such as for example bone marrow transplants. The present invention also includes cell culture systems containing the eukaryotic cells, culture medium and EDHF.

In Vivo Applications

The term “hematopoietic activity” when used herein to describe the effects of EDHF means hematopoiesis as defined above and includes a biological activity elicited by the EDHF, either alone or in combination with other growth factors, which stimulates, proliferates, expands or activates one or more components of the hematopoietic system in a mammal. These biological activities include but are not limited to expansion of hematopoietic stem and progenitor cells, mobilization of blood components from the bone marrow to other organs or into the peripheral blood compartment, expansion of dendritic cells and dendritic cell progenitor/precursor cells, expansion of neutrophils, expansion of Langerhans cells, expansion of monocytes/macrophages, expansion of erythrocytes (red blood cells), expansion of CD11C⁺ cells and expansion of colony forming cells (CFC) including but not limited to CFU-GM, CFU-G, CFU-M, CFU-GEMM, CFU-GM, BFU-E, and CFU-Mk cell types.

The transplantation or engraftment method of the invention described above optionally comprises a preliminary in vivo procedure comprising administering EDHF alone or in sequential or concurrent combination with recruitment growth factors to a patient to mobilize the hematopoietic cells into peripheral blood prior to their harvest. Suitable recruitment/mobilization factors are listed above, and preferred recruitment factors are Flt3 ligand, SCF, IL-1 and IL-3.

Because of its diverse hematopoietic activity and ability to stimulate the proliferation of a number of different cell types and to support the growth and proliferation of hematopoietic progenitor cells, EDHF has potential for therapeutic use in restoring hematopoietic cells to normal amounts in those patients where the number of cells has been reduced due to diseases or to therapeutic treatments such as radiation and/or chemotherapy.

The method of the invention described herein optionally comprises a subsequent in vivo procedure comprising administering EDHF alone or in sequential or concurrent combination with an engraftment growth factor to a patient following transplantation of the cellular preparation to facilitate engraftment and augment proliferation of engrafted hematopoietic progenitor or stem cells from the cellular preparation. An nonexclusive list of suitable engraftment factors, growth factors, colony stimulating factors (CSFs) including; cytokines, lymphokines, interleukins, hematopoietic growth factors, which can be used in concurrent combination or sequential treatment with the EDHF of the present invention include GM-CSF, CSF-1, G-CSF, Meg-CSF, M-CSF, erythropoietin (EPO), IL-1, IL-4, IL-2, IL-5, IL-6, IL-7, IL-8, IL-9, IL-10, IL-11, IL-12, IL-13, LIF, FLT3L/FLK2, human growth hormone, B-cell growth factor, B-cell differentiation factor, eosinophil differentiation factor and stem cell factor (SCF) also known as steel factor or c-kit ligand and variants thereof. GM-CSF concurrent or sequential treatment may also unexpectedly provide an enhanced effect on the activity or an activity different from that expected by the presence of EDHF or the other growth factors.

In vivo treatment with EDHF will stimulate cells of the erythroid lineage thereby improving a patient's hematocrit and hemaglobin levels. EDHF can be administered in this setting alone or in sequential or concurrent combination with cytokines described herein and preferably EPO. Likewise, it's expected that in vivo treatment with EDHF will stimulate megakaryocyte/platelet cell formation thereby improving a patient's thrombocytopenia. The EDHF can be administered alone or in sequential or concurrent combination with cytokines described herein and preferably SCF, IL-3, IL-6, IL-11 and TPO.

The growth factor activity of EDHF is not additive with either optimal concentrations of SCF or Flt3 ligand indicating that a different receptor is involved with EDHF. EDHF is usually more potent than SCF alone, Flt3 ligand alone and optimal concentrations of both cytokines combined.

The in vivo activity of EDHF is of particular interest in treating many disorders of the blood or other disorders that require replacement and/or addition of blood components. The EDHF is administered by any route of administration but preferably by parenteral administration such as, intramuscularly, intravenously or subcutaneously. The subcutaneous route of administration is preferred. The EDHF is administered in amounts of from about 0.1 μg/protein to about 1,000 μg/protein per kilogram of bodyweight. Specific effective dosages can be readily determined by conducting routine dose titration experiments and will vary between specific diseases being treated, clinical presentation and endpoint goals, and each particular patient and their response to the EDHF. Likewise, specific proliferative dosages may be determined for in-vitro and ex-vivo uses.

When EDHF is co-administered with another substance, it may be administered either first or concurrently. The time period between each administration is any time provided that biologically effective amounts of each are present simultaneously at the target cells. Sequential administration involves administering the substances in any order in a subsequent fashion.

In another embodiment of the present invention EDHF may be used to speed the healing of wounds resulting from trauma, disease or surgery. Stem cells are found throughout many different tissues and have the potential to differentiate to whatever tissue cells are needed to allow for tissue repair. Endothelial cells are found wherever blood is delivered and need to replace those lost as a result of the wound. Given that EDHF has been shown to stimulate proliferation of stem cells as well as endothelial cells, it is an embodiment of the present invention to administer EDHF to a patient in need of wound repair. Such administration may be done locally at the site of tissue damage, to a fluid contacting the site of tissue damage (e.g. snovial for cartilege damage) or systemically. Given that wounds may become infected, the added immunostimulatory effect would likewise be beneficial to healing.

In another embodiment of the present invention a mammal undergoing myeloablation therapy is treated by: (a) obtaining peripheral blood from the mammal wherein the blood following stem cell mobilization treatment using EDHF or EDHF in combination with other known mobilization cytokines such as G-CSF and/or GM-CSF is enriched for hematopoietic stem and progenitor cells (b) isolating hematopoietic progenitor cells (c) culturing the hematopoietic stem and progenitor cells in the presence of an effective amount of EDHF to preserve and enrich the hematopoietic stem and progenitor cells; and (d) administering the cultured cells to the mammal following the myeloablation to reconstitute the hematopoietic system of the mammal. The myeloablation therapy can be bone marrow irradiation, whole body irradiation, chemically induced myeloablation or combinations thereof. The culturing step (c) can be conducted in the presence of additional growth factors if desired. A nonexclusive list of suitable factors growth factors, colony stimulating factors (CSFs) including; cytokines, lymphokines, interleukins, hematopoietic growth factors, which can be used in concurrent or sequential treatment with the EDHF of the present invention includes GM-CSF, CSF-1, G-CSF, Meg-CSF, M-CSF, erythropoietin (EPO), IL-1, IL-4, IL-2, IL-5, IL-6, IL-7, IL-8, IL-9, IL-10, IL-11, IL-12, IL-13, LIF, FLT3L/FLK2, human growth hormone, B-cell growth factor, B-cell differentiation factor, eosinophil differentiation factor and stem cell factor (SCF) also known as steel factor or c-kit ligand and variants thereof.

The term “autologous transplantation or engraftment” is described in U.S. Pat. No. 5,199,942, which is incorporated herein by reference. Briefly, the term is consistent with the definition of autologous stem cell infusion as defined above and includes a method for conducting autologous hematopoietic progenitor or stem cell transplantation, comprising: (1) collecting hematopoietic progenitor cells or CD34⁺ stem cells from a patient prior to cytoreductive therapy; (2) expanding the hematopoietic progenitor cells or stem cells ex vivo with EDHF to provide a cellular preparation comprising increased numbers of hematopoietic progenitor cells or stem cells; and (3) administering the cellular preparation to the patient in conjunction with or following cytoreductive therapy. Progenitor and stem cells may be obtained from peripheral blood harvest, cord blood, cytokine mobilized stem cells or bone marrow explants. Optionally, one or more growth factors can be combined with EDHF to aid in the proliferation of particular hematopoietic cell types or affect the cellular function of the resulting proliferated hematopoietic cell population. Of the foregoing, Flt3 ligand, SCF, IL-1, IL-3, EPO, G-CSCF, GM-CSF and GM-CSF/IL-3 fusions are preferred, with G-CSCF, GM-CSF and GM-CSF/IL-3 fusions being especially preferred. The term “allogeneic transplantation or engraftment” means a method in which bone marrow or peripheral blood progenitor cells or stem cells are removed from a mammal and administered to a different mammal of the same species. The term “syngeneic transplantation or engraftment” means the bone marrow transplantation between genetically identical mammals.

In another embodiment of the present invention cells produced by the present invention are useful for genetic therapy. In this embodiment, EDHF is administered to cells in vitro to stimulate cell cycling and incorporation of a foreign gene. Stem cells represent the ideal target for genetic therapy because they are self renewing and may differentiate many different cell types. However, stem cells cycle at such a low rate that incorporating any foreign genes for gene therapy is difficult. Since EDHF has been shown to stimulate stem cell growth, and especially growth of pre-dendritic myelomonocytic progenitor cells, EDHF represents a good treatment for cells being transfected with heterologous genes useful in gene therapy. The same treatment may be applied for cells being transfected for non-gene therapy purposes.

Also, EDHF enhances cell proliferation which is desirable in order to have a larger number of transformed cells before infusion. EDHF administration may be continued after infusion to encourage expansion in situ. This would be particularly useful for treating diseases of secreted protein deficiency (either absent or defective such as ADA, insulin, HGH, lysosomal storage enzymes, tumor suppressors, etc.). This may also be used to provide protein drugs to the patient continuously such as anti-cancer factors/antibodies and other lifelong term administration of drugs. This may also be used to produce “normal” cells to replace defective ones such as in thalasemias and sickle cell anemia. Vectors for DNA repair/mutation/inactivation and antisense vectors may also be used for situations where abnormal expression of an endogenous gene is undesired. The physical steps involve removing/obtaining the cells, culturing them in vitro with EDHF and transforming them with a vector containing the therapeutic gene/oligonucleotide, optionally further culturing in-vitro with optional selection, isolation or expansion of transformed cells and reintroducing them into the person. Recombinant viral vectors are particularly preferred.

In addition to the transplantation of hematopoietic stem cells, the transplantation of other tissues would also benefit from co-administration of EDHF and/or continuing treatment during healing after transplantation. Whether autologous or heterologous transplantation, the stem cells, particularly those not involved in immunity, would aid in the incorporation of transplanted tissues. In reconstructive surgery, cosmetic surgery, heart bypass surgery, skin grafts, orthopedic surgery and the like, tissues from one part of the body are transplanted to another part. In both the donor region and the receiving region a scarcity of tissue remains or is added. Activation of endogenous stem cells at both sites to effect replacement of tissue is desirable. EDHF is administered before, at the time of transplantation and during recovery to enhance acceptance of the graft and speed replacement of tissue.

In the situation of transplantation between different individuals, the same situation applies to both donor and recepient who both benefit from the administration of EDHF. During allotransplantation, hematopoietic stem cells are not transferred to avoid graft-vs-host disease. While such treatment is preferred for tissue transplants, whole organ transplants may also be enhanced by the administration of EDHF.

Vaccine Enhancement/Vaccine Adjuvant

The present invention also includes a method of enhancing the immune response in a mammal receiving a vaccine, which comprises administering an effective immune enhancing amount of EDHF in conjunction with the administration of the vaccine. The EDHF can be co-administered with the vaccine simultaneously or on the same day or administered up to a few days after the vaccine, but it is preferably used as a pretreatment 1-14 or more days prior to the administration of the vaccine. The EDHF is preferably administered intramuscularly or subcutaneously at dosages that are described above for increasing hematopoiesis. The EDHF stimulates/activates and expands (increases the number of cells) the dendritic precursor cells and mature dendritic cells which are the antigen presenting cells of the immune system. The increase in dendritic cell function allows the mammal, preferably a human, to mount a better immune response to the vaccine compared to if the mammal received the vaccine alone. The EDHF can also be administered along with another growth factor as described herein. Additionally, it is preferred that the vaccine is administered with a vaccine adjuvant/immunostimulatory molecule such as CD40L, LSP, or CpG DNA and variants thereof.

The present use of EDHF in conjunction with vaccines increases the efficacy and potency of the vaccine. The advantage of this utility of EDHF is that the currently available vaccines can be improved without biological modification of the vaccine. Vaccines contain one or more antigenic determinants to illicit an immune response by the mammal in order to provide immunity to the mammal from one or more pathogens possessing the antigenic determinant(s). The use of the EDHF with vaccines represents an improvement whereby the antigenic response of the mammal being treated with EDHF is enhanced or improved compared to the vaccine being administered alone and may result in fewer or less frequent booster immunizations. EDHF may also be used with a second or subsequent dosage of a vaccine.

The EDHF can also be used to promote or enhance immune response in mammals without co-administration of a vaccine. The EDHF, given in doses described herein, will increase the number and activate dendritic cells and dendritic progenitor cells whereby the body can respond better to invading antigens. Included in the dendritic cells that are activated and expanded are Langerhans cells and CD11c⁺ MHCII⁺ CD86⁺ wherein the elevated CD11c⁺ cells have been observed in the spleen.

The following examples illustrate the practice of the present invention and should not be construed as limiting its scope.

### EXAMPLE 1

### Hematopoietic Stem Cell Culture Medium Conditioned with Soluble Proteins >30 kDa Derived from Porcine Microvascular Endothelial Cells

The following procedures were employed to prepare a fraction of porcine brain endothelial cell derived proteins having a MW>30 kDa (EDHF). The EDHF was used to stimulate or accelerate the proliferation of hematopoietic stem cells and progenitor cells obtained from human cord blood. The resulting expanded hematopoietic stem cells and progenitor cells were then induced to differentiate along the granulocyte, monocyte and dendritic cell (DC) lineages when cultured with the appropriate cytokine combinations.

A. Porcine microvascular endothelial cells: Primary porcine brain microvascular endothelial cells (passages 26-35) were maintained in endothelial cell culture medium consisting of M199 medium (GIBCO LIFE Technologies, Gaithersburg, Md.) supplemented with 10% FBS (Hyclone, Logan Utah), 30 μg/mL endothelial cell growth factor supplement (Sigma, St. Loius, Mo.), 100 μg/ml L-glutamine, 100 U/ml penicillin/streptomycin solution, and 50 μg/mL preservative-free sodium heparin (Sigma, St. Louis Mo.)) and passaged weekly at 1×10⁶ cells per gelatin-coated 75 cm² flask.

B. Production of porcine derived hematopoietic factor (EDHF): For the production of EDHF, porcine microvascular endothelial cells (EDHF) were grown to confluency in complete endothelial cell culture medium consisting of M199 supplemented with 10% heat-inactivated FCS, 50 μg/ml preservative-free heparin, 100 μg/ml L-glutamine, and 100 U/mL penicillin/streptomycin solution. Once the endothelial cultures were 90-100% confluent, the endothelial cell monolayers were washed twice with PBS, and refed Iscove's (IMDM) medium without serum. After an additional 7 days of culture, the culture medium was harvested, filtered through a 0.2 μm membrane to remove cell debris, and proteins >30 kDa were concentrated 10-70× by ultra filtration using an YM-30 Amicon membrane. The concentrated EDHF was passed through a 0.2 μm filter, aliquoted, and stored at −20° C. to −80° C. All batches of EDHF were tested for their ability to support human CD34⁺ hematopoietic cell proliferation.

C. Isolation of CD34⁺ hematopoietic progenitor cells: Human cord blood (CB) was obtained during normal full-term deliveries after informed consent was given. CB samples (50-150 mL) were diluted 1:4 with Dulbecco phosphate-buffered saline (DPBS) Ca⁺⁺- and Mg⁺⁺-free (GIBCO-BRL, Grand Island, N.Y.). Diluted CB was then underlaid with Ficoll-Paque (Pharmacia AB, Uppsala, Sweden), and centrifuged at 800 g for 30 minutes at 20° C. The mononuclear cell fraction was collected and the CD34⁺ cells were immunomagnetically enriched using the MACS CD34 Isolation Kit (Miltenyi Biotec, Auburn, Calif.). Procedures were performed as per manufacturer's recommendations. Cells were incubated with hapten-labeled anti-CD34 antibody (QBEND-10, Becton Dickinson) in the presence of blocking reagent, human IgG (Bayer, Elkhart, Ind.), and then with antihapten coupled to MACS microbeads. Labeled cells were filtered through a 70 μm nylon mesh and separated using a high-gradient magnetic separation column. Magnetically retained CD34⁺ cells were eluted following several washes of the column with D-PBS. The purity of the CD34⁺ population was routinely more than 90%. CD34⁺ cells were either used for experimentation or cryopreserved in 10% dimethylsulfoxide (Sigma), 50% fetal calf serum (FBS, Hyclone Laboratories, Logan, Utah) by controlled-rate freezing methods. Following thawing, samples were usually pooled to provide sufficient cell numbers for each experiment.

D. Cytokines: Recombinant human stem cell factor (SCF), thrombopoietin (TPO), FLT3 ligand (FLT3L), granulocytes-macrophage colony-stimulating factor (GM-CSF), interleukin-4, and tumor necrosis factor α (TNF-α) were purchased from Peprotech (Rocky Hill, N.J.). All cytokines were pure recombinant molecules and were used at concentrations that induced an optimal response in cultures of human CB CD34⁺ cells. The concentrations used were 50 ng/mL for SCF, 50 ng/mL FLT3L, and 20 ng/mL for TPO, GM-CSF, IL-3, IL-4 and TNF-α.

E. Ex vivo expansion cultures: To promote differentiation of purified human CB CD34⁺ cells, a stroma-free suspension culture was established as previously described. Tissue culture dishes (35 mm; Coming, Corning, N.Y.) were seeded with 2×10⁵ CD34⁺ cells/well in 3 mL RPMI 1640 medium (GIBCO-BRL, Grand Island, N.Y.) containing 2 mmol/L L-glutamine, 10 mmol/L HEPES, 50 IU/mL penicillin, 125 μg/mL streptomycin, 10% fetal bovine serum (FBS; Hyclone, Logan, Utah) and 50 mM ME. Cultures were placed at 37° C. in 100% humidified atmosphere of 5% CO₂ in air. At initiation of cultures and at 7 day intervals, cultures were treated with a previously determined optimal concentration of EDHF (1:70 dilution, 1×final concentration of the 70×stock >30 kD MW) in the presence or absence of an optimal combination of recombinant cytokines: stem cell factor (50 ng/mL), FLT3L (50 ng/mL), and TPO (20 ng/mL) (R&D Systems, Minneapolis, Minn.). Cultures were maintained at a cell concentration of 5×10⁵ to 2×10⁶ viable cells/mL. Cultures were maintained for 5 weeks with medium replenished every 7 days Cells were harvested from each culture condition at selected times and assayed for total viable cell yield by trypan blue dye exclusion (Sigma, St. Louis, Mo.).

F. In vitro differentiation of ex vivo expanded dendritic cell (DC) precursors: Nonadherent cell populations of cells generated in the above-described primary cultures of CD34⁺ cells for 7-28 days were harvested, resuspended to obtain single-cell suspensions, and plated in secondary 3 mL cultures at 1×10⁶ cells/mL. Stimulation of DC development in these secondary cultures was accomplished by the addition of GM-CSF (20 ng/mL), IL-4 (20 ng/mL) plus TNF-α (20 ng/mL) for the final 4-7 days of culture.

G. Cell surface phenotyping and microscopic analysis: For flow cytometeric staining, cells harvested from cultures were suspended in phosphate-buffered saline (PBS) with 0.2% deionized fraction V bovine serum albumin (BSA) (GIBCO, Grand Island, N.Y.) at a concentration of 5×10⁵ cells per tube. Phenotypic analysis was performed by immunofluorescence flow cytometry using a panel of saturating concentrations of either directly conjugated FITC conjugated anti-CD14 (mIgG2b, clone MoP9), PerCP-conjugated CD34 (IgG1, clone 8G12), PE conjugated CD38 (IgG1, clone Leu-17), PE-conjugated CD33 (IgG1), PE-conjugated anti-CD1a (mIgG1, clone BL6), PE-conjugated anti-CD83 (mIgG2b, clone HB15a), PE-conjugated anti-CD86 (mIgG2b, clone IT2.2), and FITC conjugated HLA-DR (mIgG2a, clone L243) which were all purchased from BD-Pharmingen (San Diego, Calif.). Appropriate conjugated isotype-matched antibodies were used as controls. In all experiments, cell samples were preincubated in 0.1 mL PBS supplemented with of blocking reagent, human IgG (Bayer, Elkhart, Ind.) for 30 minutes to block nonspecific binding. After a wash with PBS, cells were stained for 30 minutes on ice with various monoclonal antibodies (mAbs) conjugated by fluorescein isothiocyanate (FITC) or phycoerythrin (PE). Stained cells were washed with PBS, fixed with 0.5 mL of 1.6% paraformaldehyde (Electron Microscopy Sciences, Fort Washington, Pa.). Cells were analyzed on a FACScan flow cytometer (Becton Dickinson) calibrated with Calbrite beads (Becton Dickinson) for FITC and PE. The distribution of debris, dead cells, and any contaminating red blood cells was assessed on the basis of forward and right-angle scatter before proceeding with the analysis. A total of 10,000 events were examined using a 488-nm wavelength excitation. Acquired events were analyzed using Cell Quest Software (Becton Dickinson). Results were expressed as percent positive cells after subtracting negative control values.

Wright-Giemsa staining was performed on cytocentrifuge cell preparations of freshly isolated and cultured cord blood CD34⁺ cells. Cells were suspended in 40% FBS at 1×10⁵ cells/mL. One hundred microliters of cell suspension was spun onto glass microscope slides. Slides were air dried, cells methanol fixed, and stained with Wrights-Giemsa stain. Cells were visualized and representative cells were photographed using phase 50×high dry hematology lens (×500 magnification, Olympus Optics, Mellville, N.Y.).

H. Mixed leukocyte reaction (MLR) Untouched human T cells were purified from peripheral blood MNC (PBMNC) by negative immunomagnetic depletion using the MACS separation procedure in accordance with the direction provided by the manufacture (Miltenyi Biotec, Auburn, Calif.) as previously described. Peripheral human blood was collected in preservative-free heparinized syringes. Peripheral blood mononuclear cells (PBMC) were separated by centrifugation on Ficoll-Hypaque (Sigma, St. Louis, Mo.). To obtain highly purified CD4⁺ T cells, the MNCs were depleted of non-CD4⁺ T cells using a cocktail of mouse antihuman mAbs (anti-CD8, CD14, CD15, CD19, CD56) followed by immunomagnetic depletion using goat anti-mouse Ab conjugated superparamagnetic microbeads (Miltenyi Biotec, Auburn, Calif.), thereby isolating untouched (no Ab bound) CD4⁺ T cells by magnetic cell sorting. Graded numbers of mitomycin-C treated (MMC; 15 μg/mL, Sigma St. Louis, Mo.) stimulator cells (generated DC) were added to constant numbers (5×10⁴/well) of purified (greater than 98%) allogeneic CD4⁺ T cells in round-bottom 96-well tissue culture plates (Costar). Cells were cultured in RPMI 1640 (GIBCO) supplemented with 5% heat-inactivated autologous serum and triplicate analyses were performed on each sample preparation. After 7 days of culture, stimulation of responding CD4⁺ T cells was determined using the AlamarBlue assay a colorimetric growth indicator based on the detection of metabolic activity. 20 μL of AlarmarBlue (5 mg/mL in PBS) was added into each well and the plates were incubated at 37° C. for an additional 6 hours. The resultant absorbance at 570 nm was read by a microplate immunoreader.

I. CFU-GM assay. Day 21 EDHF expanded cells or DC precursors generated in culture, were plated in semi-solid methylcellulose medium containing optimal amounts of IL-3, SCF, and GM-CSF (Methocult GF H4534; Stem Cell Technologies Inc, Vancouver, BC, Canada) at a concentration of 1-5×10⁴ cells/culture dish, and incubated for 14 days. CFU-GM colonies (>50 cells) were counted by visual examination of the plates, according to standard methods. The mean colony count per 10⁵ cells was calculated.

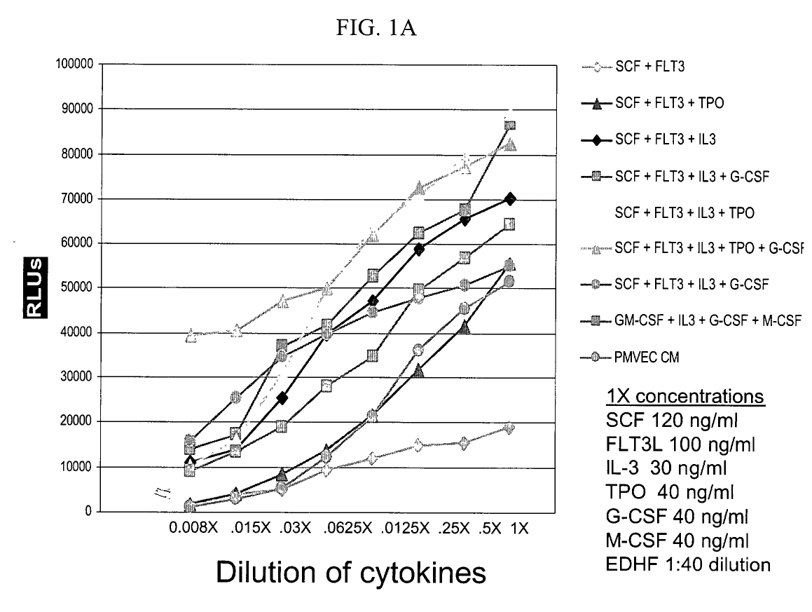

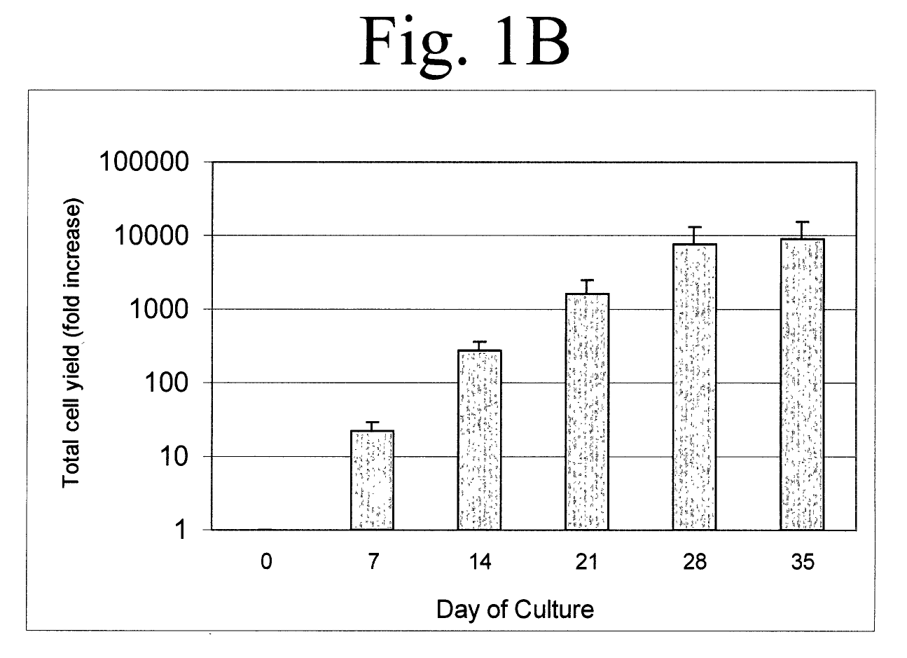

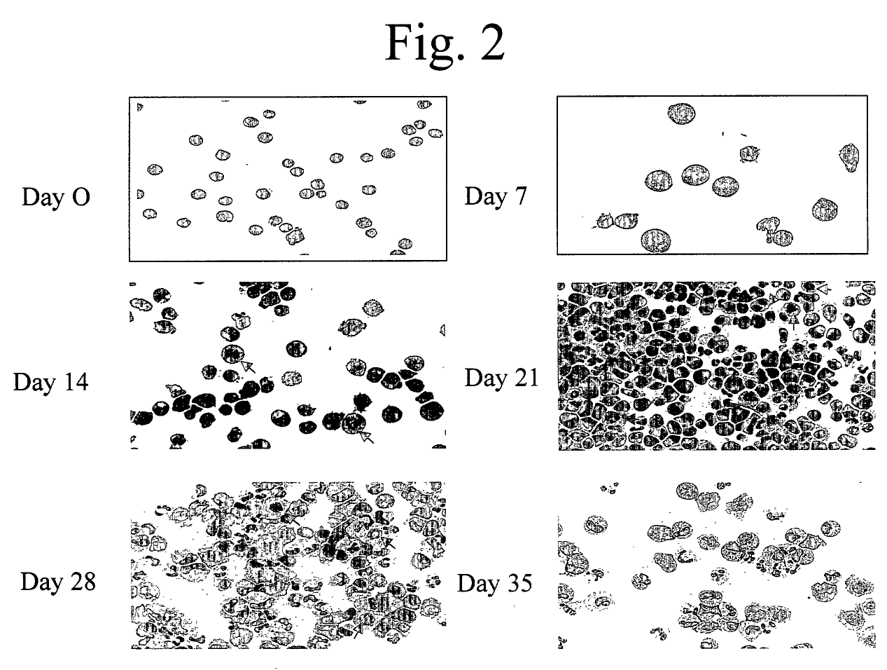

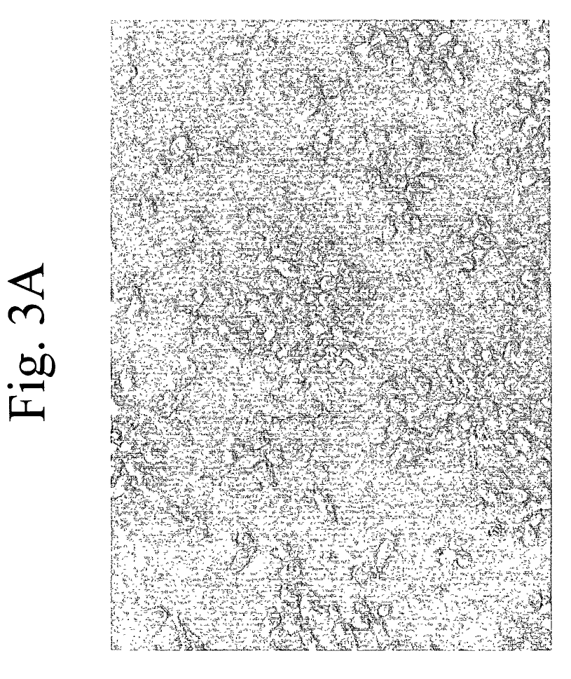

J. Proliferative effects of EDHF on cord blood CD34⁺ cells. Results from a representative experiment shown in FIG. 1A demonstrate the potent effects of EDHF on cord blood CD34⁺ cell proliferation at various concentrations in comparison to various concentrations of other known hematopoietic growth factors. Initial studies were designed to examine if EDHF as a single stimulus could sustain long-term expansion of cord blood CD34⁺ progenitor cells and as well as lineage specific precursors in vivo. First, the proliferative effects of EDHF on freshly-isolated immunomagnetic selected CD34⁺ cord blood cells, the purity which was ranged between 90-98% CD34⁺ cells, was examined. In 8 separate experiments, freshly isolated CD34⁺ cells, isolated from 8 different cord blood samples, were cultured with an optimal concentration of EDHF, which was determined in preliminary experiments to induce maximal cellular CD34⁺ cell proliferation and progenitor cell production. As shown in FIG. 1B, after 7, 14, 21, 28 and 35 days of culture, the total number of cells in culture increased approximately 22, 281, 1631, 7580, and 8949-fold, respectively. Note the y-axis is on a logarithmic scale. In contrast, treatment with SCF, FLT3L plus TPO was far inferior with 10-, 43-, 97-, 121- and 143-fold increases in total cell number measured at the same time intervals. At these specific time intervals, aliquots of proliferating cells were harvested, counted and cytocentrifuge cell preparations made and then stained with Wright-Giemsa (FIG. 2). It was observed that the majority of the cells up to 14 days of culture had immature blast cell morphology and mitotic figures. Few mature cells (neutrophil and macrophages) and late precursor cells were evident during this early culture period. Cells from day 21-35 suggest a high degree of committed myeloid differentiation toward the neutrophil and macrophages lineages as evident by the appearance of myeloid blast cells, promyelocytes, myelocytes, metamyelocytes, and monoblasts. Numerous mature neutrophils and macrophages were abundant in culture by day 28 and 35 of culture.

Prior to 21 days of culture, no monocyte or granulocyte-related cells were detected in culture as indicated by the absence of CD14⁺ and CD86⁺ cells; these markers were also not detected at earlier times of culture (days 2-6), suggesting that there was not even transient expression of early granulocyte markers. Also, evident was the lack of CD1a and CD83, which are typical DC lineage surface markers.

Using day 21 generated cells, CFU-GM assays were performed to determine their myeloid colony-forming potential. CFU-GM colony-forming cells responsive to SCF+GM-CSF+IL-3 were largely abundant (1734±435 CFU-GM per 1×10⁵ plated cells) following 21 days of pre-culture in the presence of EDHF alone. In contrast, when the same day-14 generated cells were cultured for the last 7 days with the additional supplementation of GM-CSF+IL-4+TNF-α neither CFU-GM nor CFU-DC colonies were observed; rather, only single viable cells were observed across the plates.

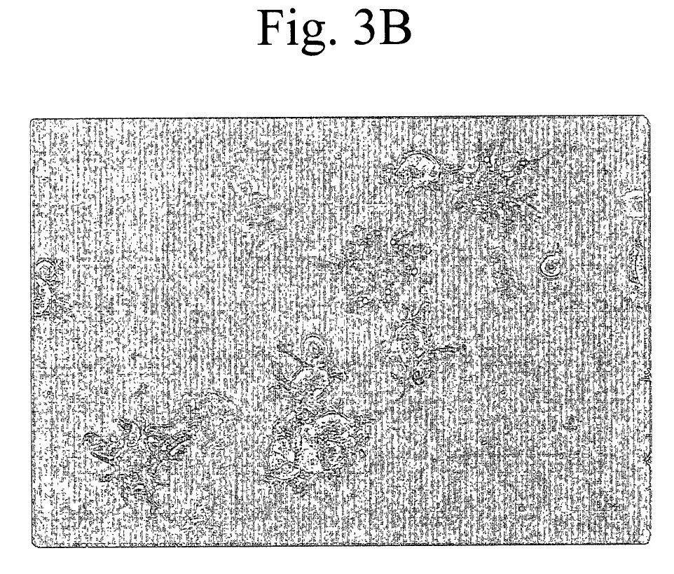

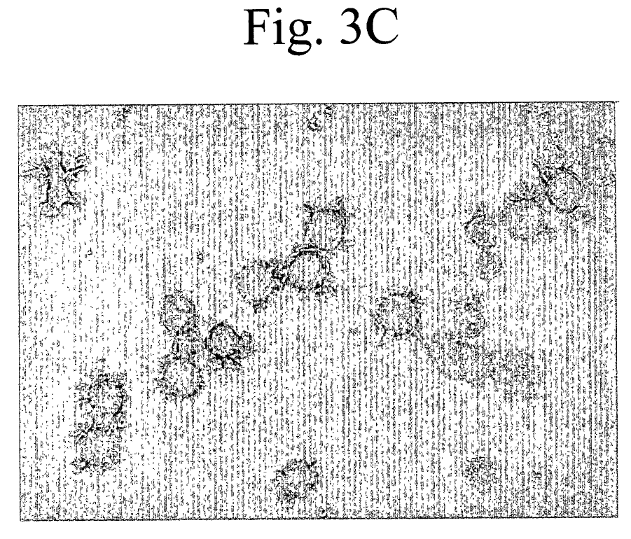

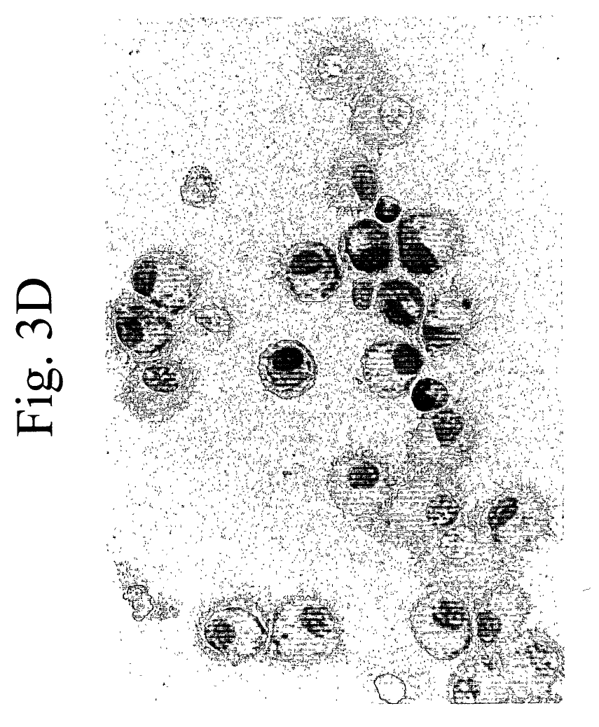

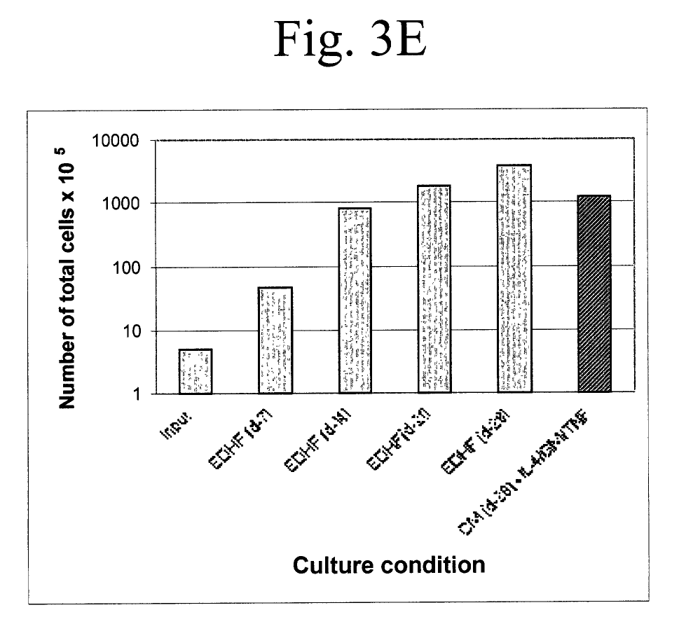

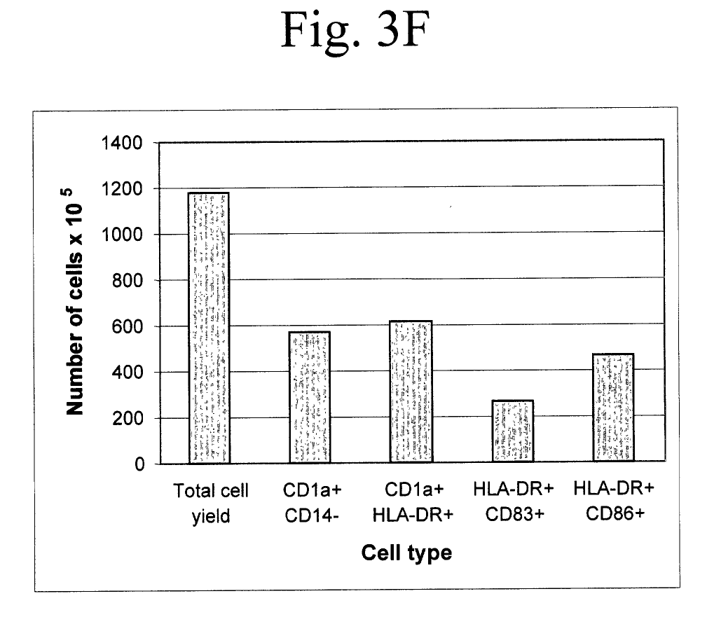

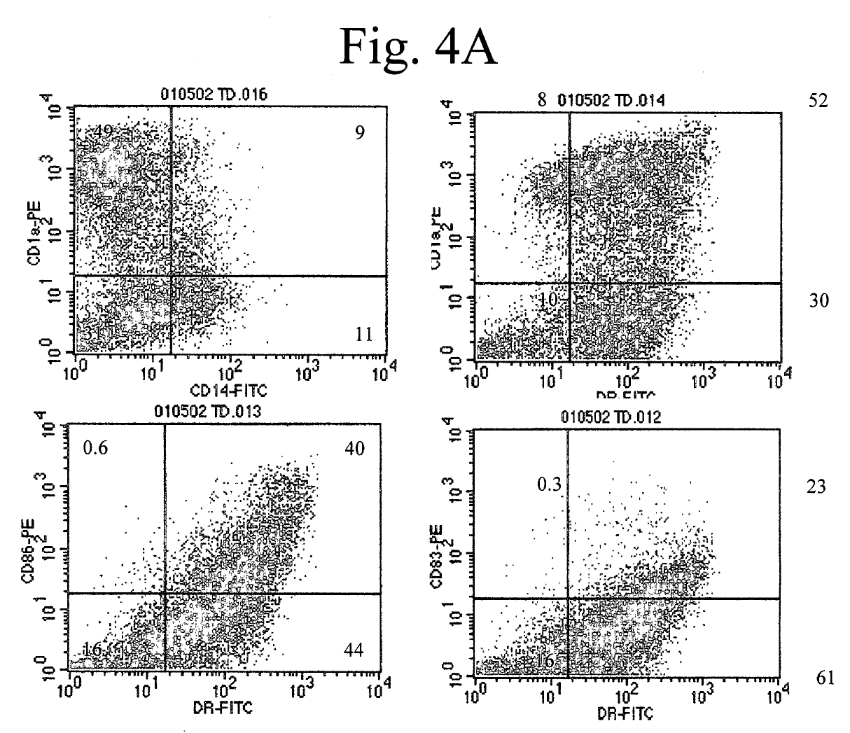

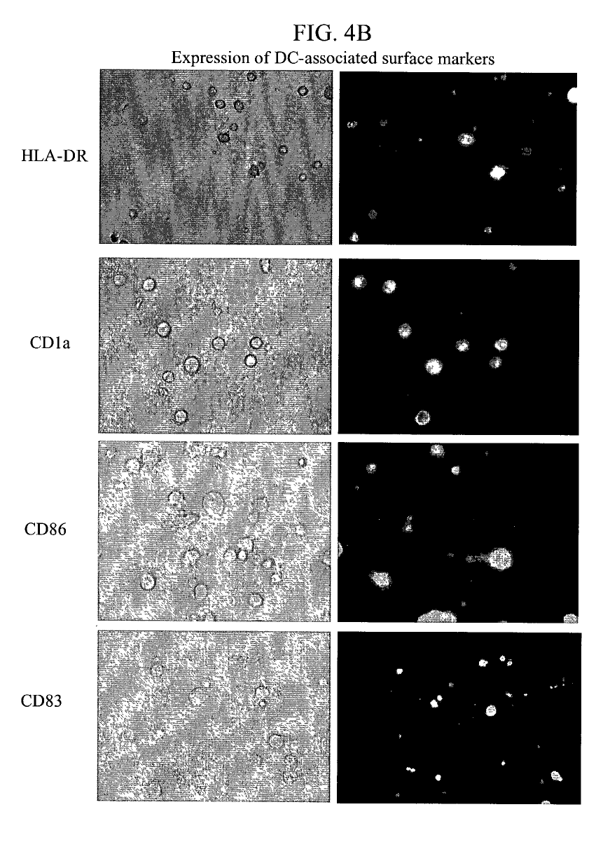

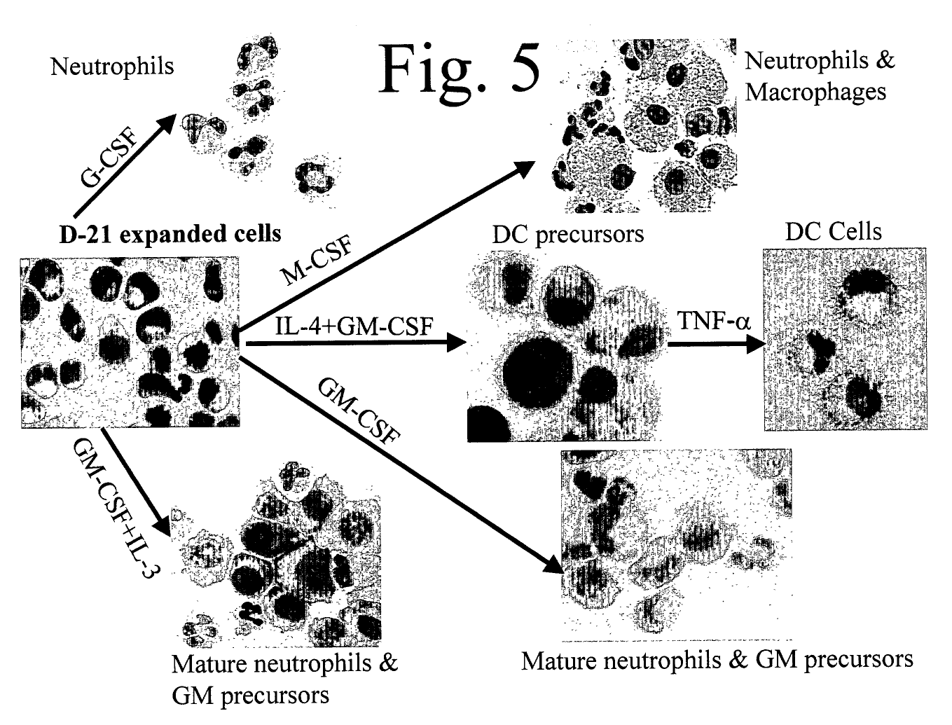

K. EDHF supports the expansion of hematopoietic progenitor cells capable of multilineage differentiation. A more detailed analysis of the cells derived from cord blood CD34⁺ cells treated with EDHF alone for 14 days was performed. Specifically, it was investigated whether the homogenous populations of presumptive DC and myeloid progenitor/precursors generated between days 14 and 21 of culture could be triggered to differentiate into mature lineage specific cells using GM-CSF+IL-4+TNF-α for stimulating DC development, M-CSF for macrophage specific differentiation, G-CSF for neutrophil specific differentiation, GM-CSF for mixed macrophage and neutrophil differentiation, and TPO+IL-3 for megakaryocyte differentiation. As shown in FIG. 3, EDHF treated cells from day 21 cultures were treated with the cytokine cocktail of GM-CSF, IL-4 and TNF-α resulting in the conversion from immature DC precursors cells to mature, loosely adherent multi-cellular aggregates and cell clusters appeared in culture, which increased in size and number with time (FIGS. 3A and 3B). These features were highly reminiscent of the DCs produced by human monocytes or mouse bone marrow in which proliferating and differentiating DCs accumulate as immature DCs in analogous cell clusters. After an additional 3-4 days of culture in induction medium the cells were totally nonadherent, more dispersed and showed multiple long processes characteristic of mature DC (FIG. 3B&C). As shown in FIG. 3D, day 14 EDHF generated cells, when cultured in the presence of GM-CSF+IL-4+TNF-α, gave rise to cellular progeny that were homogeneously large and contained numerous dendrite projections and a polar oriented nucleus. The combined DC induction signal of GM-CSF+IL-4+TNF-α appeared to have anti-proliferative effect on further EDHF-induced stimulation (FIG. 3E). These ex vivo generated DC-like cells expressed high levels of CD1a and HLA-Dr surface markers (FIG. 3F and FIG. 4) after 4-5 days of DC induction whereas a larger percentage of the total cells exhibited the typical mature DC phenotype CD1a⁺CD14⁻CD83⁺CD86⁺ (FIG. 4B) after 7-10 days of DC induction. Importantly, and in contrast to what has been reported for cord blood, CD34⁺-derived DC using other cytokine cocktails for DC precursor growth and DC maturation, no CD14⁺ intermediate were observed in our culture from days 7-14. In striking contrast, when the same precursors were further propagated for an additional 7 days in the presence of EDHF, cells emerged that hardly displayed the DC and/or mature myeloid-associated features. Interestingly, when EDHF-derived cells collected at day 21 of culture were cultured in the presence of 20 ng/mL M-CSF, but not in its absence, a large percentage of the cells matured into mature macrophages (FIG. 5). By light microscopy, these generated cells from M-CSF treated cultures displayed a macrophage morphology, a strong adherence to plastic surface, small indented nuclei, foamy cytoplasms with numerous intracytoplasmic vacuoles, and no evidence of cytoplasmic dendritic projections. Moreover, it was observed that EDHF-derived day 21 precursor cells can also develop into mature segmented neutrophils in the presence of G-CSF whereas GM-CSF and/or IL-3 stimulation supports the production of both a mixed population of cells consisting of neutrophils and macrophages. In addition, we have some preliminary results that suggest that controlled maturation of day 14-21 EDHF generated DC precursors can also be accomplished by using proinflammatory agents such as LPS (data not shown), and CD40L ligation is also being considered.

Overall, these findings show that day 14 EDHF generated precursors from cord blood CD34⁺ cells follow the myeloid/DC differentiation pathway to the branching point of the DC, granulocytic and monocytic lineages.

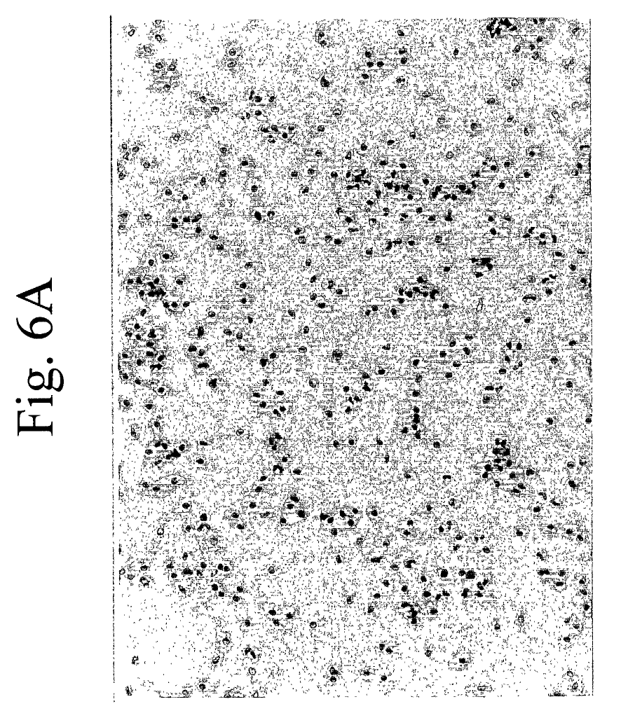

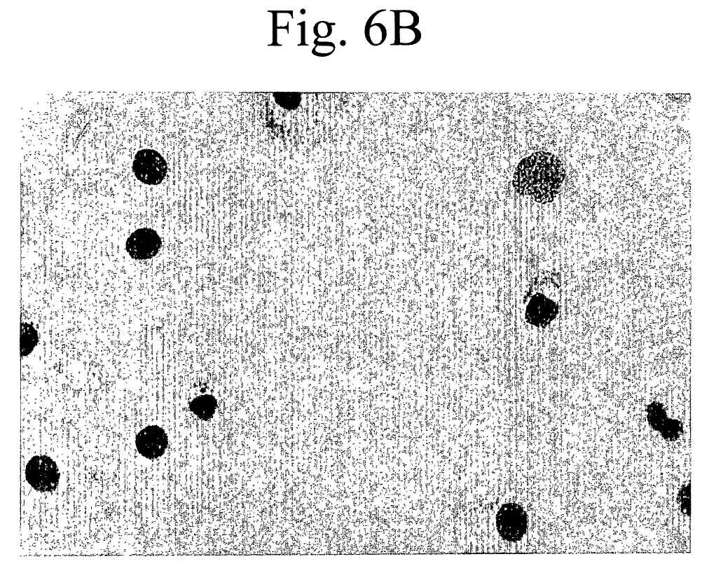

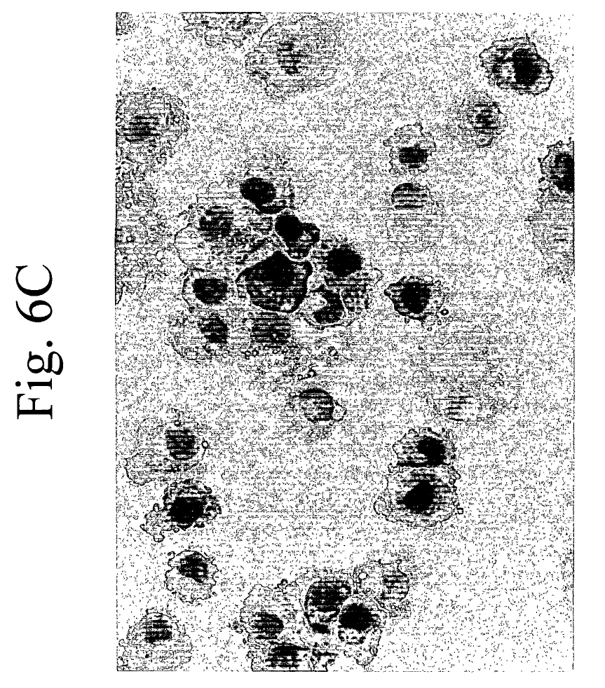

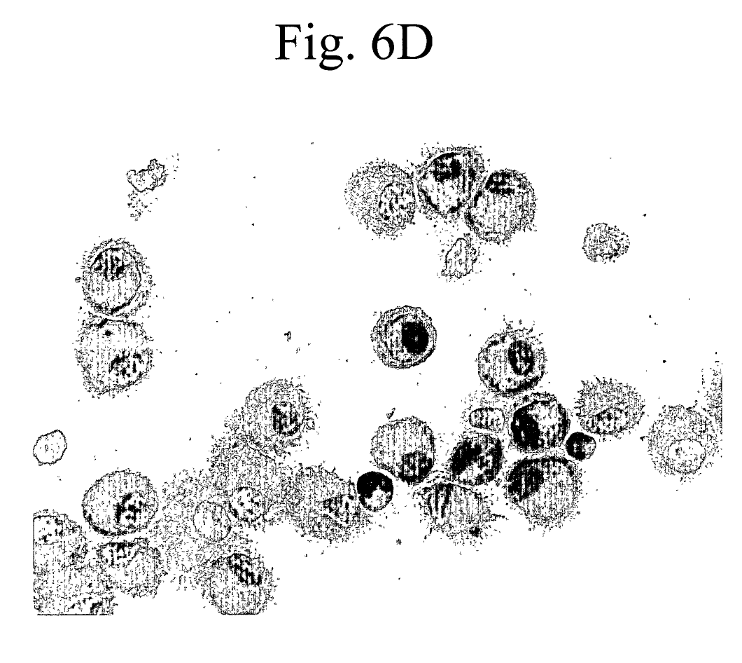

L. Multiple stages of DC differentiation from EDHF derived DC precursors. In culture, it was frequently observed that there were three distinct stages of DC differentiation using day 14-21 EDHF generated cells that had been treated with GM-CSF+IL-4+TNF-α for an additional 7 days. In the early phase of the induction treatment, (3-5 days), a modest population of large, adherent, Langerhans-like cells developed (FIG. 6A&B) while the remaining cells exhibit the typical activated DC morphology (FIG. 6C). Intermediate HLA-DR expression, high expression of CD1a, and the lack of the costimulatory molecule CD80 as well as DC-associated molecule CD83 characterized these adherent LC-like cells. During the next 3-7 days of culture (later phase) in the presence of the same DC induction cytokine cocktail, the LC-like population lost plastic adherence and matured into free-floating nonadherent round cells exhibiting a corona of thin dendrites. In addition to the morphological changes, these cells expressed increased levels of surface HLA-DR, costimulatory molecule CD86, and DC lineage-associated Ag CD83. More or less these immature DC took another 4-5 days to acquire the morphology and phenotypic characteristics typical of activated mature DC (FIG. 6D).

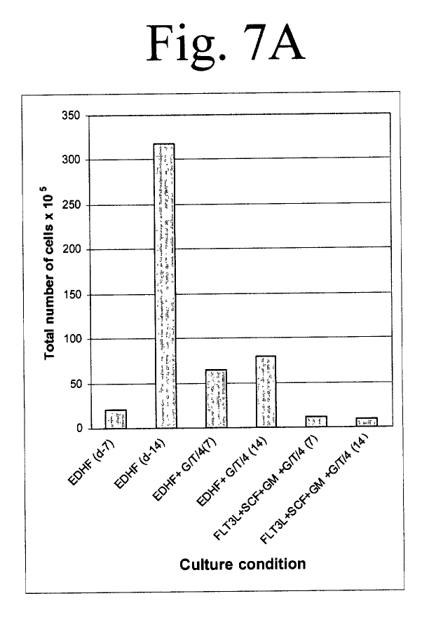

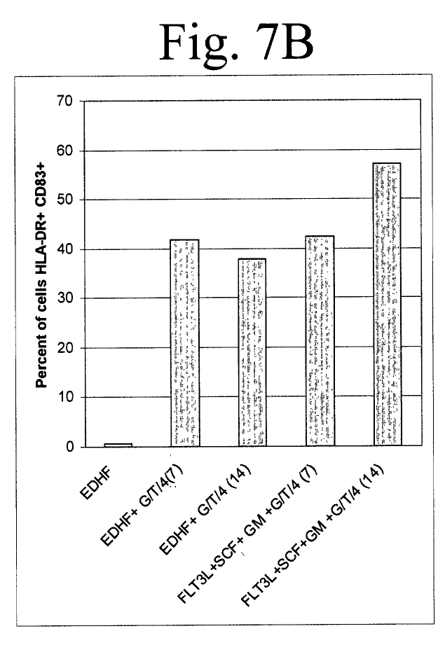

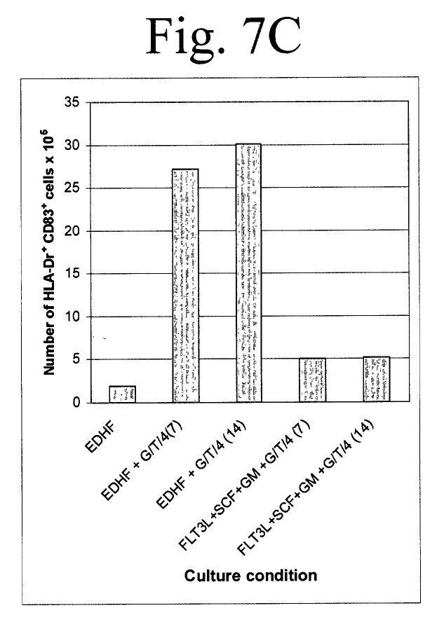

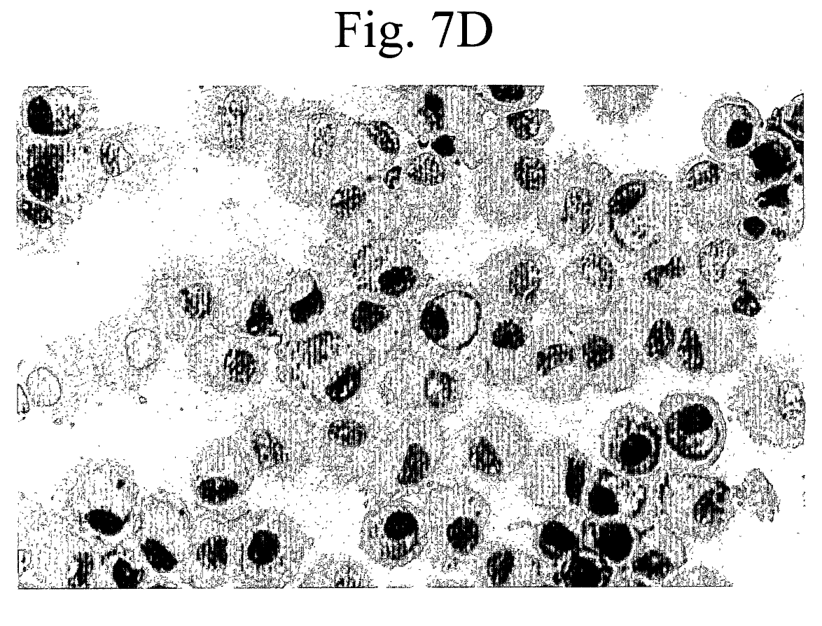

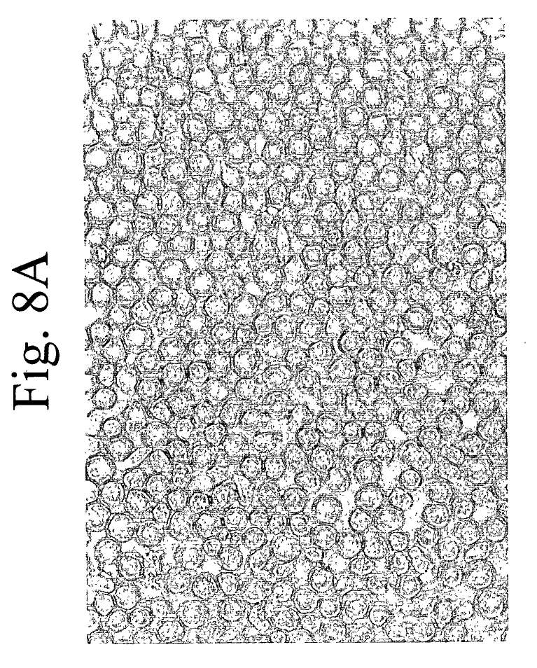

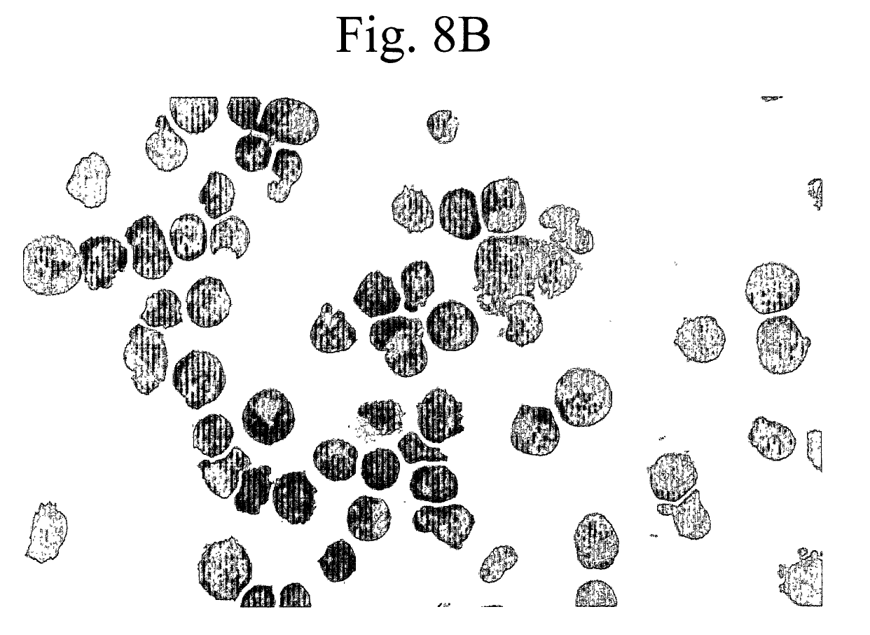

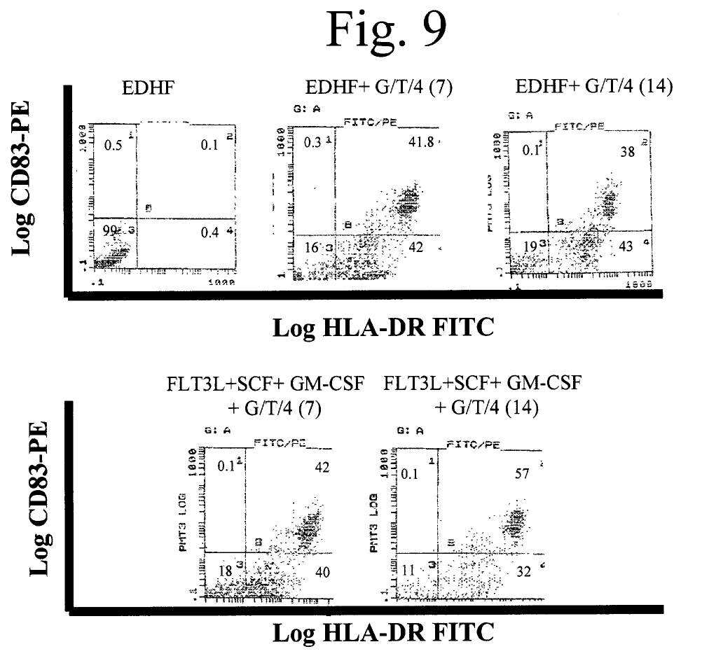

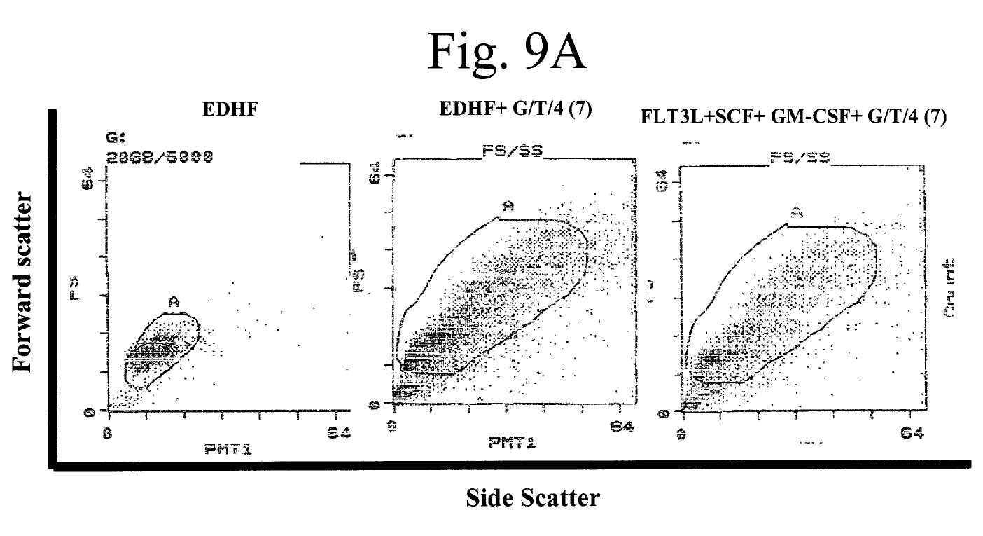

M. Comparison of EDHF versus the cytokine combination of SCF+FLT3L+GM-CSF used for the ex vivo expansion of DC precursors from cord blood CD34⁺ cells. Before analyzing the functional activity of EDHF-derived DC, a comparison was made between the efficacy of DC precursor expansion in EDHF versus SCF+FLT3L+GM-CSF treated cultures. Fresh highly purified cord blood CD34⁺ cells were cultured with either EDHF or in the presence of FLT3L+SCF+GM-CSF for 14 days with GM-CSF+IL-4+TNF-α added for the entire culture period or for the final 7 days of incubation. After 14 days of culture, all cells were collected from each culture condition, washed and assayed for total viable cells using trypan dye exclusion (FIG. 7A). Total cell number expansion was greatest (˜124-fold) in EDHF treated cultures without the addition of the DC induction cytokines GM-CSF+IL-4+TNF-α. Cells from these cultures demonstrated no evidence of DC-like morphology (FIGS. 8A and 8B). In comparison, GM-CSF+IL-4+TNF-α treatment had a pronounced anti-proliferative effect on CD34⁺ cell proliferation as evident by significantly decreased total cell yield output but accelerated the differentiation into DC with little cell death (FIG. 7A-D). In the presence of the DC induction cytokine mixture, EDHF treated CD34⁺ cells were expanded 25-31-fold, whereas FLT3L +SCF+GM-GM-CSF treated cells were expanded 2.4-3.5-fold. EDHF and FLT3L+SCF+GM-CSF treated cultures supplemented with GM-CSF+IL-4+TNF-α at culture initiation or only during the last 7 days of incubation contained mostly cells resembling typical mature DC (FIG. 7D). EDHF-derived DC was extremely homogeneous in morphology and their appearance was not different from FLT3L+SCF+GM-CSF-treated cells. As illustrated in FIG. 9 the surface phenotype of generated DC cells under all culture conditions was consistent with their dendritic morphology. These mature phenotypes were stable, with DC expressing high levels of surface HLA-DR and maintaining CD1a, CD83, and CD86 expression for 7-10 days. Importantly, the cytokine combination containing optimal concentrations of SCF+FLT3L+GM-CSF (˜2-fold increase) was significantly inferior in inducing total DC cell expansion from cord blood CD34⁺ cell progenitor cells when compared with EDHF treated cultures (˜15-fold increase) (FIG. 7C).

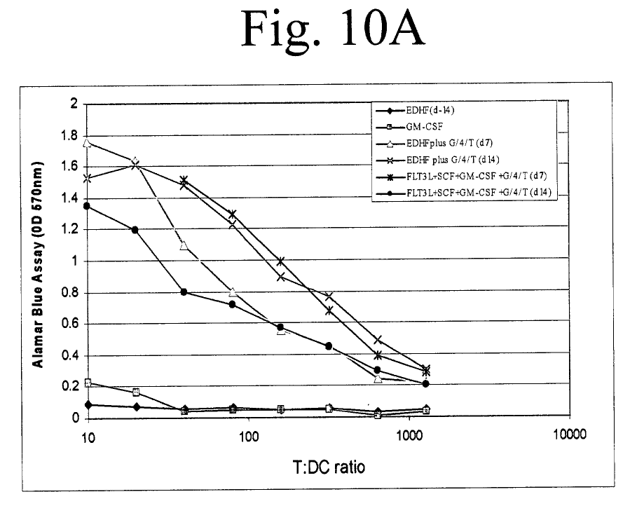

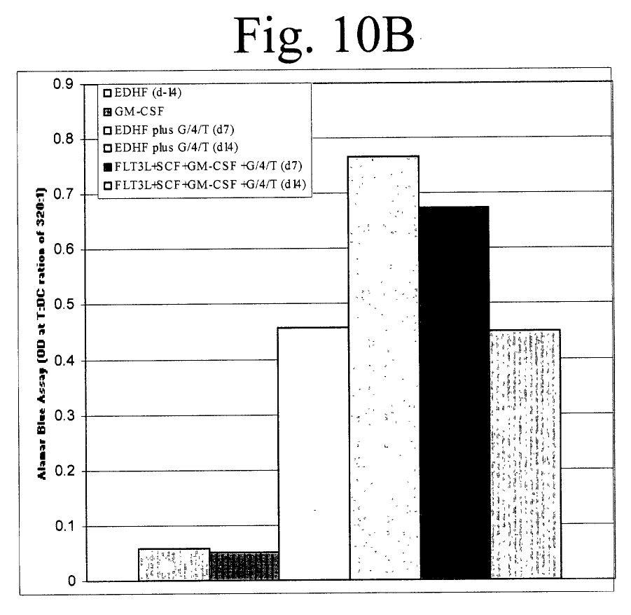

N. Functional competence of EDHF-derived DC. To determine the stimulatory capacity of EDHF-derived DC, graded doses of DC were co-cultured with 5×10⁴ allogeneic naive CD4⁺ T cells, and after 7 days of culture T-cell proliferation and APC function was assessed in the allogeneic MLR using the Almar Blue detection assay system for measuring proliferating cells. A representative experiment is shown in FIG. 10. Neither cells treated with GM-CSF for the last 7 days nor cells treated with EDHF alone for 14 days proved effective for allosensitizing T cells. In contrast, cells from EDHF and FLT3L+SCF+GM-CSF treated cultures supplemented with GM-CSF+IL-4+TNF-α drastically enhanced the T-cell allosensitization capacity. As can be seen from this representative experiment, cells generated in the presence of EDHF are of similar potency on per cell basis in the MLR as those generated from cord blood CD34⁺ cells in the presence of FLT3L+SCF+GM-CSF. Half-maximal CD4⁺ T cell proliferation was detected with 150 to 600 DCs. Less than 50 EDHF-derived DC cells were required for the stimulation of a significant response, in contrast to more than 2,500 GM-CSF treated cells, which contained a modest amount of immature as well as mature granulocytes and macrophages.

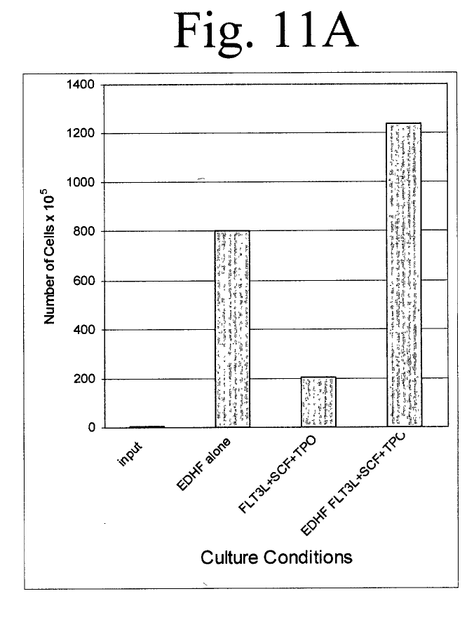

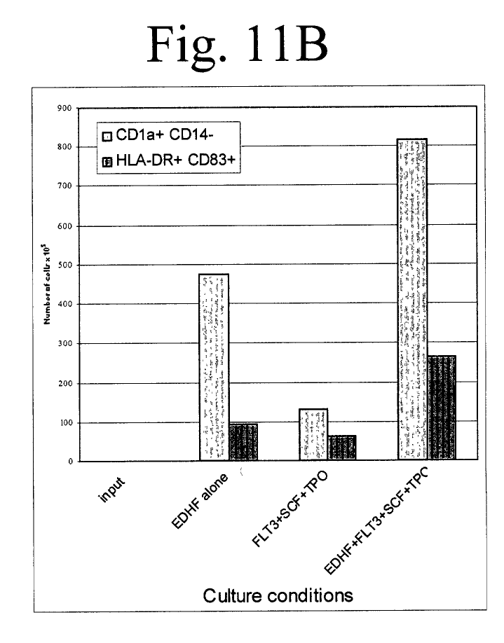

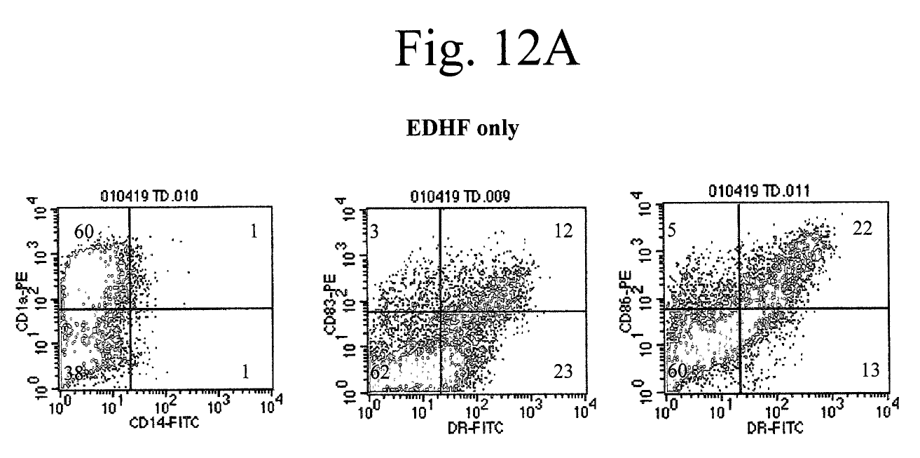

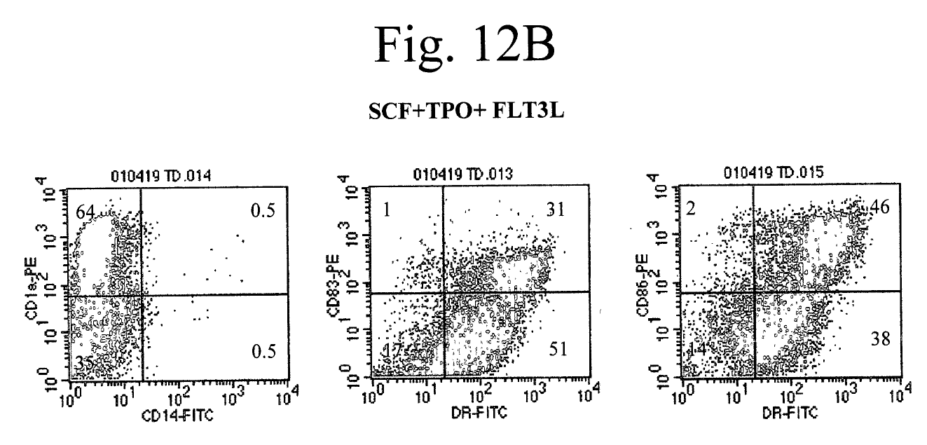

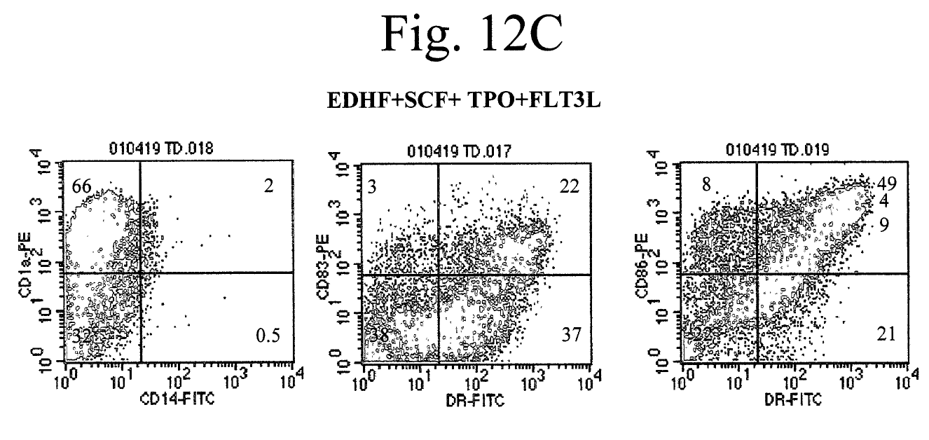

O. Comparison of EDHF versus the cytokine combination of SCF+FLT3+TPO used for the ex vivo expansion of DC precursors from cord blood CD34⁺ cells. A comparison was made assessing the efficacy of DC precursor expansion using EDHF versus the cytokine cocktail of SCF+FLT3L+TPO which has been reported by Arrihi et al, 1999 (Blood 93:2244) to support significant DC precursor expansion for >30 days. Fresh highly purified cord blood CD34⁺ cells were cultured with either EDHF or in the presence of FLT3L+SCF+TPO for 14 days with GM-CSF+IL-4+TNF-α added for the final 7 days of incubation. Cultured cells were harvested, counted, stained for surface marker expression, cytocentrifuged, and stained with Wright-Giemsa. The results shown in FIG. 11 demonstrate that EDHF alone (166-fold cell expansion) is far superior to combinations of FLT3L, SCF, and TPO (42-fold cell expansion) for the generation of DC precursors and DC from cord blood CD34⁺ cells. Neither EDHF alone nor FLT3L+SCF+TPO or EDHF in combination with FLT3L+SCF+TPO was sufficient to induce differentiation of CD34⁺ cord blood cells. Both populations of cells showed an immature morphology without cytoplasmic protrusions. However, when either IL-4+GM-CSF and/or IL-4+GM-CSF+TNF-α were added to the medium a marked increase in HLA-DR, CD1a, CD83, and CD86 expression on cultured cells was detected within 3-4 days (FIG. 12). These generated cells lacked little, if any, CD14 expression. Under these conditions, the cells rapidly formed large aggregates of cells in 2-3 days as previously shown. After an additional 3-5 days of culture, the cells were more dispersed, free floating, and showed multiple long dendrite processes characteristic of DC. Interestingly, the addition of EDHF to optimal and saturating concentrations of SCF+FLT3L+TPO increased total cell (FIG. 11A) and CD1a⁺CD83⁺ cell (FIG. 11B) numbers approximately an additional 55% and 72%, respectively.

### EXAMPLE 2

### Effects on Normal Hematopoiesis in Mice When Administered Soluble Proteins >30 kDa Derived from Porcine Brain Microvascular Endothelial Cells

The following procedures were employed to evaluate whether EDHF would have similar effects on HPC and DC progenitor/precursor cell proliferation, development and/or activation in vivo.

A. Animals: Female mice Balb-C (Harlan Sprague-Dawley, Indianapolis, Ind.) mice (6-8 weeks old) were used for these studies. All mice were provided with acidified water and sterilized rodent chow and housed along with sentinel mice that were routinely screened and shown to be pathogen-free. The BIOCON Animal Care and Use Committee, Rockville, Md. approved all protocols. Research was conducted according to the principles enunciated in the “Guide for the Care and Use of Laboratory Animals” prepared by the Institute of Laboratory Resources, National Research Council, Washington, D.C.

B. EDHF administration Protocol: Mice were injected subcutaneously (s.c.) once daily for 7 days with 200 μL of EDHF (70×final concentration, >30 kD MW, from lot#070500). Control mice were injected with phosphate-buffered saline (PBS) plus 1% bovine serum albumin (BSA). All mice were sacrificed the day after the last injection and blood was drawn for assessing the number of clonogenic progenitors (CFU-C). Peripheral blood was obtained by cardiac puncture using a heparinized syringe following carbon dioxide (CO₂) asphyxiation. Blood was transferred to tubes containing ethyelenediamine tetraacetic acid (EDTA) for WBC analysis and differential counts were determined from Wright-stained smears. Peripheral blood mononuclear cells were isolated by underlaying 400 μL of blood diluted in 3 volumes of PBS with Ficoll-Hypaque (Sigma, St. Louis, Mo.) and by centrifugation at room temperature at 400 g for 30 minutes. Contaminating erythrocytes (RBCs) were lysed in 0.8% NH₄Cl and the remaining nucleated cells were washed thrice in Iscove's IMDM containing 1% BSA. Bilateral femora and spleen were taken and the spleen weight was recorded. BM cell suspensions were obtained by flushing the bones with 1 mL of Iscove's modified Dulbecco's medium (IMDM) supplemented with 1% bovine serum albumin (BSA) (Sigma, St Louis, Mo.). Spleen cell suspensions were prepared by mincing the tissue with scissors, passing it through a 21-gauge needle, and then filtering through a 70-μm nylon cell strainer (Becton Dickinson, Franklin Lakes, N.J.). Bone marrow and spleen mononuclear cell suspensions were isolated by Ficoll-Hypaque (Sigma, St. Louis, Mo.) separation. Cells at the interface were removed and washed twice with phosphate-buffered saline containing 1% BSA. After the last wash, the cell pellet was suspended in IMDM containing 1% BSA. Nucleated cells were counted on a hemocytometer using the trypan blue dye exclusion assay. In all experiments, the number of mice per group was at least 5. Summarized results are from duplicate experiments with 10 animals per group unless otherwise noted.

C. Flow cytometry: A total of 5×10⁵ cells were incubated for 30 minutes at 4° C. with fluorescein isothiocyanate (FITC)- or phycoerythrin (PE)-conjugated monoclonal antibodies; FITC conjugated I-A/I-E (clone 2G9), CD11C-PE (clone HL3) and CD86 (B 7.2, clone GL1) all obtained from BD/Pharmingen, San Diego, Calif. Appropriate conjugated isotype-matched antibodies were used as controls. In all experiments, cell samples were preincubated in 0.1 mL PBS supplemented with blocking reagent, either human IgG (Bayer, Elkhart, Ind.) or BD FC block reagent (BD/Pharmingen, San Diego, Calif.) for 30 minutes to block nonspecific FC binding. After a wash with PBS, cells were stained for 30 minutes on ice with various monoclonal antibodies (mAbs) conjugated by fluorescein isothiocyanate (FITC) or phycoerythrin (PE). Stained cells were washed with staining buffer twice (D-PBS supplemented with 0.2% BSA), fixed with 0.5 mL of 1.6% paraformaldehyde (Electron Microscopy Sciences, Fort Washington, Pa.) and expression of cell-surface antigens specific for mAb was determined by using a fluorescence-activated cell-sorter scanner (FACS) flow cytometer and CellQuest software (Becton Dickinson, San Jose, Calif.). 5,000 cells were analyzed through a viable cell gate as determined by forward and right angle light scatter parameters to exclude subcellular particles. The cytometer was calibrated utilizing manufacturer supplied Autocomp beads and software.

D. Murine clonogenic assays: CFCs, including colony-forming unit GM (CFU-GM), mixed multilineage colony-forming unit (CFU-GEMM, macrophage colony-forming unit (CFU-M) and burst-forming unit-erythroid (BFU-E) were estimated by the standard methylcellulose method using 35 mm culture dishes (Costar, Corning, Pa.). One-milliliter suspensions of 5×10⁵ nucleated PB cells, 1 to 2×10⁴ nucleated marrow cells, or 5×10⁴ to 1×10⁵ nucleated spleen cells were plated in triplicate. One volume of hematopoietic cells was added to 9 volumes of murine Methocult M3434 Media (StemCell Technologies, Vancouver, BC, Canada). The plates were incubated for 7 days at 37° C. in a fully humidified 5% CO₂-air atmosphere, and colonies containing more than 50 cells were scored using an inverted microscope. Colony-forming cells (CFC) were scored on days 7-8 using an inverted microscope (×40 magnification). Morphological verification of selected colonies was determined using Wright-Giemsa stain. CFC numbers were then corrected to obtain the number of CFC either per milliliter of peripheral blood and total CFC content per spleen and femur.

E. In vivo Investigation: To examine EDHF-induced hematopoietic progenitor cell (HPC) expansion and mobilization in vivo, 6-8 week aged matched female Balb-C mice (n=10) were treated with EDHF alone (one dose per day, s.c. administration, 200 μL of EDHF, 70×concentrate, >30 kD MW) for 7 consecutive days and measurements were taken for total peripheral blood, splenic and bone marrow progenitor cell content in addition to the evaluation of DC numbers in both spleen and bone marrow cell populations. Control mice were injected with phosphate-buffered saline (PBS) plus 1% bovine serum albumin (BSA).

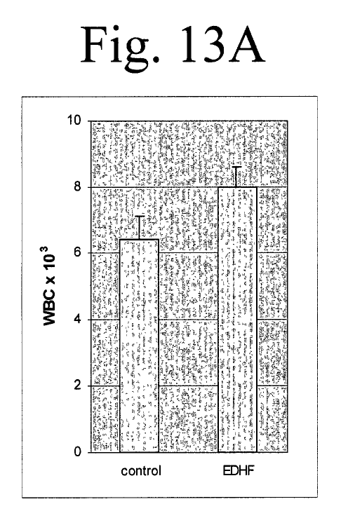

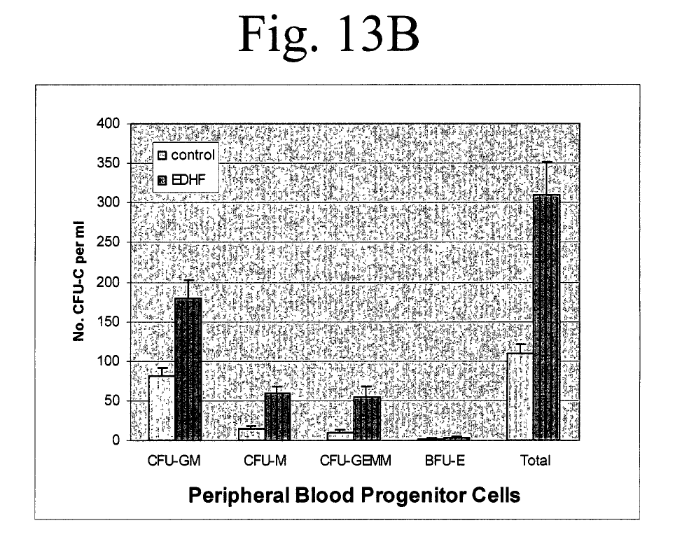

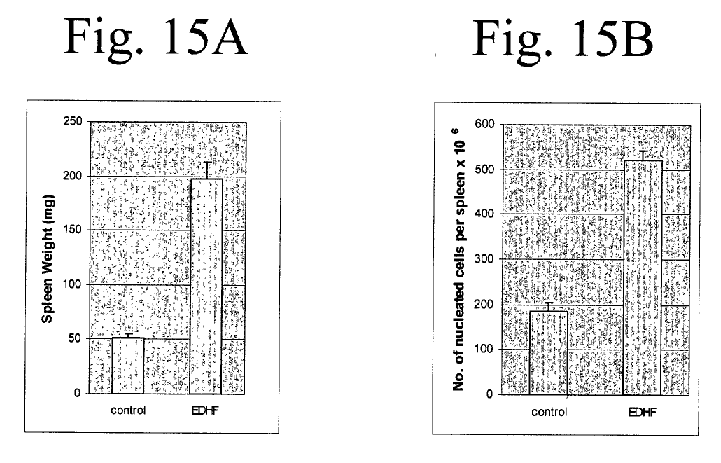

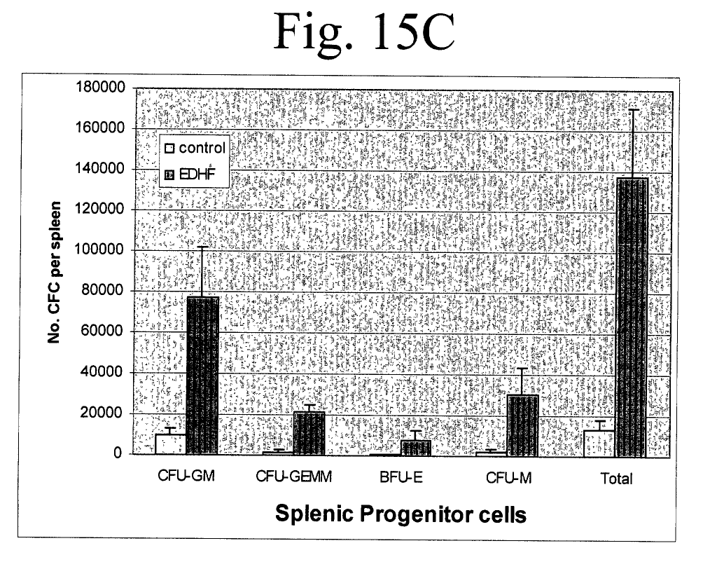

F. EDHF stimulates HPC mobilization into peripheral blood: White blood cell (WBC) counts (FIG. 13A), spleen weight (FIG. 15A), and the number of CFC in peripheral blood (FIG. 13B) were evaluated as indicators of HPC mobilization. Using the outlined dosing regimen, EDHF alone had a minor impact on circulating WBC levels (25% increase). Peripheral blood lymphocyte, monocyte, neutrophil, eosinophil and basophil levels were within normal range. EDHF administration resulted in a modest increase (2.8-fold) in CFC numbers in the peripheral blood (FIG. 13B). Although EDHF mobilized predominantly CFU-GM progenitor cells (2.2-fold increase), significant increases in CFU-GEMM (5.7-fold) and CFU-M (4.1-fold) progenitor cells were also detected. In contrast, EDHF treatment had no effect on BFU-E progenitor cell numbers when compared to controls.

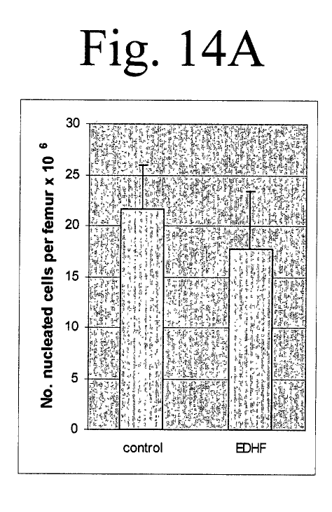

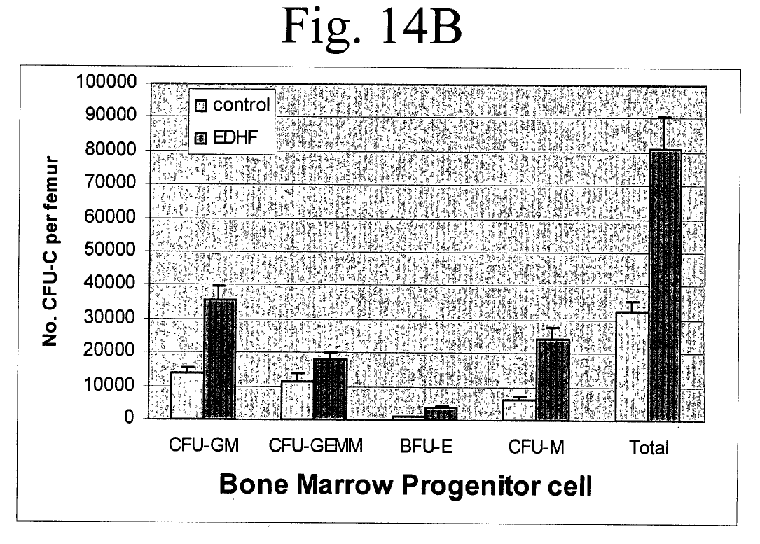

G. Distribution of HPC progenitor cells in the bone marrow and spleen following EDHF administration: The number of progenitor cells within the MNC population harvested from control and EDHF-treated mice was assessed by a clonogenic assay for CFU-C. After 7 days of EDHF administration, the number of bone marrow MNC cells per femur had decreased by 18% (FIG. 14A). However, as shown in FIG. 14B, the percentage (1.4% in controls and 4.6% EDHF treated) and the absolute number of total CFU-C increased 250% in the bone marrow of EDHF treated mice. In comparison to the bone marrow progenitor cell content in control mice, CFU-GM, CFU-GEMM, CFU-M and BFU-E progenitor cells increased 250%, 150%, 390% and 302%, respectively, following 7 days of EDHF administration.

Administration of EDHF alone for 7 days induced a marked splenomagaly (FIG. 15A) as evidenced by increased spleen weight from 51.1±4.7 mg to 197.6±15.8 mg. Moreover, EDHF alone for 7 days and increased nucleated cell counts 283% (mean 522×10⁶ cells/spleen versus 184×10⁶ cell/spleen in control treated animals) as illustrated in FIG. 15B. As shown in FIG. 15C, the percentage (0.007% in controls and 0.03% EDHF-treated) and the absolute number of total splenic CFU-C progenitor cells increased 1,031% following 7 days of EDHF administration with an 1,000%, 1,370%, 1,560% and 2,005% increase in CFU-GM, CFU-GEMM, CFU-M, and BFU-E progenitor cells, respectively.

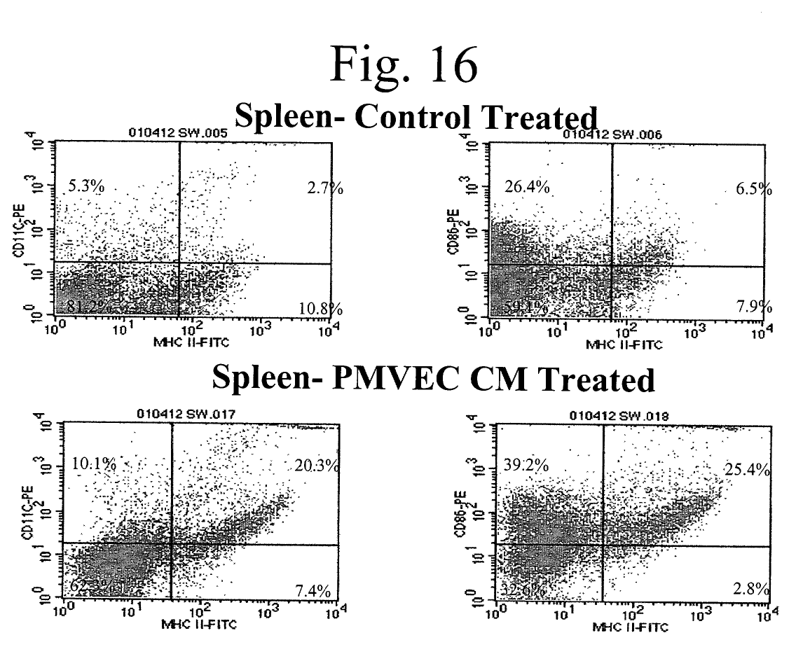

H. Administration of EDHF to mice results in large increases of CD11c⁺MHC II⁺CD86⁺ cells in the spleen but not bone marrow: Using flow cytometeric analysis we found that splenic MNC cells expressing the CD11C⁺ phenotype increased significantly from 3% in control animals to approximately 20% in mice administered EDHF for 7 days (FIG. 16). Greater than 67% of these splenic-derived CD11C⁺ cells expressed high levels of MHC class II and CD86 costimulatory molecules, which appear to be phenotypically similar to myeloid-related dendritic cell subsets, previously identified in the peripheral blood and lymphoid tissues of normal and FLT3L-treated mice. As shown in FIG. 17, EDHF treatment alone resulted in 17.6-fold increase in the absolute number of DC per spleen. In contrast, we detected no significant change in CD11C⁺ cell numbers in the bone marrow following 7 days of EDHF administration when compared to control treated animals.

I. Summary: These data demonstrate that EDHF alone is a potent stimulus for the proliferation and expansion of HPC in mice. As a single agent, EDHF elevated the absolute numbers of clonogenic progenitor cells in the bone marrow (3-fold) and spleen (10-fold). The dose and administration schedule employed led to a modest mobilization of progenitor cells into the circulation and redistribution of progenitor cells to the spleen without an associated decline in marrow cellularity and CFC progenitor cell content. In fact, EDHF led to an increased progenitor cell concentration in the bone marrow. Typically, cytokine based mobilization protocols are accompanied by a marked loss of total cells and both progenitor and primitive stem cells in the marrow. It is therefore likely that the observed splenomegaly resulted from a low level of migration of expanded HPC cells from the marrow to the spleen and from further proliferation of these cells in the spleen. The marked expansion of myeloid progenitor cells in both the spleen and bone marrow incurred without a concomitant increase in circulating neutrophil and monocytes. It has been previously reported that dosing regimens using G-CSF, GM-CSF or various combinations of both molecules typically results in a dramatic and prolonged elevation in circulating WBC levels, associated with increased neutrophil counts (>10 fold). EDHF is potent in its capacity to stimulate cells of the DC lineage in mice. In this example, it is demonstrated that when administered subcutaneously to a mammal, EDHF dramatically increases CD11C⁺ MHCII⁺ CD86⁺ cells in the spleen, which are phenotypically similar to the myeloid-related dendritic cell subset.

### EXAMPLE 3

### Endothelial Cell-Derived Hematopoietic Growth Factor (EDHF) Expands Murine Hematopoietic Progenitor Cells and DC Precursor Cells In vivo and Increases the Protective Response to Autologous Tumor Vaccination

An appealing alternative to multichain whole Ig vaccines is singe-chain variable region (scFv) vaccines. Consisting of just the hypervariable domains from the tumor-specific Ig, these proteins recreate the antigen-binding site of the native Ig and are a fraction of the size, and can be expressed in several expression systems, including transgenic plants. scFv vaccines, either as protein or DNA, are capable of eliciting anti-idiotype-specific responses in animals and are effective in blocking tumor progression in mouse models of lymphoma.

A modified tobamoviral vector was made that encodes the idiotype-specific single-chain Fv fragment (scFv) of the immunoglobulin from the 38C13 mouse B cell lymphoma. Infected Nicotiana benthamiana plants contain high levels of secreted scFv protein in the extracellular compartment. This material reacts with an anti-idiotype antibody by Western blotting, ELISA, and affinity chromatography, suggesting that the plant-produced 38C13 scFv protein is properly folded in solution. Mice vaccinated with the affinity-purified 38C13 scFv generate >10 μg/ml anti-idiotype immunoglobulins. These mice were protected from challenge by a lethal dose of the syngeneic 38C13 tumor, similar to mice immunized with the native 38C13 IgM-keyhole limpet hemocyanin conjugate vaccine. This rapid production system for generating tumor-specific protein vaccines may provide a viable strategy for the treatment of non-Hodgkin's lymphoma.

Bacterial DNA is capable of inducing activation of B cells, NK cells, monocytes and can induce production in vitro and in vivo of a variety of proinflammatory cytokines. In contrast, vertebrate DNA does not induce lymphocyte activation. Bacterial DNA contains a much higher frequency of unmethylated CpG dinucleotides than does vertebrate DNA, which may represent an immune defense mechanism that can distinguish bacterial from host DNA. Select synthetic oligodeoxynucleotides contain unmethylated CpG motifs (CpG ODN) have immunostimulatory effects similar to those seen with bacterial DNA. Immunostimulatory oligodeoxynucleotides containing the CpG motif (CpG ODN) can induce production of a wide variety of cytokines and activate B cells, monocytes, dendritic cells, and NK cells.

A. Tumor Model

Dendritic cells (DC) are potent antigen processing and presenting cells considered to be essential for initiating rapid and efficient immune responses, and possess the unique ability to stimulate naive T-cells and B-cells. Increasing vaccine potency by stimulating antigen uptake and presentation by DC is desirable. In the previous example, we demonstrated that treating mice with porcine endothelial cell-derived hematopoietic growth factor (EDHF) comprising proteins >30 kDa stimulates hematopoietic progenitor cell expansion and mobilization and produces a 17.6-fold increase in splenic DC numbers. To evaluate the effect of EDHF on vaccine potency (enhancing the effects of a vaccine), 6-8 week old, aged-matched female C3H/HEN mice (n=10) were pretreated with EDHF alone, one dose per day, subcutaneously s.c., 200 μl) for 7 consecutive days. Then, vaccine groups (EDHF pretreated mice and mice that were not pretreated) received 15 μg of protein derived from the 38C13 mouse B-cell lymphoma (a tumor-associated syngeneic self-antigen protein), s.c. at 2-week intervals for a total of two vaccinations. To ensure activation of DC at the site of vaccine injection, the vaccine was mixed with either control vehicle or 10 μg of CpG DNA oligomer (Hartman, et al., PNAS 1999, 96(16): 9305-10). Ten days after each vaccination, humoral anti-idiotypic immunoglobulin levels were determined by ELISA.

B. ELISA Determination of Anti-Id Levels

Serum was obtained by retroorbital puncture. Microtiter plates were coated with 5 μg/ml 38C13 IgM in carbonate buffer overnight. IgM-coated plates were blocked with 2.5% BSA+2.5% milk in PBS, and serial dilutions of serum were added. A known concentration of monoclonal anti-Id served as a standard. Plates were washed, and heavy chain-specific goat anti-mouse IgG, IgG2a or IgG2b conjugated to horseradish peroxidase (HRP; Southern Biotechnology Associates, Birmingham, Ala.) was added. HRP activity was detected by adding the colorimetric substrate p-nitrophenylphosphate with hydrogen peroxide in citrate buffer by standard method. Plates were evaluated using a microplate reader. Values were considered valid if they fell within the linear range of the standard curve.

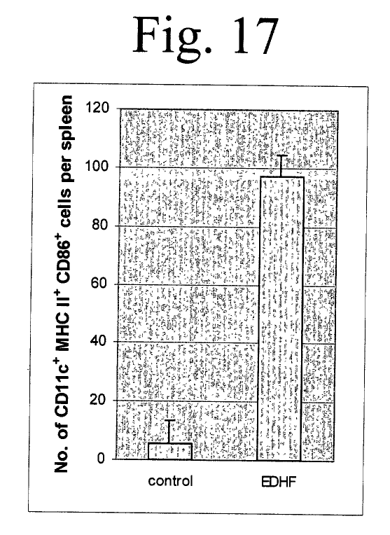

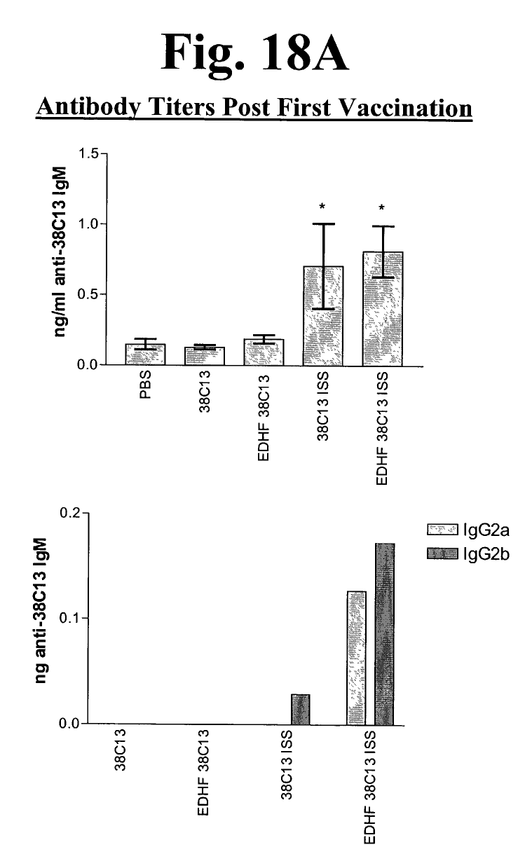

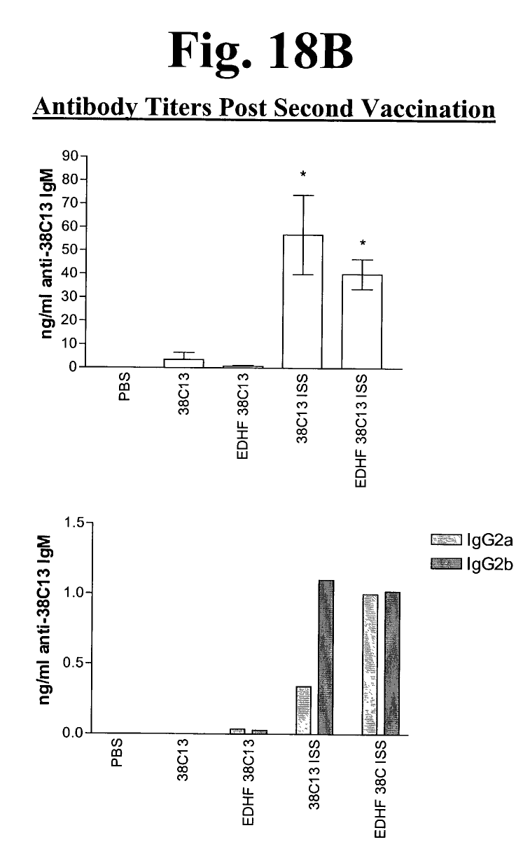

A pronounced anti-38C13 immune response was detected as early as 10 days following the first vaccination of the group pretreated with EDHF compared to control groups, which had little or no detectable response. Isotype analysis revealed a predominantly IgG2 (IgG2a and IgG2b combined) response after a single vaccination, suggesting early and robust Th1-type B-cell help characteristic of dendritic cell antigen presentation (FIG. 18A). scFv+ CpG DNA, on the other hand without EDHF pretreatment, gave only IgG1 isotype response, suggesting less effective, or nondendrtic cell antigen presentation. After two vaccinations, mice treated with EDHF+vaccine, or vaccine alone in the absence of CpG immunization, had significantly lower serum anti-38C13 titers with little IgG2 isotype (FIG. 18B). Its known that, murine IgG2a is more effective than murine IgG at mediating antibody-dependent cellular cytotoxicity, and monoclonal IgG2a works better than monoclonal IgG with the identical variable region as a set of therapeutic antibodies for treating tumors in mice (Kaminski, M. S., J. Immunol. 136:1123-1130, 1986).

C. In vivo Survival Studies Following Tumor Challenge

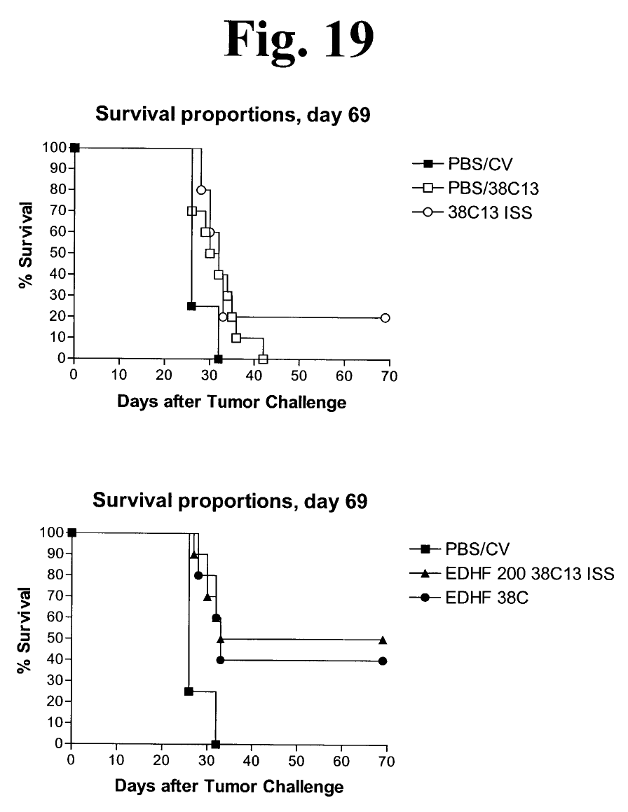

Two weeks after the second vaccination, animals were challenged with a lethal dose of antigen-expressing 38C13 lymphoma tumor cells (subcutaneous injection of 1000 viable cells). 38C13 tumor cells for injection were growing in log phase for at least 3 days prior to inoculation. Mice that developed tumor displayed inguinal and abdominal masses, and cachexia. All mice that developed tumor died. Survival was monitored for 70 days, and significance with respect to time to death was assessed using log rank regression analysis. Control mice, receiving PBS pretreatment and no vaccine, all succumbed to tumor within 30 days of challenge (FIG. 19). Mice given vaccine alone, or vaccine with ISS were not statistically different that the control group. Animals pre-treated with EDHF followed by vaccine+CpG DNA vaccination had significantly better survival than controls, vaccine treatment alone, or vaccine+CpG. Mice given EDHF pretreatment and protein vaccination, either with or without CpG DNA, survived tumor challenge at 50 and 40% respectively. Comparison of survival curves between EDHF pretreated groups to control or PBS pretreated groups showed statistical significance (P=0.0075 with CpG, P=0.0298 without CpG DNA). These results show that in vivo expansion of DC precursors through administration of EDHF augments vaccine potency by increasing antigen uptake and antigen presentation. These results represent an important strategy for increasing the effectiveness of vaccination without modification of the antigen and without purification of DC.

In further embodiments human endothelial proteins having a molecular weight of >30 kDa are employed as EDHF in examples substantially similar to Examples 1, 2 and 3 (with the exception that human derived EDHF is used in place of porcine EDHF) to achieve the same results. In additional embodiments, IGFBPs, and in particular IGFBP-3, made recombinantly, are used as EDHF in both in vitro and in vivo applications as described herein.

### EXAMPLE 4

### Engraftment Potential of ex vivo Cultured Cord Blood CD34⁺ Cells Treated with EDHF

A. Porcine microvascular endothelial cells: Primary porcine brain microvascular endothelial cells (PMVEC) (passages 26-35) were maintained in endothelial cell culture medium consisting of M199 medium (GIBCO LIFE Technologies, Gaithersburg, Md.) supplemented with 10% FBS (Hyclone, Logan Utah), 30 μg/mL endothelial cell growth factor supplement (Sigma, St. Louis, Mo.), 100 μg/ml L-glutamine, 100 U/ml penicillin/streptomycin solution, and 50 μg/ml preservative-free sodium heparin (Sigma, St. Louis Mo.)) and passaged weekly at 1×10⁶ cells per gelatin-coated 75 cm2 flask.

B. Production of EDHF conditioned medium: For the production of EDHF conditioned medium, PMVEC cells were grown to confluence in PMVEC endothelial cell growth medium consisting of M199 supplemented with 10% heat-inactivated FCS, 50 μg/ml preservative-free heparin, 100 μg/ml L-glutamine, and 100 U/mL penicillin/streptomycin solution, washed twice with PBS, and refed Iscove's (IMDM) medium without serum. After 7 days of culture, conditioned medium was harvested, filtered through a 0.2 um membrane to remove cell debris, and proteins >30 kDa concentrated 70× by ultra filtration using an YM-30 Amicon membrane. The concentrated EDHF was passed through a 0.2 μm filter, aliquoted, and stored at −20° C. All batches of EDHF were tested for their ability to support human CD34⁺ hematopoietic cell proliferation.

C. Isolation of CD34⁺ hematopoietic progenitor cells: Human cord blood (CB) was obtained during normal full-term deliveries after informed consent was given. CB samples (50-150 mL) were diluted 1:4 with Dulbecco phosphate-buffered saline (DPBS) Ca⁺⁺ and Mg⁺⁺-free (GIBCO-BRL, Grand Island, N.Y.). Diluted CB was then underlaid with Ficoll-Paque (Pharmacia AB, Uppsala, Sweden), and centrifuged at 800 g for 30 minutes at 20° C. The mononuclear cell fraction was collected and the CD34⁺ cells were immunomagnetically enriched using the MACS CD34 Isolation Kit (Miltenyi Biotec, Auburn, Calif.). Procedures were performed as per manufacturer's recommendations. Cells were incubated with hapten-labeled anti-CD34 antibody (QBEND-10, Becton Dickinson) in the presence of blocking reagent, human IgG (Bayer, Elkhart, Ind.), and then with antihapten coupled to MACS microbeads. Labeled cells were filtered through a 70 μm nylon mesh and separated using a high-gradient magnetic separation column. Magnetically retained CD34⁺ cells were eluted following several washes of the column with D-PBS. The purity of the CD34⁺ population was routinely more than 90%. CD34⁺ cells were either used for experimentation or cryopreserved in 10% dimethylsulfoxide (Sigma), 50% fetal calf serum (FBS, Hyclone Laboratories, Logan, Utah) by controlled-rate freezing methods. Following thawing, samples were usually pooled to provide sufficient cell numbers for each experiment.

D. Ex vivo expansion cultures: A total of 2.3×10⁶ purified human CB CD34⁺ cells were seeded at cell density of 2×10⁵ CD34⁺ cells per tissue dish (35 mm; Corning, Corning, N.Y.) in 3 mL RPMI 1640 medium (GIBCO-BRL, Grand Island, N.Y.) containing 2 mmol/L L-glutamine, 10 mmol/L HEPES, 50 IU/mL penicillin, 125 μg/mL streptomycin, 10% fetal bovine serum (FBS; Hyclone, Logan, Utah) and 50 mM ME. Cultures were placed at 37° C. in 100% humidified atmosphere of 5% CO₂ in air. Cultures were treated with a previous determined optimal concentration of EDHF (1:70 dilution, 1×final concentration of the 70×stock >30 kD MW, Lot# 070500). After 7 days of culture, nonadherent cells were harvested from each culture dish, pooled, washed 2×with D-PBS and resuspended in fresh medium containing EDHF to suppress any type of growth factor dependent apoptosis prior to infusion of cells into SCID/NOD mice. The total viable cell yield was determined by trypan blue dye exclusion (Sigma, St. Louis, Mo.).

E. Xenotransplantation of human hematopoietic cells: NOD/SCID mice (female, 8-10 weeks of age) were purchased (Jackson Laboratory, Bar Harbor, Me.) and maintained in micro-isolator cages and provided with autoclaved food and water. Mice were irradiated with 350 cGy of ¹³⁷Cs and thereafter received acidified water containing 100 mg/L ciprofloxacin (Bayer AG, Leverkusen, Germany). Test cells (100,000 and 300,000 cells per graft) were injected intravenously within 4-6 hr after the mice were irradiated. No exogenous human growth factor or EDHF CM was administered. After 5 weeks, mice were killed, and bone marrow was collected from both femurs. Bone marrow cells were harvested by flushing the femur bones with PBS/2% FBS using a 3-mL syringe and a 21-gauge needle. The cell suspension was washed once and then resuspended in PBS/2% FBS. Cells were counted (using trypan blue to exclude dead cells) and assayed by flow cytometry and CFC assays to determine the level of human cell engraftment. Human cell content of the bone marrow was quantified by flow cytometeric analysis of the human-specific pan-leukocyte marker CD45 and human-specific CFC progenitor cell assays were conducted to determine the level of human progenitor cell engraftment in the murine bone marrow compartment. Only mice with >1% total human cell content and whose marrow contained human CFC progenitors were considered to be engrafted.

F. Cell surface phenotyping and microscopic analysis: Irrelevant, isotype-controlled antibodies were used in every experiment to determine background staining. For detection of human cells in mouse bone marrow, a perCP-labelled anti-CD45 antibody (HLe-1; Becton Dickinson) was used. To further define the different lineages in the total human cell population, bone marrow cells were simultaneously stained with anti-CD45-perCP and an antibody against one of the following human cell lineage markers: CD14-PE, CD19-PE, CD33-PE, and CD34-PE, CD14-PE (all from Becton Dickinson). As part of the analysis of human cell engraftment in mice, bone marrow cells from control NOD/SCID mouse were labeled to ensure that the Abs used were specific for human cells. All staining procedures were performed in PBS/2% FBS. Cell labeling was performed on ice (35 minutes), after which contaminating RBC were lysed using NH4CL for 5 min. Cells were then washed twice and fixed with 0.5 mL of 1.6% paraformaldehyde (Electron Microscopy Sciences, Fort Washington, Pa.). Flow cytometric analysis was performed on a FACScan (Becton Dickinson). A total of 10,000-gated cells were examined and analyzed using Cell Quest Software (Becton Dickinson). Results are expressed as percent positive cells after subtracting negative control values.

Wright-Giemsa staining was performed on cytocentrifuged cell preparations of freshly isolated and cultured cord blood CD34⁺ cells. Cells were suspended in 40% FBS at 1×10⁵ cells/mL. One hundred microliters of cell suspension was spun onto glass microscope slides. Slides were air-dried, cells methanol fixed, and stained with Wrights-Giemsa stain. Cells were visualized and representative cells were photographed using phase 50×high dry hematology lens (×500 magnification, Olympus Optics, Mellville, N.Y.).

G. Human colony-forming cell assay: Murine bone marrow cells from control (no transplant) and transplanted mice were assayed for human colony forming cells (CFC) progenitor content. Cells were plated in semi-solid methylcellulose medium containing optimal amounts of human IL-3, SCF, and GM-CSF and EPO (Methocult GF H4434; Stem Cell Technologies Inc, Vancouver, BC, Canada) at a concentration of 5-10×10⁴ cells/culture dish, and incubated for 14 days. Duplicate cultures for each measurement were established and analyzed. CFU-GM, CFU-GEMM, and BFU-E colonies (>50 cells) were counted by visual examination of the plates, according to standard methods. Results are expressed as the mean colony count per 10⁵ cells plated and the total number of human CFC progenitor cells contained within both femurs. Control dishes containing bone marrow cells from nontransplanted NOD/SCID mice did not support the growth of murine hematopoietic progenitor cells (no colony formation) under these culture conditions.

H. Fluorescence microscopy: An Olympus BX-40 System Microscope equipped with a SPOT RT Color CCD camera (Diagnostic Instruments, Inc.) was employed to evaluate the phenotype of cells derived from human colony-forming progenitor cells (CFC assay) cultured in methylcellulose-based medium supplemented with growth factors specific for human CFC growth and development.

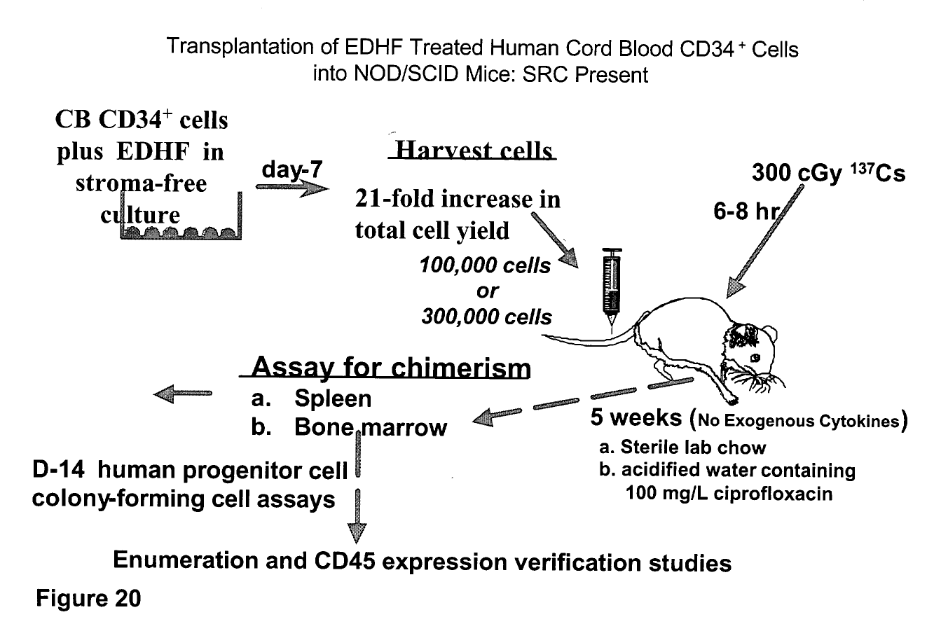

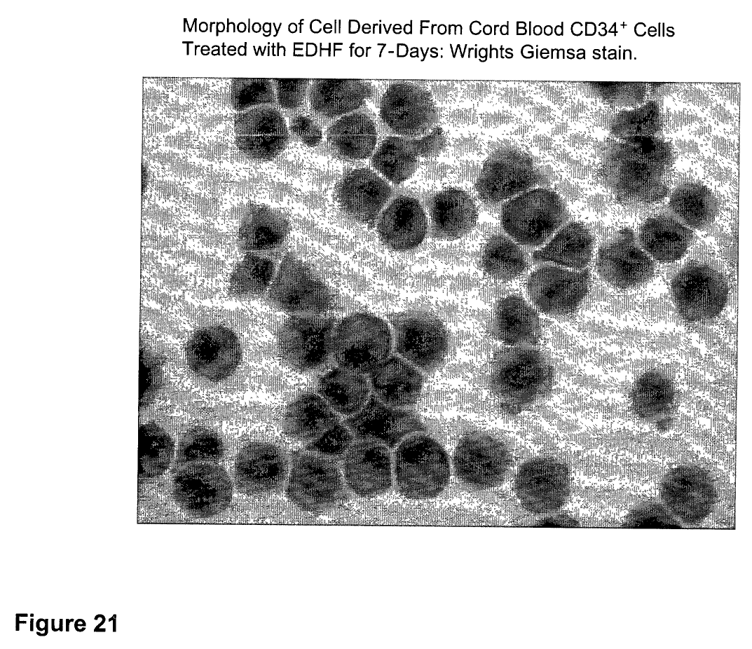

I. Effect of EDHF on CD34⁺ cell proliferation and ex vivo cell expansion: FIG. 20 outlines the experimental scheme. Purified human cord blood-derived CD34⁺ hematopoietic cells (2.3×10⁶ cells) expanded 21.7-fold (50×10⁶ total cells harvested) during a 7-day culture interval with an optimal concentration of EDHF. Some of these cells were cryopreserved and phenotypic analysis of these cells will be conducted in the near future. The morphology of the cells transplanted are shown in FIG. 21 (Wright-Giemsa cytocentrifuge cell preparation).

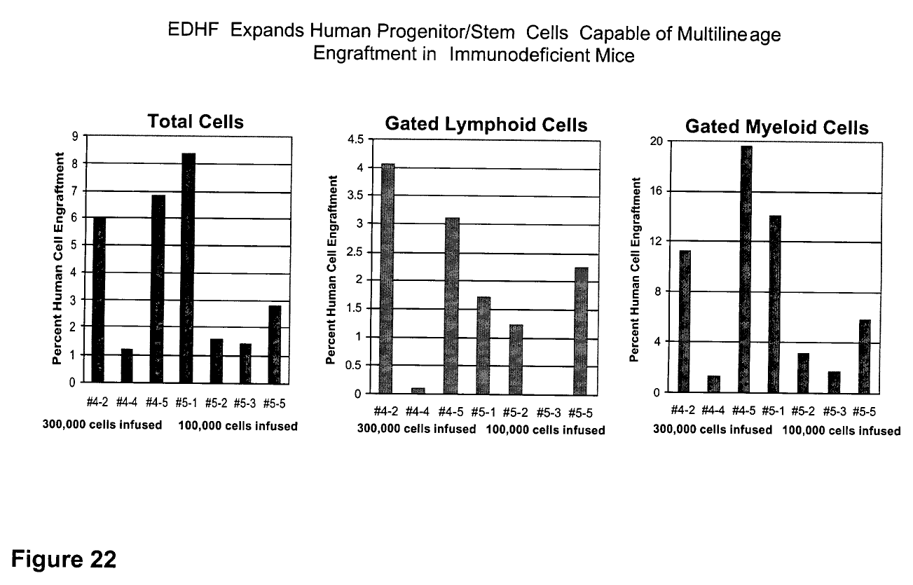

J. Effect of EDHF treatment on human cell engraftment: Groups of sublethally irradiated SCID/NOD mice were transplanted with either 1×10⁵ or 3×10⁵ ex vivo cultured cells, which corresponds to 0.2% and 0.6%, respectively, of the total ex vivo generated cell population. Animals not transplanted served as appropriate controls. Human cell engraftment in the murine bone marrow compartment was examined after 5 weeks by flow cytometry using a human-specific CD45 antibody. No significant numbers of human cells were detectable in the spleens of any group (data not shown). The cellularity of the bone marrow in all recipients was within range of the nontransplanted control mice. As expected, no detectable level of human CD45 expression was measured in the bone marrow of nontransplanted control animals (range 1.2% to 8.5% human cell engraftment). As shown in FIG. 22, 60-80% of the five mice in each group that received ex vivo cultured cells had significant levels (>1%) of human CD45⁺ cell engraftment in the murine bone marrow compartment. The proportion of human CD45⁺ lymphocytes, myeloid cells and CD34⁺ cells present in mice transplanted with ex vivo cultured cells was determined. The frequency of both lymphoid (0.2 to 4.1%) and myeloid engraftment (0.4 to 19.7%) was greater in those animals transplanted with 3×105 cells. CD19⁺ B-cells were the major human component of the lymphoid compartment, whereas CD33⁺ cells made up the majority of the myeloid compartment, which contained none to very few detectable human CD14⁺ monocytes/macrophages. Most of the engrafted mice contained a modest number of human CD34⁺ cells in the lymphoid region (range 0.2 to 1.5% CD34⁺ cells). The femoral bone marrow of mouse #5-5 contained 14.6×10⁶ bone marrow cells, of which 2.9% were human. Therefore, 0.42×10⁶ human cells were derived from 1×10⁵ input cells, representing an increase of at least 4.2-fold (with a femur representing only 10-15% of the total bone marrow compartment).

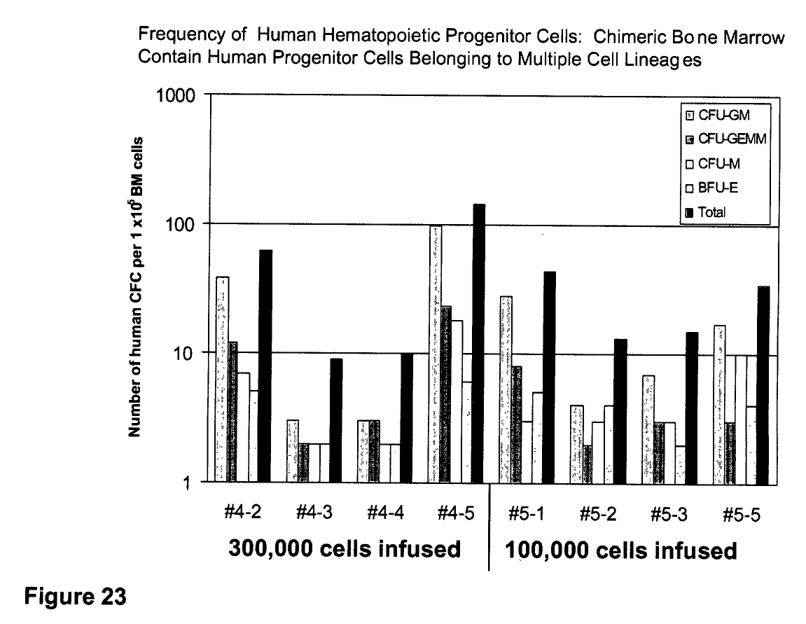

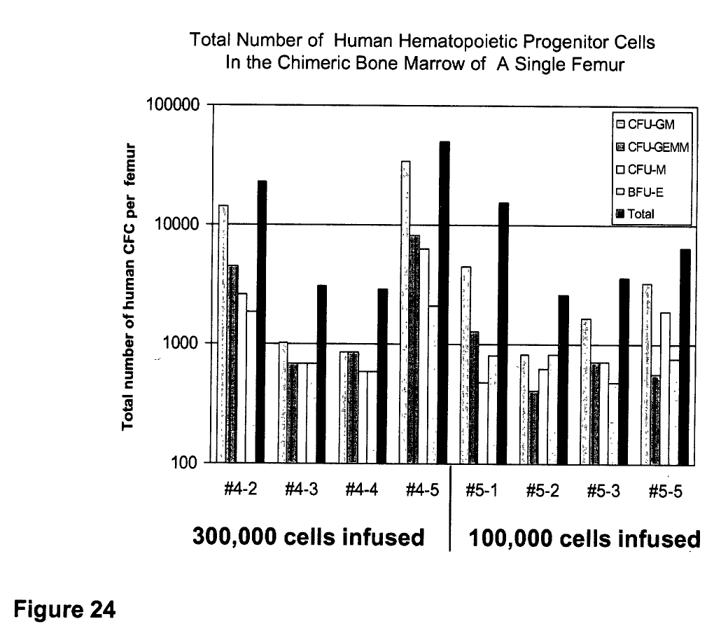

Using the CFC assay, we were able to detect significant human progenitor cell engraftment in the bone marrow of 80% of the mice transplanted with low numbers of EDHF treated CD34⁺ cells (FIGS. 23 and 24). Human progenitor cell engraftment was detected in mouse #4-3, wherein no detectable (>1%) human CD45⁺ cell engraftment was detected when bone marrow cells were analyzed via FACS. The level of human CFC progenitor cell content of the two groups correlated with the level of human cell engraftment measured via FACS analysis. Calculations of the total number of CFU-GM, CFU-GEMM, CFU-M, and BFU-E CFC progenitor cells present in the femurs of these mice confirmed the results of higher levels of human cell engraftment in the group transplanted with 3×10⁵ cells per graft.

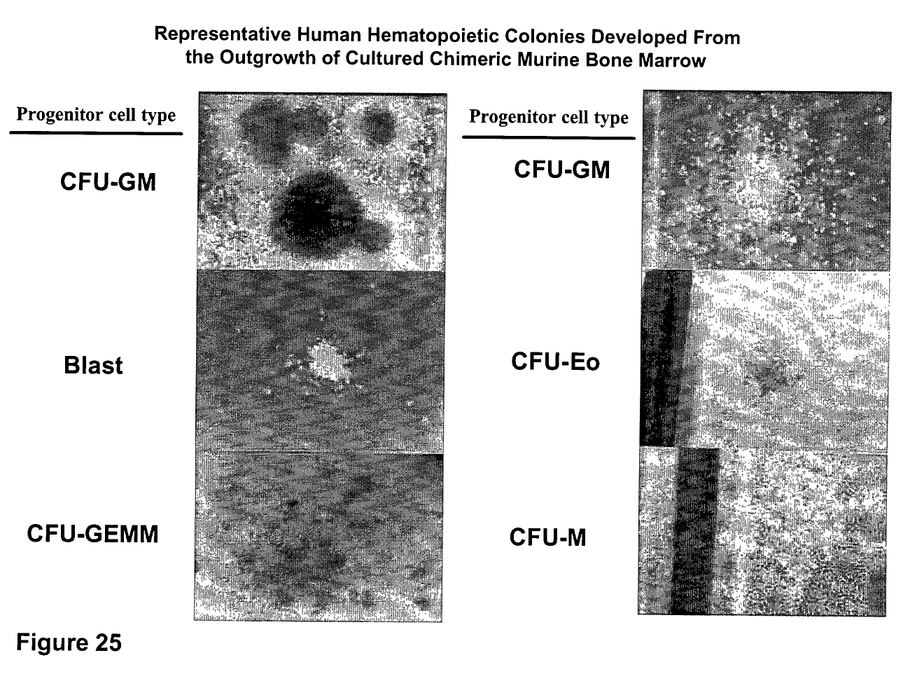

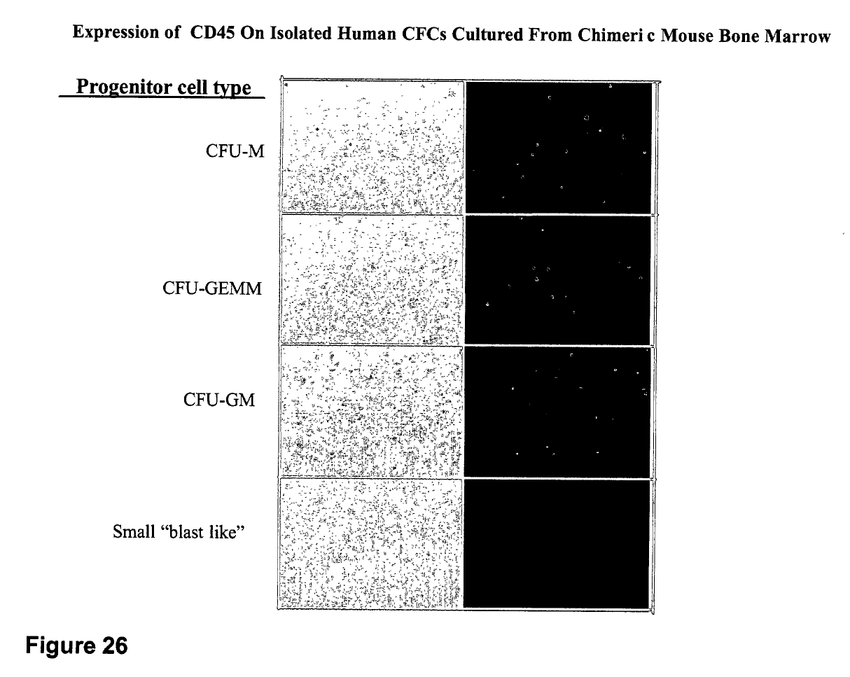

To confirm that colonies grown in the CFC assay (FIG. 25) were derived from human progenitor cells, 40 GEMM colonies, 40 CFU-GM colonies, 40 CFU-M colonies and 5-7 small “CFU-blast like” colonies were plucked from CFC culture dishes, pooled, washed, stained with PE-labeled anti-human CD45 antibody, and then analyzed using fluorescent microscopy. The results illustrated in FIG. 26 demonstrate that all the cells from pooled colonies were derived from human progenitor cells. It should be noted that human erythroid cells do not express CD45. Therefore, it is expected some of the small cells observed within CFU-GEMM colonies should be human CD45 negative.

These results strongly demonstrate that human cord blood SRC can be significantly expanded under ex vivo culture conditions using EDHF as the only source of growth factors. Based on published SRC frequencies, it is estimated that there were approximately 63 SRC cells in the 2.3×10⁶ the total cord blood CD34⁺ cell population prior to EDHF treatment. Moreover, it has been reported that 3-14 SRC are required per graft for successful human progenitor cell engraftment in the presence or absence of exogenous cytokine support. EDHF stimulation ex vivo resulted in a 21.7-fold increase in total cell numbers and as few as 0.2% of these cells (100,000 cells/graft) led to successful engraftment in 80% of the transplanted recipients. Therefore, we know definitively that 100,000 cells contains at least one SRC or as many 3-14 if we rely on the findings from published studies. Since 100,000 cells are only 1/500th of the cells generated we calculate that were between 500 and 7000 SRC generated in the whole population, which computes to an 8-110 fold increase in SRC numbers.

### EXAMPLE 5

### EDHF Promotes Endothelial Cell Growth Under both Serum Rich and Serum-Free Culture Conditions

This example shows culture conditions to support the proliferation of both human and porcine endothelial cells using EDHF as an endothelial cell growth factor supplement. Three primary cell lines were employed: human umbilical vein endothelial cells (HUVEC clone 82901), and two clones of porcine brain endothelial primary cell lines (PMVEC, BPEC-3736 clone 1). The influence of EDHF on endothelial cell proliferation and growth in short-term cultures was evaluated.

A. Endothelial Cells: Human umbilical vein endothelial cells (HUVEC, clone 082901) were harvested from umbilical cord veins by collagenase digestion as previously described (Jaffe E A, Nachman R L, Becker C G, Minick C R: Culture of human endothelial cells derived from umbilical veins, J Clin Invest 52:2745, 1973). A porcine microvascular brain endothelial cell (PBEC-3736, clone-1) culture was isolated from a one-month-old Yucatan Miniature Swine. Isolated endothelial cell clones were cultured on gelatin-coated dishes at 37° C. in a 5% CO₂ incubator and propagated in complete endothelial cell culture medium consisting of M199 medium (GIBCO/Invitrogen, Grand Island, N.Y.) supplemented with 10% heat-inactivated fetal bovine serum (Hyclone, Logan, Utah), 100 μg/mL heparin from pig intestinal mucosa (Sigma Chemical Co, St Louis, Mo.), 100 U/mL penicillin, 100 μg/mL streptomycin, and 15 μg/mL endothelial cell growth supplement prepared from bovine pituitary (Sigma, St Louis, Mo.). Cells were passaged at a 1:4 split ratio from confluent cultures and reached confluency again at about 6 to 7 days. The cells were evaluated for cobblestone morphology and the uptake of acetylated LDL labeled with 1,1′-dioctadecyl-3,3,3′,3′-tetramethylindocarbocyanine perchlorate. EDHF was prepared, screened and tested as previously described in Example 1.

B. Culture of endothelial cells: Culture of purified endothelial cells was performed directly in 96-well flat-bottom bioluminescent plates (Coming; 1 to 2×10³ cells in 0.1 mL/well) at 37° C. in a humidified 5% CO₂ in air atmosphere. Cells were cultured in various base culture mediums supplemented with 10% heat-inactivated fetal bovine serum, 100 μg/mL heparin, 15 μg/mL ECGS and 100 U/mL penicillin and 100 μg/mL streptomycin in the absence or presence of a 100 μg/mL EDHF or specified serial dilutions of EDHF. In addition, the effects of EDHF treatment on the growth response of endothelial cells cultured under serum-free culture conditions were evaluated. Culture mediums tested were M199 (GIBCO/Invitrogen, Grand Island, N.Y.; BioWhittaker, Walkersville, Md.; Cellgro, Herndon, Va.) and Human Endothelial Cell-SF medium (GIBCO/Invitrogen) supplemented with rhFGFβ (20 ng/mL, GIBCO/Invitrogen) and ECGS (15 μg/mL). Cells were cultured for 5-10 days. At the end of the culture period, ATP measurements were made using the LumiTech ViaLight™ bioluminescent method (BioWhitaker, Walkersville, Md.). Cells in each well were lysed using 100 μl of the Nucleotide Releasing Reagent supplied in the Vialight kit, which efficiently releases ATP from cells. Addition of ATP monitoring reagent (luciferin/luciferase reagent) results in release of light, which is proportional to the ATP concentration in the sample. TECAN microplate fluorimeter was used to measure light emission (bioluminescent). Data are expressed as the number of relative light units (RLU) per from the appropriate culture condition.

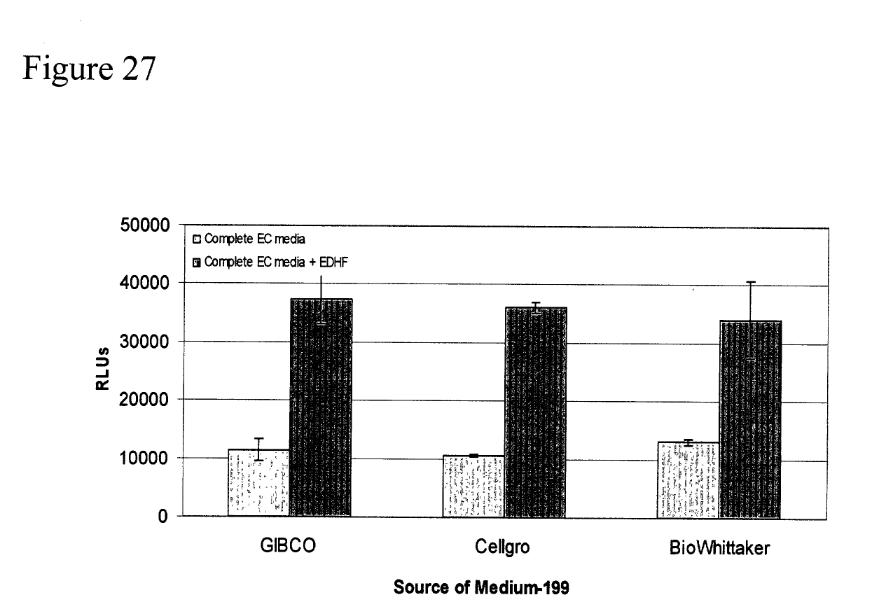

C. EDHF enhances the growth of endothelial cells: As illustrated in FIG. 27, the addition of 100 μg/mL of EDHF to complete serum-containing endothelial cell culture medium significantly increases the proliferative response of PMVECs (porcine microvascular endothelial cells). The day-7 response is identical using various sources of M199, base medium, purchased from the indicated vendors.

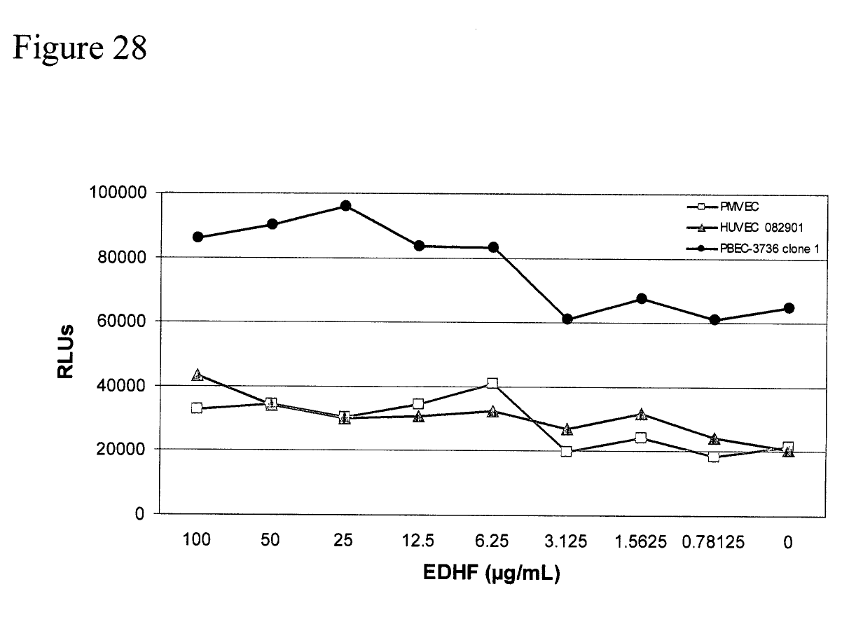

The effects of EDHF, over a range of concentrations (0.78 to 100 μg/mL), on endothelial cell growth were tested using PMVEC, BPEC, and HUVEC primary cell cultures (FIG. 28). In the presence of complete endothelial cell culture medium containing 10% FBS, EDHF at a final plating concentration of 6.25 μg/mL enhances the growth of all three endothelial cell cultures. EDHF amounts greater than 6.25 μg/ml had no additional effect on PMVEC and HUVEC cell growth, whereas the concentration of EDHF to 25 μg/mL supported increased PBEC cell growth.

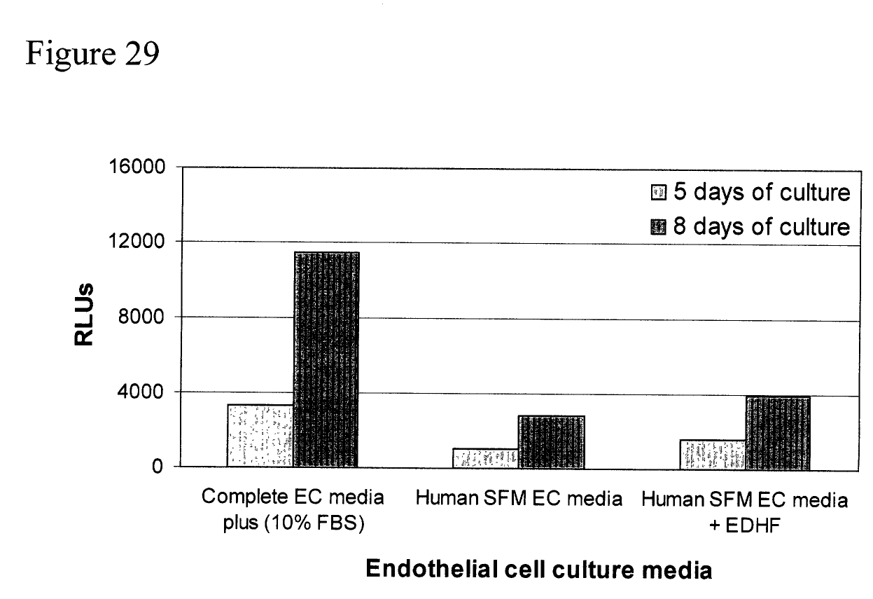

The effects of EDHF on the growth of PMVEC and HUVEC under serum-free culture conditions was evaluated using Human Endothelial Cell-SF medium purchased from GIBCO/Invitrogen and supplemented with 20 ng/mL rhFGF-β and 15 μg/mL (ECGS, Sigma) per manufacture instructions. Results in FIG. 29 demonstrate that 100 μg/mL of EDHF has potent effects on PMVEC using complete endothelial cell growth medium containing 10% FBS but little effect when added to the GIBCO-SFM.

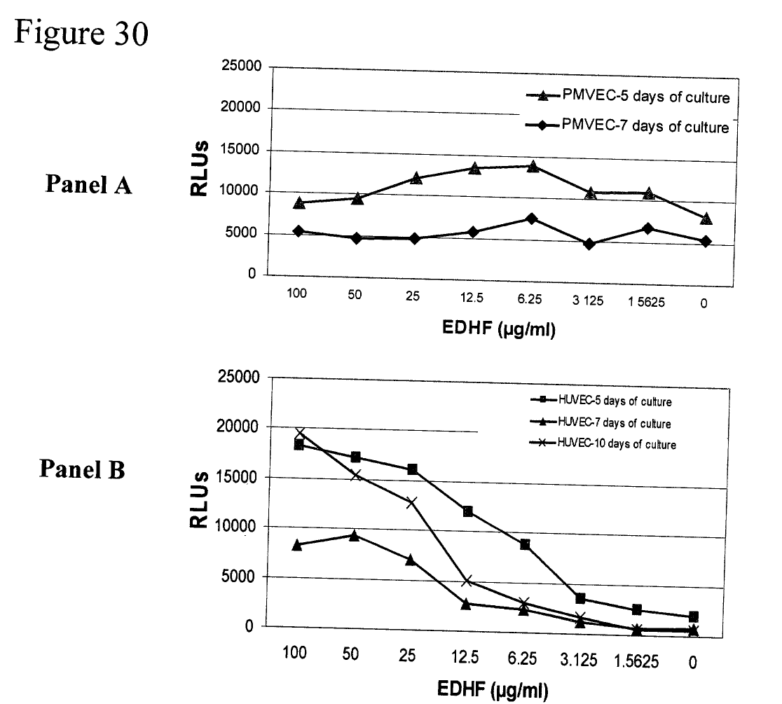

GIBCO-SFM, which is formulated for human endothelial cell culture, may not be the appropriate culture medium for culturing and propagating nonhuman endothelial cells. FIG. 30 shows that over 5-10 days of culture HUVEC grow surprisingly quite poorly in GIBCO-SFM medium containing rhFGFβ and ECGS. However, the addition of EDHF increases cell growth substantially over a wide dose range with 25 μg/mL of EDFH determined to be an optimal plating concentration.

The following is a list of conclusions according to the present invention:

1. A method of enhancing the immune response in a mammal receiving a vaccine, which comprises administering an effective immune enhancing amount of endothelial cell derived hematopoietic growth factor (EDHF) in conjunction with the administration of the vaccine.

2. The method of conclusion 1 wherein the EDHF is administered to the mammal up to 1 to 14 days before administration of the vaccine.

3. The method of conclusion 1 wherein the EDHF is co-administered with the vaccine.

4. The method of conclusion 1 wherein an adjuvant is co-administered with the vaccine.

5. The method of conclusion 4 wherein the adjuvant is a immunostimulatory molecule such as LPS, CD40L and/or CpG DNA.

6. The method of conclusion 1 wherein the mammal is a human.

7. A method of enhancing the immune response in a mammal in need thereof which comprises administering an effective immune enhancing amount of endothelial cell derived hematopoietic growth factor (EDHF) whereby the mammal's dendritic cells and dendritic precursor cells are activated.

8. A method of enhancing the immune response in a mammal in need thereof which comprises administering an effective immune enhancing amount of endothelial cell derived hematopoietic growth factor (EDHF) whereby the mammal's dendritic cells and dendritic precursor cells are elevated in number.

9. A method of enhancing the immune response in a mammal in need thereof which comprises administering an effective immune enhancing amount of endothelial cell derived hematopoietic growth factor (EDHF) whereby the mammal's Langerhans cells are activated.

10. A method of enhancing the immune response in a mammal in need thereof which comprises administering an effective immune enhancing amount of endothelial cell derived hematopoietic growth factor (EDHF) whereby the mammal's Langerhans cells are elevated in number.

11. A method of stimulating hematopoiesis in a mammal, which comprises administering to a mammal an hematopoietic stimulating amount of endothelial cell derived hematopoietic growth factor (EDHF).

12. The method of conclusion 11 wherein the mammal is a human.

13. The method of conclusion 12 wherein the EDHF is administered in an amount of from about 0.01 μg to about 1,000 μg per kg bodyweight.

14. The method of conclusion 11 wherein the EDHF is co-administered with one or more additional hematopoietic growth factors.

15. The method of conclusion 14 wherein the additional hematopoietic growth factors are selected from the group consisting of IL-3, GM-CSF, SCF, EPO, G-CSF, IL-1, IL-6, IL-3, IL-4, TNF-α and FLT3 ligand.

16. The method of conclusion 15 wherein the EDHF is co-administered with TNF-α and FLT3 ligand.

17. In a method of vaccinating a mammal with an antigenic determinant to illicit an immune response by the mammal in order to provide immunity to the mammal from a pathogen possessing the antigenic determinant, the improvement which comprises: administering to the mammal at the time of administration, or from 1-14 days prior to administration, of the vaccination a mixture of endothelial cell derived hematopoietic growth factor (EDHF) proteins having a molecular weight greater than about 30 kDa whereby the mammal has an increased immune response to the antigenic determinant compared to the immune response normally expected from the antigenic determinant alone.

18. A method of improving hematopoietic competence in a mammal comprising:

a) culturing a tissue sample comprising mammalian CD34⁺ hematopoietic cells or analogous non-human hematopoietic cells in a growth medium containing endothelial cell derived hematopoietic growth factor (EDHF) in an amount sufficient to preserve the CD34⁺ hematopoietic cells or analogous non-human hematopoietic cells and to provide cultured cells enriched in the CD34⁺ hematopoietic or analogous non-human hematopoietic cells; and

b) transfusing the enriched cultured cells to the mammal to provide CD34⁺ hematopoietic or analogous non-human hematopoietic cells for generating blood cellular components in the mammal.

19. The method of conclusion 18, wherein the tissue sample is peripheral blood, umbilical cord blood, placental blood, cytokine mobilized peripheral blood or bone marrow.

20. The method of conclusion 18, wherein the tissue sample is autologous or allogeneic to the mammal.

21. The method of conclusion 18, wherein said tissue sample is at least substantially free of stromal cells.

22. The method of conclusion 18, further comprising the step of ablating hematopoietic tissues in the mammal prior to the transfusing step.

23. A method of conducting autologous transplantation in a patient undergoing cytoreductive therapy, which comprises:

(a) administering an effective amount of endothelial cell derived hematopoietic growth factor (EDHF) to the patient to increase the number of circulating hematopoietic stem and progenitor cells available for collection;

(b) collecting hematopoietic stem and progenitor cells from the patient prior to receipt of cytoreductive therapy; and

(c) administering such collected cells to the patient after receipt of cytoreductive therapy.

24. The method of conclusion 23, wherein EDHF is used in combination with a cytokine selected from the group consisting of Flt3 ligand, CSCF-1, GM-CSF, SCF, G-CSF, EPO, IL-1, IL-2, IL-3, IL-4, IL-5, IL-6, IL-7, IL-8, IL-9, IL-10, IL-11, IL-12, IL-13, IL-14, IL-15, GM-CSF/IL-3 fusion proteins, LIF and FGF, and sequential or concurrent combinations thereof

25. The method of conclusion 24, wherein EDHF is used in combination with a cytokine selected from the group consisting of Flt3 ligand, GM-CSF, SCF, G-CSF, EPO, IL-3 and GM-CSF/IL-3 fusion proteins.

26. An improved method for conducting autologous transplantation in a patient receiving cytoreductive therapy, the method comprising:

(a) collecting CD34⁺ hematopoietic cells from the patient prior to receipt of cytoreductive therapy; and

(b) administering such collected cells to the patient after receipt of cytoreductive therapy; wherein the improvement comprises the step of contacting said collected cells ex vivo with an effective amount of endothelial cell derived hematopoietic growth factor (EDHF) prior to administering such collected cells to the patient after receipt of cytoreductive therapy.

27. The method of conclusion 26 wherein EDHF is used in combination with a cytokine selected from the group consisting of Flt3 ligand, CSF-1, GM-CSF, SCF, G-CSF, EPO, IL-1, IL-2, IL-3, IL-4, IL-5, IL-6, IL-7, IL-8, IL-9, IL-10, IL-11, IL-12, IL-13, IL-14, IL-15, GM-CSF/IL-3 fusion proteins, LIF and FGF, and sequential or concurrent combinations thereof.

28. The method of conclusion 27 wherein EDHF is used in combination with a cytokine selected from the group consisting of Flt3 ligand, GM-CSF, SCF, G-CSF, EPO, IL-3 and GM-CSF/IL-3 fusion proteins.

29. An improved method for conducting autologous transplantation in a patient receiving cytoreductive therapy, the method comprising:

(a) collecting CD34⁺ hematopoietic cells from the patient prior to receipt of cytoreductive therapy; and

(b) administering such collected cells to the patient after receipt of cytoreductive therapy; wherein the improvement comprises the step of administering an effective amount of endothelial cell derived hematopoietic growth factor (EDHF) to the patient after receipt of cytoreductive therapy to facilitate the engraftment of the collected cells in the patient.

30. The method of conclusion 29 wherein EDHF is used in combination with a cytokine selected from the group consisting of Flt3 ligand, CSF-1, GM-CSF, SCF, G-CSF, EPO, IL-1, IL-2, IL-3, IL-4, IL-5, IL-6, IL-7, IL-8, IL-9, IL-10, IL-11, IL-12, IL-13, IL-14, IL-15, GM-CSF/IL-3 fusion proteins, LIF and FGF, and sequential or concurrent combinations thereof.

31. A method of treating a mammal undergoing myeloablation therapy, comprising:

(a) obtaining a tissue sample from the mammal, the tissue sample comprising CD34⁺ hematopoietic cells or analogous non-human hematopoietic cells;

(b) culturing the CD34⁺ hematopoietic cells or analogous non-human hematopoietic cells in the presence of an effective amount of endothelial cell derived hematopoietic growth factor (EDHF) to preserve and enrich the hematopoietic cells; and

(c) administering the cultured cells to the mammal following the myeloablation to reconstitute the hematopoietic system of the mammal.

32. The method of conclusion 31, wherein the mammal is a human and the myeloablation therapy is bone marrow irradiation, whole body irradiation, or chemically-induced myeloablation.

33. The method of conclusion 32 wherein the culturing step (b) is done in the presence of one or more additional hematopoietic growth factors in addition to EDHF.

34. The method of conclusion 33 wherein the additional hematopoietic growth factors are selected from the group consisting of IL-3, GM-CSF, SCF, EPO, G-CSF, IL-1, IL-6, IL-3, IL-4, TNF-α and FLT3 ligand.

35. The method of conclusion 34 wherein additional hematopoietic growth factors are IL-3, IL-6, Flt3 ligand or GM-CSF.

36. A method of generating neutrophils in vitro which comprises the sequential steps of:

(a) culturing CD34⁺ hematopoietic cells or analogous non-human hematopoietic cells in the presence of an effective proliferative amount of endothelial cell derived hematopoietic growth factor (EDHF); and

(b) adding to the culture of (a) above after about 14 days of culture an effective amount of at least one or more lineage specific growth factors whereby mature neutrophils are generated.

37. The method of conclusion 36 wherein the lineage specific growth factors are selected from the group consisting of G-CSF, GM-CSF and IL-3.

38. A method of generating dendritic cells in vitro which comprises the sequential steps of:

(a) culturing CD34⁺ hematopoietic cells or analogous non-human hematopoietic cells in the presence of an effective proliferative amount of endothelial cell derived hematopoietic growth factor (EDHF); and

(b) adding to the culture of (a) above after about 14 days of culture an effective amount of at least one or more lineage specific growth factors whereby mature dendritic cells are generated.

39. The method of conclusion 38 wherein the lineage specific growth factors are selected from the group consisting of TNF-α, GM-CSF and IL-4.

40. A method of generating Langerhans cells in vitro which comprises the sequential steps of:

(a) culturing CD34⁺ hematopoietic cells or analogous non-human hematopoietic cells in the presence of an effective proliferative amount of endothelial cell derived hematopoietic growth factor (EDHF); and

(b) adding to the culture of (a) above after about 14 days of culture an effective amount of at least one or more lineage specific growth factors whereby mature Langerhans cells are generated.

41. The method of conclusion 40 wherein the lineage specific growth factors are selected from the group consisting of TNF-α, GM-CSF and IL-4.

42. A method of generating monocytes in vitro which comprises the sequential steps of:

(a) culturing CD34⁺ hematopoietic cells or analogous non-human hematopoietic cells in the presence of an effective proliferative amount of endothelial cell derived hematopoietic growth factor (EDHF); and

(b) adding to the culture of (a) above after about 14 days of culture an effective amount of at least one or more lineage specific growth factors whereby mature monocytes are generated.

43. The method of conclusion 42 wherein the lineage specific growth factors are selected from the group consisting of M-CSF, GM-CSF and IL-3.

44. A method of generating platelets in vitro which comprises the sequential steps of:

(a) culturing CD34⁺ hematopoietic cells or analogous non-human hematopoietic cells in the presence of an effective proliferative amount of endothelial cell derived hematopoietic growth factor (EDHF) for at least 14 days; and

(b) adding to the culture of (a) above after about 14 days of culture an effective amount of at least one or more lineage specific growth factors whereby mature platelets are generated.

45. The method of conclusion 44 wherein the lineage specific growth factors are selected from the group consisting of TPO, SCF and Flt3 ligand.

46. A method of generating erythrocytes in vitro which comprises the sequential steps of:

(a) culturing CD34⁺ hematopoietic cells or analogous non-human hematopoietic cells in the presence of an effective proliferative amount of endothelial cell derived hematopoietic growth factor (EDHF); and

(b) adding to the culture of (a) above after about 14 days an effective amount of at least one or more lineage specific growth factors whereby mature erythrocyte cells are generated.

47. The method of conclusion 46 wherein the lineage specific growth factors are selected from the group consisting of SCF and EPO.

48. A method of generating lymphoid cells in vitro which comprises the sequential steps of:

(a) culturing CD34⁺ hematopoietic cells or analogous non-human hematopoietic cells in the presence of an effective proliferative amount of endothelial cell derived hematopoietic growth factor (EDHF); and

(b) adding to the culture of (a) above after about 14 days of culture an effective amount of at least one or more lineage specific growth factors whereby mature lymphoid cells are generated.

49. The method of conclusion 48 wherein the lineage specific growth factors are selected from the group consisting of SCF, Flt3 ligand, IL-2, IL-4, IL-6, GM-CSF, IL-1b and IL-7.

50. A method of generating stromal cells in vitro which comprises the sequential steps of:

(a) culturing hematopoietic stem and progenitor cells in the presence of an effective proliferative amount of endothelial cell derived hematopoietic growth factor (EDHF) for at least 14 days; and

(b) adding to the culture of (a) above after 14 days of culture an effective amount of at least one or more lineage specific growth factors whereby mature stromal cells are generated.

51. The method of conclusion 50 wherein the EDHF is derived from porcine brain endothelial cells.

52. A method of generating endothelial cells in vitro which comprises the sequential steps of:

(a) culturing hematopoietic stem progenitor cells in the presence of an effective proliferative amount of endothelial cell derived hematopoietic growth factor (EDHF) for at least 14 days; and

(b) adding to the culture of (a) above after 14 days of culture an effective amount of at least one or more lineage specific growth factors whereby mature endothelial cells are generated.

53. The method of conclusion 52 wherein the EDHF is derived from porcine brain endothelial cells.

54. The method of any of conclusions 36-49 further comprising the step of engrafting the hematopoietic cells into a patient in need thereof.

55. The method of conclusion 54 wherein the engrafting step is an autologous engraftment.

56. A method of expanding mammalian pre-dendritic myelomonocytic progenitor cells in vitro which comprises culturing mammalian pre-dendritic myelomonocyctic progenitor cells in the presence of a pre-dendritic myelomonocytic progenitor cell expanding amount of endothelial cell derived hematopoietic growth factor (EDHF).

57. The method of conclusion 56 wherein the pre-dendritic myelomonocytic progenitor cells are human CD34⁺CD38⁺ cells.

58. The method of conclusion 56 wherein the pre-dendritic myelomonocytic progenitor cells are derived from a source selected from the group consisting of bone marrow stem cells, peripheral blood stem cells, cord blood stem cells, fetal liver stem cells and cytokine mobilized stem cells.

59. The method of conclusion 58 wherein the pre-dendritic myelomonocytic progenitor cells are derived from bone marrow.

60. The method of conclusion 56 wherein the pre-dendritic myelomonocytic progenitor cells are cultured in the absence of any other growth factor besides EDHF.

61. The method of conclusion 56 wherein the EDHF is present in the culture medium in an amount of from about 0.1 μg/mL to about 200 μg/mL.

62. The method of conclusion 56 wherein the pre-dendritic myelomonocytic progenitor cells are cultured in the presence of one or more additional hematopoietic growth factors in addition to the EDHF.

63. The method of conclusion 62 wherein the additional hematopoietic growth factors are selected from the group consisting of IL-3, GM-CSF, SCF, EPO, G-CSF, IL-1, IL-6, IL-3, IL-4, TNF-α and FLT3 ligand.

64. A method of expanding CD34⁺ CD38⁻ hematopoietic cells or analogous non-human hematopoietic cells in vitro which comprises culturing CD34⁺CD38⁻ hematopoietic cells or analogous non-human hematopoietic cells in the presence of a CD34⁺CD38⁻ hematopoietic cell expanding amount of endothelial cell derived hematopoietic growth factor (EDHF).

65. The method of conclusion 64 wherein the CD34⁺CD38⁻ hematopoietic cells are human cells.

66. The method of conclusion 64 wherein the CD34⁺CD38⁻ hematopoietic cells are derived from a source selected from the group consisting of bone marrow stem cells, peripheral blood stem cells, cord blood stem cells, fetal liver stem cells and cytokine mobilized stem cells.

67. The method of conclusion 65 wherein the CD34⁺CD38⁻ hematopoietic cells are derived from bone marrow.

68. The method of conclusion 64 wherein the CD34⁺CD38⁻ hematopoietic cells or analogous non-human hematopoietic cells are cultured in the absence of any other growth factor besides EDHF.

69. The method of conclusion 64 wherein the EDHF is present in the culture medium in an amount of from about 0.1 μg/mL to about 200 μg/mL.

70. The method of conclusion 64 wherein the CD34⁺CD38⁻ hematopoietic cells or analogous non-human hematopoietic cells are cultured in the presence of one or more additional hematopoietic growth factors in addition to the EDHF.

71. The method of conclusion 70 wherein the additional hematopoietic growth factors are selected from the group consisting of IL-3, GM-CSF, SCF, EPO, G-CSF, IL-1, IL-6, IL-3, IL-4, TNF-α and FLT3 ligand.

72. The method of any of conclusions 64-71 wherein the analogous non-human hematopoietic cells are selected from the group consisting of primate, murine, porcine, and bovine hematopoietic cells that function in substantially the same manner as CD34⁺CD38⁻ hematopoietic cell

73. The method of conclusion 1 wherein the EDHF is simultaneously administered or sequentially administered with the vaccine and an adjuvant.

74. The method according to any of conclusions 1-73 wherein the EDHF is at least one human endothelial cell protein having a molecular weight greater than about 30 kDa.

75. The method according to any of conclusions 1-73 wherein the EDHF is at least an insulin-like growth factor-binding protein (IGFBP).

76. The method according to any of conclusions 1-73 wherein the EDHF is at least IGFBP-3 having a molecular weight of about 53 kDa.

77. A method of expanding human cord blood-derived CD34⁺ hematopoietic cells ex vivo which comprises culturing human cord blood-derived CD34⁺ hematopoietic cells with an effective CD34⁺ hematopoietic expanding amount of endothelial cell derived hematopoietic growth factor (EDHF).

78. A method of expanding primitive human CD34⁺ hematopoietic stem cells ex vivo which comprises culturing human CD34⁺ hematopoietic stem cells with an effective CD34⁺ hematopoietic stem cell expanding amount of endothelial cell derived hematopoietic growth factor (EDHF).

79. A method of supporting the self renewal division of transplantable hematopoietic cells ex vivo which comprises culturing transplantable hematopoietic cells with an effective self renewal dividing amount of endothelial cell derived hematopoietic growth factor (EDHF).

80. A method of repopulating hematopoietic stem cells in a mammal in need thereof which comprises:

(a) culturing cord blood-derived hematopoietic cells in the presence of an effective hematopoietic cell expanding amount of endothelial cell derived hematopoietic growth factor (EDHF) and

(b) administering the cultured cells to the mammal whereby the administered stem cells repopulate the bone marrow compartment of the mammal.

81. A method of expanding, ex vivo, mammalian hematopoietic cells capable of being engrafted into a mammal which comprises culturing cord blood-derived hematopoietic cells in the presence of a hematopoietic cell expanding amount of endothelial cell derived hematopoietic growth factor (EDHF) whereby the number of hematopoietic cells is increased by at least 10 fold.

82. A method of engrafting human progenitor cells in a patient in need thereof which comprises administering an effective engraftment amount of cord blood-derived hematopoietic cells that were expanded ex vivo by culturing in the presence of endothelial cell derived hematopoietic growth factor (EDHF) whereby lymphoid and myeloid producing cells are engrafted in the patient.

83. A method of expanding eukaryotic cells in vitro which comprises culturing eukaryotic cells in the presence of an effective amount of endothelial cell derived hematopoietic growth factor (EDHF) whereby the eukaryotic cells expand.

84. A method of growing eukaryotic cells in vitro which comprises culturing eukaryotic cells in the presence of an effective growth enhancing amount of endothelial cell derived hematopoietic growth factor (EDHF) whereby the eukaryotic cells grow.

85. A method of maintaining eukaryotic cells in an in vitro culture system which comprises culturing eukaryotic cells in an aqueous culture medium that contains an effective culture maintenance amount of endothelial cell derived hematopoietic growth factor (EDHF).

86. A method of culturing eukaryotic cells in an in vtiro culture system which comprises contacting eukaryotic cells in an aqueous culture medium that contains an effective culture enhancing amount of endothelial cell derived hematopoietic growth factor (EDHF)

87. The method of any of conclusions 83-86 wherein the eukaryotic cells are mammalian, insect, plant or invertebrate cells.

88. The method of any of conclusions 83-86 wherein cells are cultured under static or perfusion ex vivo culture conditions.

89. The method of any of conclusions 83-86 wherein eukaryotic cells are human cells.

90. The method of any of conclusions 83-86 wherein the eukaryotic cells are human cells selected from the group consisting of skin cells, bone cells, cartilage cells, adipocytes, vessel cells, cells of the oral mucous membrane, urothelial cells, endothelial cells, keratinocytes, mesenchymal stem cells, muscle cells, cells of the nervous-system, hematopoietic cells, tendon cells, hair cells, eye cells, germinal cells, cells of the motility system, embryonic cells, stem cells, liver cells, pancreatic cells, kidney cells, heart muscle cells, epithelial cells, mucous membrane cells, hormone-producing cells and transmitter-producing cells.

91. The method of any of conclusions 83-86 wherein cells are genetically modified cells.

92. The method of any of conclusions 83-86 wherein the eukaryotic cells are human cells cultured as an autologous transplant or for the preparation of an autologous, allogeneic or xenogeneic transplant.

93. An in vitro eukaryotic culture composition which comprises:

a. eukaryotic cells,

b. an aqueous culture medium and

c. a growth promoting amount of endothelial cell derived hematopoietic growth factor (EDHF) whereby the eukaryotic cells expand and propagate in the culture composition.

94. In a method of culturing eukaryotic cells in vitro by contacting eukaryotic cells with growth factors in a culture medium under conditions conducive for growth, the improvement which comprises employing endothelial cell derived hematopoietic growth factor (EDHF) or an active fraction thereof as the growth factor.

95. The improved method of conclusion 94 wherein the EDHF or active fraction thereof is the sole growth factor.

96. The improved method of conclusion 94 wherein the EDHF or active fraction thereof is one growth factor in combination with one or more additional growth factors.

97. The improved method of conclusion 94 wherein the eukaryotic cells are human cells selected from the group consisting of skin cells, bone cells, cartilage cells, adipocytes, vessel cells, cells of the oral mucous membrane, urothelial cells, endothelial cells, keratinocytes, mesenchymal stem cells, muscle cells, cells of the nervous-system, hematopoietic cells, tendon cells, hair cells, eye cells, germinal cells, cells of the motility system, embryonic cells, stem cells, liver cells, pancreatic cells, kidney cells, heart muscle cells, epithelial cells, mucous membrane cells, hormone-producing cells and transmitter-producing cells.

98. A method for growing endothelial cells in vitro comprising; culturing endothelial cells in a culture medium supplemented with a growth enhancing amount of endothelial cell derived hematopoietic growth factor (EDHF).

99. An endothelial cell culture comprising a cell culture medium supplemented with a cell proliferative amount of endothelial cell derived hematopoietic growth factor (EDHF) and endothelial cells.

100. A vaccine comprising an effective amount of an antigen and an effective amount of endothelial cell derived hematopoietic growth factor (EDHF).

101. A composition comprising pre-dendritic myelomonocytic progenitor cells wherein the percentage of pre-dendritic myelomonocytic progenitor cells is greater than 85% of all cells present in the composition.

102. The composition of conclusion 101 wherein greater than 95% of all cells are pre-dendritic myelomonocytic progenitor cells.

103. The composition of conclusion 101 wherein greater than 99% of all cells are predendritic myelomonocytic progenitor cells.

104. A composition comprising dendritic cells wherein the percentage of dendritic cells is greater than 85% of all cells present in the composition.

105. The composition of conclusion 104 wherein greater than 95% of all cells are dendritic cells.

106. The composition of conclusion 104 wherein greater than 99% of all cells are dendritic cells.

107. A pharmaceutical composition comprising an effective amount of EDGF and a pharmaceutically acceptable carrier.

108. A genetically modified dendritic cell wherein the dendritic cell contains a vector capable of inducing a gene not naturally expressed by dendritic cells.

109. A method for transforming animal cells comprising culturing the animal cells in the presence of endothelial cell derived hematopoietic growth factor (EDHF) and adding a vector under transforming conditions.

We claim:

1. A method of enhancing the immune response in a mammal receiving a vaccine, which comprises administering an effective immune enhancing amount of endothelial cell derived hematopoietic growth factor (EDHF) in conjunction with the administration of the vaccine.

2. The method of claim 1 wherein the EDHF is administered to the mammal up to 1 to 14 days before administration of the vaccine.

3. The method of claim 1 wherein the EDHF is co-administered with the vaccine.

4. The method of claim 1 wherein an adjuvant is co-administered with the vaccine.

5. The method of claim 4 wherein the adjuvant is a immunostimulatory molecule such as LPS, CD40L and/or CpG DNA.

6. The method of claim 1 wherein the mammal is a human.

7. A method of stimulating hematopoiesis in a mammal, which comprises administering to a mammal an hematopoietic stimulating amount of endothelial cell derived hematopoietic growth factor (EDHF).

8. The method of claim 7 wherein the mammal is a human.

9. The method of claim 8 wherein the EDHF is administered in an amount of from about 0.01 μg to about 1,000 μg per kg bodyweight.

10. The method of claim 7 wherein the EDHF is co-administered with one or more additional hematopoietic growth factors.

11. The method of claim 10 wherein the additional hematopoietic growth factors are selected from the group consisting of IL-3, GM-CSF, SCF, EPO, G-CSF, IL-1, IL-6, IL-3, IL-4, TNF-α and FLT3 ligand.

12. The method of claim 11 wherein the EDHF is co-administered with TNF-α and FLT3 ligand.

13. A method of expanding mammalian pre-dendritic myelomonocytic progenitor cells in vitro which comprises culturing mammalian pre-dendritic myelomonocyctic progenitor cells in the presence of a pre-dendritic myelomonocytic progenitor cell expanding amount of endothelial cell derived hematopoietic growth factor (EDHF).

14. The method of claim 13 wherein the pre-dendritic myelomonocytic progenitor cells are human CD34⁺ CD38⁺ cells.

15. The method of claim 13 wherein the pre-dendritic myelomonocytic progenitor cells are derived from a source selected from the group consisting of bone marrow stem cells, peripheral blood stem cells, cord blood stem cells, fetal liver stem cells and cytokine mobilized stem cells.

16. The method of claim 15 wherein the pre-dendritic myelomonocytic progenitor cells are derived from bone marrow.

17. The method of claim 13 wherein the pre-dendritic myelomonocytic progenitor cells are cultured in the absence of any other growth factor besides EDHF.

18. The method of claim 13 wherein the EDHF is present in the culture medium in an amount of from about 0.1 μg/mL to about 200 μg/mL.

19. The method of claim 13 wherein the pre-dendritic myelomonocytic progenitor cells are cultured in the presence of one or more additional hematopoietic growth factors in addition to the EDHF.

20. The method of claim 19 wherein the additional hematopoietic growth factors are selected from the group consisting of IL-3, GM-CSF, SCF, EPO, G-CSF, IL-1, IL-6, IL-3, IL-4, TNF-α and FLT3 ligand.
